(19) United States
(12) Patent Application Publication
(10) Pub. No.: US 2023/0172715 A1
(43) Pub. Date: Jun. 8, 2023

(54) EXPANDABLE SHEATH FOR INTRODUCING AN ENDOVASCULAR DELIVERY DEVICE INTO A BODY

(71) Applicant: Edwards Lifesciences Corporation, Irvine, CA (US)

(72) Inventors: Jeong Soo Lee, Diamond Bar, CA (US); Leah Paige Gaffney, Orange, CA (US); Judy Kim, Anaheim, CA (US); David Mora, Lake Forest, CA (US); Elizabeth Anne Anderson, Costa Mesa, CA (US); Sonny Tran, Westminster, CA (US); Nasser William Saleh, Stockton, CA (US); Ilan Tamir, Irvine, CA (US); Maxwell Harrison Fine, Costa Mesa, CA (US); Duy Nguyen, Corona, CA (US); Kim D. Nguyen, Irvine, CA (US); Thanh V. Nguyen, Irvine, CA (US); Erik Bulman, Lake Forest, CA (US)

(21) Appl. No.: 18/103,361

(22) Filed: Jan. 30, 2023

Related U.S. Application Data
(63) Continuation of application No. PCT/US2021/031275, filed on May 7, 2021.
(60) Provisional application No. 63/059,772, filed on Jul. 31, 2020.

(51) Int. Cl. A61F 2/24; A61M 25/00; A61L 29/12; A61L 29/14

(52) U.S. Cl. CPC A61F 2/2436; A61M 25/0009; A61L 29/126; A61L 29/14; A61L 2400/10; B29C 48/09

(57) ABSTRACT

Aspects of an expandable sheath can be used in conjunction with a catheter assembly to introduce a prosthetic device, such as a heart valve, into a patient. Such aspects can minimize trauma to the vessel by allowing for temporary expansion of a portion of the introducer sheath to accommodate the delivery apparatus, followed by a return to the original diameter once the prosthetic device passes through. Some aspects can include various configurations of the sheath that comprise an elongated tube having a disclosed composition that can form an outer jacket or a strain relief jacket or can be used as the outer layer of the sheath. Aspects of the present expandable sheath can avoid the need for multiple insertions for the dilation of the vessel and reduce the push force needed for passage of the medical device, thus offering advantages over prior art introducer sheaths.

### CROSS-REFERENCE TO RELATED APPLICATIONS

This application is a continuation of International Application No. PCT/US2021/031275, filed May 7, 2021, which claims the benefit of U.S. Provisional Application No. 63/059,772, filed Jul. 31, 2020, the content of which is incorporated herein by reference in its entirety.

### FIELD

The present application concerns aspects of a sheath for use with catheter-based technologies for repairing and/or replacing heart valves, as well as for delivering a prosthetic device, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

An introducer sheath can be used to safely introduce a delivery apparatus into a patient's vasculature (e.g., the femoral artery). An introducer sheath generally has an elongated sleeve that is inserted into the vasculature and a housing that contains one or more sealing valves that allow a delivery apparatus to be placed in fluid communication with the vasculature with minimal blood loss. A conventional introducer sheath typically requires a tubular loader to be inserted through the seals in the housing to provide an unobstructed path through the housing for a valve mounted on a balloon catheter. A conventional loader extends from the proximal end of the introducer sheath and therefore decreases the available working length of the delivery apparatus that can be inserted through the sheath and into the body.

Conventional methods of accessing a vessel, such as a femoral artery, prior to introducing the delivery system include dilating the vessel using multiple dilators or sheaths that progressively increase in diameter. This repeated insertion and vessel dilation can increase the amount of time the procedure takes, as well as the risk of damage to the vessel.

Radially expanding intravascular sheaths have been disclosed. Such sheaths tend to have complex mechanisms, such as ratcheting mechanisms that maintain the shaft or sheath in an expanded configuration once a device with a larger diameter than the sheath's original diameter is introduced.

However, delivery and/or removal of prosthetic devices and other materials to or from a patient still poses a significant risk to the patient. Furthermore, accessing the vessel remains a challenge due to the relatively large profile of the delivery system that can cause longitudinal and radial tearing of the vessel during insertion. The delivery system can additionally dislodge calcified plaque within the vessels, posing an additional risk of clots caused by the dislodged plaque.

Accordingly, there remains a need in the art for an improved introducer sheath for endovascular systems used for implanting valves and other prosthetic devices.

### SUMMARY

The disclosed herein, an expandable sheath can minimize trauma to the vessel by allowing for temporary expansion of a portion of the introducer sheath to accommodate a delivery system, followed by a return to the original diameter once the delivery system passes through. Some aspects can comprise a sheath with a smaller profile than that of prior art introducer sheaths. Furthermore, as described in certain aspects, the disclosed sheath can reduce the length of time a procedure takes, as well as reduce the risk of a longitudinal or radial vessel tear or plaque dislodgement because only one sheath is required, rather than several different sizes of sheaths. In still further aspects, the present expandable sheath can require only a single vessel insertion, as opposed to requiring multiple insertions for the dilation of the vessel.

In one aspect disclosed herein is a sheath for delivering a medical device, wherein the sheath has a proximal and a distal end and comprises an elongated tube forming an outer layer of the sheath that is positioned at at least the proximal end of the sheath and extending along at least a portion of a length of the sheath, having an inner surface and an outer surface, and wherein the elongated tube comprises a first polymer layer, wherein the first polymer layer comprises a first compound composition comprising: from greater than 0 wt % to less than 100 wt % of a first polymer comprising a polyether block amide, a polyurethane, or a combination thereof; less than about 65% of an inorganic filler based on a total weight of the first compound composition; and up to about 20% of a solid lubricant filler based on a total weight of the first compound composition; wherein the elongated tube is configured to reversibly expand from an initial diameter do in an unexpended position to an expanded diameter dₑ in an expanded position upon passage of a medical device; and wherein the sheath exhibits at least a 10% reduction in an insertion force when compared with a substantially identical reference sheath that does not comprise the first polymer layer.

In one aspect, the first polymer can have a substantially same durometer along a total length of the elongated tube. Yet, in other aspects, a durometer of the first polymer at a proximal end of the elongated tube can be different from a durometer of the first polymer at a distal end of the elongated tube.

In one aspect, wherein the elongated tube comprises two or more polymer layers. In such an exemplary aspect, the elongated tube comprises at least a second polymer layer comprising a second compound composition comprising from greater than 0 wt % to 100 wt % of a second polymer comprising polyether block amide, a polyurethane, or a composition thereof. In certain exemplary aspects, the second compound composition can further comprise up to 20% of tackiness reducing additive based on a total weight of the second compound composition.

In still further aspects, the second polymer layer can comprise PEBAX®. While in other aspects, the second polymer layer can comprise polyurethane.

In some aspects, the sheath disclosed herein can exhibit at least a 20% reduction in an insertion force when compared with a substantially identical reference sheath that does not comprise the first polymer layer.

In still further aspects, the disclosed sheath for introducing a prosthetic device comprises an inner layer and an outer layer. In one aspect, the sheath as disclosed herein further comprises an expandable tubular inner liner extending along the length of the sheath and comprising at least one folded portion, wherein the expandable inner liner has an inner surface and an outer surface, wherein the inner surface of the expandable inner liner defines a lumen and forms an inner surface of the at least one folded portion, and wherein the outer surface extends circumferentially to form an outer surface of the at least one folded portion; and a first outer tubular layer extending at least partially along the length of the sheath and having an inner surface and an outer surface, wherein the inner surface of the first outer tubular layer further extends at least partially around the outer surface of the inner liner such that at least a portion of the inner surface of the first outer tubular layer is positioned adjacent to the outer surface of the at least one folded portion of the inner liner; wherein the elongated tube is positioned such that at least a portion of the inner surface of the elongated tube overlies at least a portion of the outer surface of the first outer tubular layer.

In still further aspects, the disclosed herein sheath has a proximal and a distal end and comprises: an expandable tubular inner liner comprising at least one folded portion, wherein the expandable inner liner has an inner surface and an outer surface, wherein the inner surface of the expandable inner liner defines a lumen and forms an inner surface of the at least one folded portion, and wherein the outer surface extends circumferentially to form an outer surface of the at least one folded portion, a first outer tubular layer having an inner surface and an outer surface, wherein the inner surface of the first outer tubular layer extends at least partially around the outer surface of the inner liner such that at least a portion of the inner surface of the first outer tubular layer is positioned adjacent to the outer surface of the at least one folded portion of the inner liner; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0 wt % to less than 100 wt % of a polymer comprising a polyether block amide, a polyurethane, or a combination thereof; less than about 65% of an inorganic filler based on a total weight of the first compound composition; and up to about 20% of a solid lubricant filler based on a total weight of the first compound composition.

Also disclosed herein is an aspect describing a sheath for delivering a medical device, wherein the sheath has a proximal and a distal end and comprises an expandable tubular inner liner comprising at least one folded portion, wherein the expandable inner liner has an inner surface and an outer surface, wherein the inner surface of the expandable inner liner defines a lumen and forms an inner surface of the at least one folded portion, and wherein the outer surface extends circumferentially to form an outer surface of the at least one folded portion and wherein the outer surface of the inner liner is selectively etched; a first outer tubular layer having an inner surface and an outer surface, wherein the inner surface of the outer layer extends at least partially around the outer surface of the inner liner such that at least a portion of the inner surface of the outer layer is positioned adjacent to at least a portion of the outer surface of the at least one folded portion of the inner liner; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof; less than about 65% of an inorganic filler based on a total weight of the first compound composition; and up to about 20% of a solid lubricant filler based on a total weight of the first compound composition.

Also disclosed herein are aspects, where in an addition to the elongated tube as described in any of the preceding aspects, the sheath further comprises a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of a cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and wherein the elongated tube is positioned such that the inner surface of the elongated tube overlies at least a portion of the outer surface of the inner liner.

Also disclosed herein is a sheath for delivering a medical device, wherein the sheath has a proximal and a distal end and comprises: a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and an elongated tube forming an outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the inner liner, wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof; less than about 65% of an inorganic filler based on a total weight of the first compound composition; and up to about 20% of a solid lubricant filler based on a total weight of the first compound composition.

Also disclosed herein is an expandable sheath comprising: an inner tubular layer comprising a longitudinal slit and partially defining an inner lumen; a first outer tubular layer enveloping the inner layer, the outer tubular layer comprising a longitudinally extending, folded flap that overlies a portion of an outer surface of the outer layer when the sheath is in an unexpanded state; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof; less than about 65% of an inorganic filler based on a total weight of the first compound composition; and up to about 20% of a solid lubricant filler based on a total weight of the first compound composition; wherein an outwardly directed radial force from a prosthetic device moving through the inner lumen widens the longitudinal slit and unfolds the folded flap to allow expansion of the sheath.

Still further disclosed herein, is a sheath for delivering a medical device, the sheath comprising: a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof; less than about 65% of an inorganic filler based on a total weight of the first compound composition; and up to about 20% of a solid lubricant filler based on a total weight of the first compound composition; wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

Also disclosed herein is a sheath delivering a medical device comprising: a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; a coiled wire along a length of the sheath, the coil wire providing uniform bending of the sheath to prevent kinking; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof; less than about 65% of an inorganic filler based on a total weight of the first compound composition; and up to about 20% of a solid lubricant filler based on a total weight of the first compound composition, wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

Also disclosed herein is a method of making a sheath having a proximal end and a distal end and comprising: a) extruding a tubular body to form an elongated tube comprising a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; b) disposing the elongated tube on the sheath such that the elongated tube forms an outer layer of the sheath, and wherein the elongated tube is positioned at at least the proximal end of the sheath and extending along at least a portion of a length of the sheath, wherein the elongated tube is configured to reversibly expand from an initial diameter do in an unexpended position to an expanded diameter dₑ in an expanded position upon passage of a medical device; and wherein the formed sheath exhibits at least a 10% reduction in an insertion force when compared with a substantially identical reference sheath that does not comprise the first polymer layer.

Also disclosed herein is the method further comprising providing: a) a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; b) a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; and c) a coiled wire along a length of the sheath, the coil wire providing uniform bending of the sheath to prevent kinking; and disposing the elongated tube such that the inner surface of the elongated tube overlies at least a portion of the outer surface of the first outer tubular layer, and wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

Also further disclosed herein is the method further comprising: providing: a) a circumferentially continuous first elastic outer tubular layer defining an initial elastic lumen extending axially therethrough, the initial elastic lumen having an initial diameter; and b) an inner tubular layer extending through the initial elastic lumen of the first elastic outer tubular layer and comprising at least three circumferentially spaced, longitudinally extending thick wall segments and at least three circumferentially spaced, longitudinally extending thin wall segments, each thin wall segment extending between two adjacent thick wall segments to define an expanded lumen extending axially through the inner tubular layer, the expanded lumen having an expanded diameter larger than the initial diameter of the initial elastic lumen; c) wherein the inner tubular layer, in a compressed condition, forms at least three circumferentially spaced folds, each of the circumferentially spaced folds including a three-layer thickness in a radial direction comprised of portions of two adjacent thick wall segments and a thin wall segment sandwiched therebetween; wherein the inner tubular layer, in a locally expanded condition, has the thick wall segments and the thin wall segments unfolded and expanded apart; and wherein the inner tubular layer is configured to be urged by the first elastic outer tubular layer at least partially back to the compressed condition after passage of an implant through the expanded lumen; and disposing the elongated tube such that the inner surface of the elongated tube overlies at least a portion of the outer surface of the first elastic outer layer.

Also disclosed herein is a method comprising: providing: a) an expandable tubular inner liner extending along the length of the sheath and comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion; b) a first outer tubular layer extending at least partially along the length of the sheath and having an inner surface and an outer surface, wherein the inner surface of the first outer tubular layer further extends at least partially around the outer surface of the inner liner such that at least a portion of the inner surface of the first outer tubular layer is positioned adjacent to the outer surface of the at least one folded portion of the inner liner; and disposing the elongated tube such that the inner surface of the elongated tube overlies at least a portion of the outer surface of the first outer tubular layer, and wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

The foregoing and other features and advantages of the disclosure will become more apparent from the following detailed description, which proceeds with reference to the accompanying figures.

### BRIEF DESCRIPTION OF THE DRAWINGS

FIG. 1 is an elevation view of a sheath according to the present disclosure, along with an endovascular delivery apparatus for implanting a prosthetic valve.

FIGS. 2A, B, and D are section views of an exemplary sheath for introducing a prosthetic device into a patient, and FIG. 2C is a perspective view of one component of such a sheath.

FIG. 3 is an elevation view of the sheath shown in FIG. 2.

FIGS. 4A-4B show elevation views of two aspects of a sheath according to the present disclosure, having varying outer diameters.

FIG. 5 illustrates an elevation view of one aspect of a sheath, expanded at a first location to accommodate a delivery system.

FIG. 6 shows an elevation view of the sheath, expanded at a second location farther down the sheath.

FIG. 7 shows a section view of another aspect of a sheath that further comprises an outer covering or shell.

FIG. 8 illustrates an elevation view of one aspect of a sheath with an outer covering or shell.

FIG. 9 illustrates a partial elevation view of one aspect of an intermediate tubular layer that can be used to construct a sheath according to the present disclosure.

FIG. 10 illustrates a partial elevation view of another aspect of an intermediate tubular layer having a variable diamond design.

FIG. 11 illustrates a partial elevation view of another aspect of an intermediate tubular layer having a diamond design with spring struts.

FIG. 12 illustrates a partial elevation view of another aspect of an intermediate tubular layer having a diamond design with straight struts.

FIG. 13 illustrates a partial elevation view of another aspect of an intermediate tubular layer having a saw tooth design with spring struts.

FIG. 14 illustrates a partial elevation view of another aspect of an intermediate tubular layer having a saw tooth design with straight struts.

FIG. 15 illustrates a partial elevation view of another aspect of an intermediate tubular layer having a diamond design with straight struts.

FIG. 16 illustrates a partial elevation view of another aspect of an intermediate tubular layer having a helical or spiral design.

FIG. 17 illustrates a partial elevation view of another aspect of an intermediate tubular layer having a diamond design with non-straight struts.

FIG. 18 illustrates a partial elevation view of another aspect of an intermediate tubular layer having an alternative diamond design with non-straight struts.

FIG. 19 illustrates a partial elevation view of another aspect of an intermediate tubular layer having yet another diamond design with non-straight struts.

FIG. 20 illustrates a partial elevation view of another aspect of an intermediate tubular layer having a diamond design with struts.

FIG. 21 illustrates a partial elevation view of another aspect of an intermediate tubular layer having a design similar to that shown in FIG. 20, but with additional struts.

FIG. 22 illustrates a partial elevation view of another aspect of an intermediate tubular layer having a diamond design with spiral struts.

FIG. 23 illustrates a partial elevation view of another aspect of an intermediate tubular layer having a diamond design with adjacent struts.

FIG. 24 illustrates a section view of one aspect of a sheath having a longitudinal notch.

FIG. 25 shows a section view of one aspect of a sheath having a longitudinal cut in the inner layer.

FIG. 26 shows a perspective view of an exemplary sheath having a plurality of notches or cuts in the outer tubular layer in one aspect.

FIG. 27A illustrates a section view of one aspect of a sheath, wherein the outer tubular layer contains a longitudinal cut, and the inner layer extends into the gap created by the cut in the outer tubular layer, in an unexpanded configuration; and FIGS. 27B-27E show section views of various aspects of a sheath in the unexpanded configuration.

FIG. 28 shows a section view of the sheath of FIG. 27A in an expanded configuration.

FIGS. 29A-29D show section views of various aspects of a sheath having overlapping sections.

FIG. 30 illustrates a block diagram of one aspect of a method of making a sheath according to the present disclosure.

FIG. 31 illustrates a block diagram of another aspect of a method of making a sheath according to the present disclosure.

FIGS. 32A-32H illustrates section or elevation views of various method steps of the methods shown in FIGS. 30-31.

FIG. 33 illustrates a plan view of one aspect of a sheath having a partial slit or score line.

FIG. 34 illustrates a plan view of another aspect of a sheath having a partial slit or score line.

FIG. 35 is an elevation view of an expandable sheath according to the present disclosure and a representative housing.

FIG. 36 is an enlarged cutaway view of the distal end of the sheath of FIG. 35.

FIG. 37 is a section view of the distal end of the sheath of FIG. 35, taken along line 37-37 in FIG. 36.

FIG. 38 is a section view of a proximal section of the sheath of FIG. 35, taken along line 38-38 in FIG. 35.

FIG. 39 is a section view of the sheath of FIG. 35 in a rest (unexpanded) configuration, taken along line 39-39 in FIG. 35.

FIG. 40 is the section view of the sheath of FIG. 39, in an expanded configuration.

FIG. 41 shows an elevation view of an expandable sheath having an elastic outer cover, according to another aspect.

FIG. 42 illustrates a section view of the sheath of FIG. 41, taken along line 42-42 in FIG. 41.

FIG. 43 illustrates the sectional view of the sheath shown in FIG. 42, in an expanded configuration.

FIG. 44 illustrates a section view of another aspect of an expandable sheath.

FIG. 45 shows an expanded configuration of the sheath of FIG. 44.

FIG. 46 illustrates a section view of another aspect of an expandable sheath.

FIG. 47 shows an expanded configuration of the sheath of FIG. 46.

FIG. 48 illustrates a section view of another aspect of an expandable sheath according to the present disclosure.

FIG. 49 illustrates a section view of another aspect of an expandable sheath.

FIG. 50 is a section view of an example sheath in an unexpanded configuration.

FIG. 51 is a section view of the sheath of FIG. 50 in an expanded configuration.

FIG. 52 is a section view of the sheath of FIG. 50, including an outer jacket.

FIG. 53 is a section view of the sheath of FIG. 35 in a rest (unexpanded) configuration, including an outer jacket, taken along line 39-39 in FIG. 35.

FIG. 54 is a section view of the sheath of FIG. 53 in a rest (unexpanded) configuration, taken along line 39-39 in FIG. 35.

FIG. 55 is a section view of the sheath of FIG. 54, in an expanded configuration.

FIG. 56 is a section view of the sheath of FIG. 54 in a rest (unexpanded) configuration, including a lubricant between the outer layer and the outer jacket.

FIG. 57 is a section view of the sheath of FIG. 54 in a rest (unexpanded) configuration, including a lubricant and a bonding strip.

FIG. 58 is a bottom perspective view of the sheath of FIG. 57.

FIG. 59 is a bottom perspective view of the sheath of FIG. 57.

FIG. 60A is a top perspective view of the sheath of FIG. 54.

FIG. 60B is a section view of an exemplary sheath in one aspect.

FIGS. 61A-61B are section views of aspects of a sheath for introducing a medical device into a patient, and FIG. 61C is a perspective view of one of the components of the exemplary sheath.

FIGS. 62A-B illustrate a section view of one aspect of an exemplary inner liner: FIG. 62A depicts an unexpanded configuration, while FIG. 62B depicts an expanded configuration.

FIGS. 63A-63E and 63H show section views of various aspects of exemplary sheaths. FIGS. 63F, 63G, and 63I show perspective views of various aspects of exemplary sheaths.

FIGS. 64A-64D are section views of the distal end of the exemplary sheath; FIG. 64A shows a section view of the exemplary sheath with a lubricant disposed between the sliding and overlaying portions of the sheet and the braid that is not embedded into the elastomeric polymer layer; FIG. 64B shows a section view of the exemplary sheath with a lubricant disposed between the sliding and overlaying portions of the sheet and a lubricant disposed between the inner liner and outer layer, where the braid that is not embedded into the elastomeric polymer layer; FIG. 64C shows a section view of the exemplary sheath with a lubricant disposed between the inner liner and outer layer, where the braid that is not embedded into the elastomeric polymer layer, with and without the lubricant; FIG. 64D shows a section view of the exemplary sheath with a lubricant disposed between the sliding and overlaying portions of the sheet and a lubricant disposed between the inner liner and outer layer, where the braid is at least partially embedded into the elastomeric polymer layer.

FIGS. 65A-D illustrate a section view of a proximal section of the sheath; FIG. 65A shows a section view of the exemplary sheath with a lubricant disposed between the sliding and overlaying portions of the sheet and the braid that is not embedded into the elastomeric polymer layer; FIG. 65B shows a section view of the exemplary sheath with a lubricant disposed between the sliding and overlaying portions of the sheet and a lubricant disposed between the inner liner and outer layer, where the braid that is not embedded into the elastomeric polymer layer; FIG. 65C shows a section view of the exemplary sheath with a lubricant disposed between the inner liner and outer layer, where the braid that is not embedded into the elastomeric polymer layer, with and without the lubricant; FIG. 65D shows a section view of the exemplary sheath with a lubricant disposed between the sliding and overlaying portions of the sheet and a lubricant disposed between the inner liner and outer layer, where the braid is at least partially embedded into the elastomeric polymer layer.

FIG. 66 is a section view of the sheath in a rest (unexpanded) configuration, taken along the distal end.

FIG. 67 shows a section view of the sheath of FIG. 66 in an expanded configuration.

FIG. 68 illustrates a block diagram of one aspect of a method of making a sheath according to the present disclosure.

FIG. 69 illustrates a block diagram of another aspect of a method of making a sheath according to the present disclosure.

FIGS. 70A-70J illustrates section or side views of various method steps of the methods shown in FIGS. 68-69.

FIG. 71 is a section view of an exemplary sheath in one aspect.

FIG. 72 is a section view of an exemplary sheath in one aspect.

FIG. 73 is an elevation view of an exemplary elongated tube according to another aspect.

FIG. 74 is a cross-section view of an exemplary elongated tube taken along section line A-A of FIG. 73.

FIG. 75 is a section view of an exemplary elongated tube in a rest (unexpanded) configuration, taken along section lines B-B of FIG. 73.

FIG. 76 is a partial section view of an exemplary elongated tube in one aspect.

FIG. 77 is a section view of another exemplary elongated tube in a rest (unexpanded) configuration, including a single reinforcing member, taken along section lines B-B of FIG. 73.

### DETAILED DESCRIPTION

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

### Definitions

As used in this application and in the claims, the singular forms “a,” “an,” and “the” include the plural forms unless the context clearly dictates otherwise. Thus, for example, reference to a “polymer” includes aspects having two or more such polymers unless the context clearly indicates otherwise.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable combination.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and the claims, the term “comprising” can include the aspects “consisting of” and “consisting essentially of.” Additionally, the term “includes” means “comprises.”

For the terms “for example” and “such as,” and grammatical equivalences thereof, the phrase “and without limitation” is understood to follow unless explicitly stated otherwise.

References in the specification and concluding claims to parts by weight of a particular element or component in a composition or article denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5 and are present in such ratio regardless of whether additional components are contained in the composition.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

As used herein, the term “composition” is intended to encompass a product comprising the specified ingredients in the specified amounts, as well as any product which results, directly or indirectly, from a combination of the specified ingredients in the specified amounts.

A weight percent of a component, unless specifically stated to the contrary, is based on the total weight of the formulation or composition in which the component is included.

As used herein, the term “substantially” means that the subsequently described event or circumstance completely occurs or that the subsequently described event or circumstance generally, typically, or approximately occurs.

As used herein, the term “substantially,” when used in reference to a composition, refers to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature or component.

As used herein, the term “substantially,” in, for example, the context “substantially free” refers to a composition having less than about 1% by weight, e.g., less than about 0.5% by weight, less than about 0.1% by weight, less than about 0.05% by weight, or less than about 0.01% by weight of the stated material, based on the total weight of the composition.

As used herein, the terms “substantially identical reference composition” or “substantially identical reference article” refer to a reference composition or article comprising substantially identical components in the absence of an inventive component. In another exemplary aspect, the term “substantially,” in, for example, the context “substantially identical reference composition,” refers to a reference composition comprising substantially identical components and wherein an inventive component is substituted with a common in the art component.

Further, the terms “coupled” and “associated” generally mean electrically, electromagnetically, and/or physically (e.g., mechanically or chemically) coupled or linked and do not exclude the presence of intermediate elements between the coupled or associated items.

It will be understood that, although the terms “first,” “second,” etc., may be used herein to describe various elements, components, regions, layers and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example aspects.

It is understood that the terms “layer” and “liner” can be used interchangeably. It is further understood that for the purposes of the current disclosure, the term “outer jacket” refers to the elongated tube having the disclosed herein composition and characteristics.

It is further understood that the phrases “insertion force” and “push force” can be used interchangeably.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term “atraumatic” is commonly known in the art and refers to a device or a procedure that minimizes tissue injury.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art using only routine experimentation.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may, in some cases, be rearranged or performed concurrently. Further, descriptions and disclosures provided in association with one particular aspect are not limited to that aspect and may be applied to any aspect disclosed.

While aspects of the present disclosure can be described and claimed in a particular statutory class, such as the system statutory class, this is for convenience only, and one of ordinary skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The present disclosure may be understood more readily by reference to the following detailed description of various aspects of the disclosure and the examples included therein and to the Figures and their previous and following description.

The present disclosure may be understood more readily by reference to the following detailed description of various aspects of the disclosure and the examples included therein and to the Figures and their previous and following description.

### Sheath

Disclosed aspects of an expandable sheath can minimize trauma to the vessel by allowing for temporary expansion of a portion of the introducer sheath to accommodate the delivery system, followed by a return to the original diameter once the device passes through. In some aspects, the sheath can comprise a sheath with a smaller profile (e.g., a smaller diameter in the rest configuration) than that of prior art introducer sheaths. Furthermore, as disclosed in the present aspects, the sheath can reduce the length of time a procedure takes, as well as reduce the risk of a longitudinal or radial vessel tear or plaque dislodgement because only one sheath is required, rather than several different sizes of sheaths. In certain aspects, the present expandable sheath can avoid the need for multiple insertions for the dilation of the vessel. Such expandable sheaths can be useful for many types of minimally invasive surgery, such as any surgery requiring introduction of an apparatus into a subject's vessel. For example, the sheath can be used to introduce other types of delivery apparatus for placing various types of intraluminal devices (e.g., stents, prosthetic heart valves, stented grafts, etc.) into many types of vascular and non-vascular body lumens (e.g., veins, arteries, esophagus, ducts of the biliary tree, intestine, urethra, fallopian tube, other endocrine or exocrine ducts, etc.).

Also, disclosed herein aspects refer to the sheaths having a reduced insertion force as compared to any other available commercial sheaths. As one of ordinary skill in the art would readily appreciate, the reduction in the insertion force of the sheath and any medical device passing through results in reduced or substantially eliminated danger to the patients during the medical procedure. It is understood that the insertion force of the inventive sheaths, reference sheaths, and any other commercially available sheaths is measured using the same standardized technique for a proper comparison. In such aspects, a tensile tester, Instron 3366, has been used to measure a simulated insertion force that is required for a prosthetic valve to enter the described sheath. An additional constriction tube has been used to simulate vascular elasticity that contributes to push force. In certain exemplary aspects, the test is performed in a water bath in a temperature range from room temperature to a normal body temperature (for example and without limitation from about 20° C. to less than about 40° C.). The specimen can be kept in a straight configuration. The test can be performed with a tapered mandrel to simulate a valve entering the sheath.

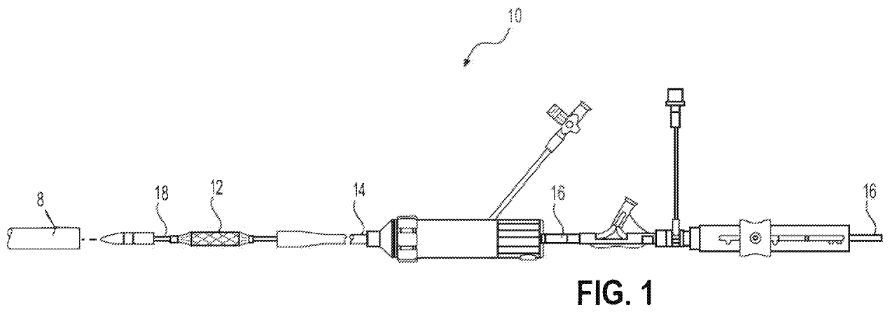

FIG. 1 illustrates a sheath 8 according to the present disclosure, in use with a representative delivery apparatus 10, for delivering a prosthetic device 12, such as a tissue heart valve to a patient. The apparatus 10 can include a steerable guide catheter 14 (also referred to as a flex catheter), a balloon catheter 16 extending through the guide catheter 14, and a nose catheter 18 extending through the balloon catheter 16. The guide catheter 14, the balloon catheter 16, and the nose catheter 18 in the illustrated aspect are adapted to slide longitudinally relative to each other to facilitate delivery and positioning of the valve (prosthetic device) 12 at an implantation site in a patient's body, as described in detail below. Generally, sheath 8 is inserted into a vessel, such as the transfemoral vessel, passing through the skin of the patient, such that the distal end of the sheath 8 is inserted into the vessel. Sheath 8 can include a hemostasis valve at the opposite, proximal end of the sheath. The delivery apparatus 10 can be inserted into the sheath 8, and the prosthetic device 12 can then be delivered and implanted within the patient.

The present disclosure relates to various configurations of the sheath. These exemplary aspects are directed to a sheath for delivering a medical device, wherein the sheath has a proximal and a distal end and comprises an elongated tube forming an outer layer of the sheath that is positioned at at least the proximal end of the sheath and extending along at least a portion of a length of the sheath, having an inner surface and an outer surface, and wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof; less than about 65% of an inorganic filler based on a total weight of the first compound composition; and up to about 20% of a solid lubricant filler based on a total weight of the first compound composition; wherein the elongated tube is configured to reversibly expand from an initial diameter do in an unexpended position to an expanded diameter dₑ in an expanded position upon passage of a medical device; and wherein the sheath exhibits at least a 10% reduction in an insertion force when compared with a substantially identical reference sheath that does not comprise the first polymer layer.

It is understood, however, that also disclosed are aspects where the disclosed sheaths can comprise additional components. These exemplary aspects are disclosed herein, as shown below in detail.

In certain aspects, the elongated tube comprises a first polymer layer. In such exemplary aspects, the first polymer layer can comprise a first compound composition comprising from greater than 0 wt % to less than 100 wt %, including exemplary values of about 0.01 wt %, about 1wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, and about 99.9 wt % of a polymer comprising a polyether block amide, a polyurethane, or any combination thereof.

In still further aspects, the first compound composition can comprise from greater than about 35 wt % to less than about 80 wt %, including exemplary values of about 40 wt %, about 45 wt %, about 50 wt %, about 55 wt %, about 60 wt %, about 65 wt %, about 70 wt %, and about 75 wt % of a polymer comprising a polyether block amide, a polyurethane, or any combination thereof.

In certain aspects, the polymer in the first compound composition comprises a polyether block amide. In such exemplary aspects, the polyether block amide can comprise PEBAX® from Arkema. In yet further aspects, the polymer can comprise polyurethane, for example, NEUSoft®. While in still further aspects, the polymer can compromise a combination of the polyether block amide, such as, for example, PEBAX® and polyurethane. It is further understood that if the mixture of the polymers is present, such a mixture can comprise each component in any amount relative to another component to provide the desired polymer falling within the disclosed above range.

In still further aspects, the first compound composition can comprise less than about 65 wt % of an inorganic filler based on a total weight of the first compound composition, including exemplary values of less than about 60 wt %, less than about 55 wt %, less than about 50 wt %, less than about 45 wt %, less than about 40 wt %, less than about 35 wt %, less than about 30 wt %, less than about 25 wt %, less than about 20 wt %, less than about 15 wt %, less than about 10 wt %, less than about 5 wt %, and less than about 1wt % of the inorganic filler.

In yet further aspects, the inorganic filler can be present in an amount of at least about 1wt %, at least about 2 wt %, at least about 5 wt %, at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, or at least about 55 wt %.

In still further aspects, the inorganic filler can comprise any inorganic materials that can be used as a filler and are acceptable for the desired application. In certain exemplary and unlimiting aspects, the inorganic filler can comprise bismuth oxychloride, barium sulfate, bismuth subcarbonate, calcium carbonate, aluminum trihydrate, barite, kaolin clay, limestone, or any combination thereof. Again it is understood that the inorganic filler can comprise a combination of the various fillers. In such exemplary aspects, an amount of each filler in the combination can be in any range to provide a final combination that falls within the disclosed above range.

In still further aspects, the first compound composition can comprise up to about 20 wt % of a solid lubricant filler based on a total weight of the first compound composition, including exemplary values of about 0.01 wt %, about 0.1 wt %, about 0.5 wt %, about 1wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, and about 19.9 wt %. In yet further aspects, the solid lubricant filler can be present up to about 20 wt %, up to about 15 wt %, or up to about 10 wt % based on a total weight of the first compound composition.

In still further aspects, the solid lubricant filler can comprise any additive that is known to reduce friction and behave as a lubricant. In such exemplary and unlimiting aspects, the solid lubricant filler can comprise one or more of graphene, reduced graphene oxide, carbon black, boron nitride, silicones, talc, polytetrafluorethylene (PTFE), fluorinated ethylene propylene, and the like. In still further aspects, the solid lubricant comprises a PTFE filler. In yet further aspects, the PTFE filler is a powder.

In still further aspects, the first compound composition can further comprise at least one tackiness reducing compound. Any compounds known in the art as capable of reducing the tackiness of the polymer composition can be considered and used for the purpose of this disclosure. In yet further exemplary and unlimiting aspects, the at least one tackiness reducing compound comprises ProPell™ from Foster Corporation

In certain aspects, the at least one tackiness reducing compound is present in an amount from 0 wt % to about 20 wt %, including exemplary values of about 0.01 wt %, about 0.05 wt %, about 0.1 wt %, about 0.5 wt %, about 1wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, and about 19 wt % based on a total weight of the first compound composition. In still further aspects, the at least one tackiness reducing compound is present in any amount having a value between any two foregoing values. For example and without limitation, the at least one tackiness reducing compound can be present in an amount from about 1wt % to about 5 wt %, or from about 5 wt % to about 10 wt % based on a total weight of the first compound composition.

In still further aspects and as disclosed herein, the first polymer has a substantially same durometer along a total length of the elongated tube. It is understood, however, the durometer of the first polymer of the elongated tube can also be varied along the length of the tube. For example, and without limitation, disclosed herein are aspects where a durometer of the first polymer at a proximal end of the elongated tube is different from a durometer of the first polymer at a distal end of the elongated tube.

In still further aspects, the first polymer in the first polymer layer has a Shore D from about 20 D to about 72 D, including exemplary values of about 25 D, about 30 D, about 35 D, about 40 D, about 45 D, about 50 D, about 55 D, about 60 D, about 65 D, and about 70 D. In still further aspects, the first polymer in the first polymer layer has a Shore D from about 20 D to about 35 D. In still further aspects, the first polymer in the first polymer layer has a Shore D of about 30 D. Yet, in still further aspects, the first polymer in the first polymer layer has a Shore D of about 25 D.

It is understood that the elongated tube, as disclosed herein, can comprise aspects where only one polymer layer is present. Yet, in other aspects, two or more polymer layers can be present in the elongated tube. In such exemplary aspects, the elongated tube comprises at least a second polymer layer comprising a second compound composition comprising from greater than 0 wt % to 100 wt % of a second polymer comprising polyether block amide, a polyurethane, or a composition thereof. Similar to the first compound composition, the second polymer can be present in any amount that falls within the disclosed range. For example, the second polymer can be present in the second compound composition from greater than 0 wt %, about 0.01 wt %, about 1wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, and about 99.9 wt % of a polymer comprising a polyether block amide, a polyurethane, or any combination thereof. In yet further aspects, the second polymer can be present in the second compound composition from greater than about 95 wt % to less than about 99 wt %, including exemplary values of about 95.5 wt %, about 96 wt %, 96.5 wt %, about 97 wt %, about 97.5 wt %, about 98 wt %, and about 98.5 wt %.

In yet further aspects, the second compound composition can further comprise up to 20 wt % of a tackiness reducing additive, including exemplary values of about 0.01 wt %, about 0.05 wt %, about 0.1 wt %, about 0.5 wt %, about 1wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, and about 19 wt % based on a total weight of the second compound composition. In still further aspects, the at least one tackiness reducing compound is present in any amount having a value between any two foregoing values. For example and without limitation, the at least one tackiness reducing compound can be present in an amount from about 1wt % to about 5 wt %, or from about 5 wt % to about 10 wt % based on a total weight of the second compound composition. In still further aspects and as disclosed herein, the second compound composition can be substantially free of a solid lubricant filler.

It is further understood that in certain aspects, the first polymer in the first compound composition can be the same as the second polymer in the second compound composition. Yet, in other aspects, the first polymer in the first compound composition is different from the second polymer in the second compound composition. In yet further aspects, the second polymer layer comprises PEBAX®. While in further aspects, the second polymer layer can comprise polyurethane, for example, NEUSoft® from PolyOne.

In still further aspects, the second polymer has a Shore D from about 20 D to about 35 D. Yet, in further aspects, the second polymer has a Shore D of about 25 D or about 35 D.

In still further aspects, the second compound composition can be substantially free of an inorganic filler. While in certain aspects, the inorganic filler can be present in the second compound composition in any amount from greater than 0 wt % to less than 100 wt %, including exemplary values of about 0.01 wt %, about 0.05 wt %, about 0.1 wt %, about 0.5 wt %, about 1wt %, about 5 wt %, about 10 wt %, about 20 wt %, about 30 wt %, about 40 wt %, about 50 wt %, about 60 wt %, about 70 wt %, about 80 wt %, about 90 wt %, and about 95 wt %. In the aspects where the inorganic filler is present in the second compound composition, a such inorganic filler can comprise any filler disclosed above.

In still further aspects, and as disclosed herein, the elongated tube has a predetermined thickness, and wherein at least about 10%, at least about 20%, at least about 30%, at least about 40%, at least about 50%, at least about 60%, at least about 70%, at least about 80%, at least about 90%, or 100% of the predetermined thickness comprises the first and/or the second compound composition comprising the first and/or the second polymer having a Shore D equal to or lower than about 30 D.

In still further aspects, the predetermined thickness of the elongated tube can vary along a length of the sheath. While in other aspects, the predetermined thickness of the elongated tube is the same along a length of the sheath. Yet, in further aspects, the predetermined thickness of the elongated tube is greater at the proximal end. In still further aspects, the predetermined thickness of the elongated tube is up to 6 mils, for example, and without limitation from about 1 mil to about 6 mils, including exemplary values of about 1.5 mils, about 2 mils, about 2.5 mils, about 3 mils, about 3.5 mils, about 4 mils, about 4.5 mils, about 5 mils, about 5.5 mils, and about 5.9 mils.

In still further aspects, the first polymer layer and the second polymer layer can have the same thickness. While in other aspects, the first polymer layer and the second polymer layer have different thicknesses. For example, in some aspects, the first polymer layer has a thickness of about 1 mil to about 5 mils, including exemplary values of about 1.1 mils, about 1.2 mils, about 1.3 mils, about 1.4 mils, about 1.5 mils, about 1.6 mils, about 1.7 mils, about 1.8 mils, about 1.9 mils, about 2.0 mils, 2.1 mils, about 2.2 mils, about 2.3 mils, about 2.4 mils, about 2.5 mils, about 2.6 mils, about 2.7 mils, about 2.8 mils, about 2.9 mils, about 3.0 mils, about 3.1 mils, about 3.2 mils, about 3.3 mils, about 3.4 mils, about 3.5 mils, about 3.6 mils, about 3.7 mils, about 3.8 mils, about 3.9 mils, about 4.1 mils, about 4.2 mils, about 4.3 mils, about 4.4 mils, about 4.5 mils, about 4.6 mils, about 4.7 mils, about 4.8 mils, and about 4.9 mils. Yet still, in further aspects, the second polymer layer can have a thickness of about 2 mils to about 6 mils, including exemplary values of 2.1 mils, about 2.2 mils, about 2.3 mils, about 2.4 mils, about 2.5 mils, about 2.6 mils, about 2.7 mils, about 2.8 mils, about 2.9 mils, about 3.0 mils, 3.1 mils, about 3.2 mils, about 3.3 mils, about 3.4 mils, about 3.5 mils, about 3.6 mils, about 3.7 mils, about 3.8 mils, about 3.9 mils, about 4.0 mils, about 4.1 mils, about 4.2 mils, about 4.3 mils, about 4.4 mils, about 4.5 mils, about 4.6 mils, about 4.7 mils, about 4.8 mils, about 4.9 mils, about 5.1 mils, about 5.2 mils, about 5.3 mils, about 5.4 mils, about 5.5 mils, about 5.6 mils, about 5.7 mils, about 5.8 mils, and about 5.9 mils.

In still further aspects, the predetermined thickness of the elongated tube is greater at the proximal end. While in other aspects, the predetermined thickness of the elongated tube is smaller at the distal end as compared to the predetermined thickness of the elongated tube at the proximal end.

In still further aspects where two or more layers are present in the elongated tube, the first polymer layer can define the inner surface of the elongated tube, while the second polymer layer can define the outer surface of the elongated tube. However, there are also aspects where the first polymer layer defines the outer surface of the elongated tube, while the second polymer layer defines the inner surface of the elongated tube. It is also understood that other aspects are also enclosed, where one or more additional polymer layers are disposed between the first polymer layer and the second polymer layer.

In still further aspects, the elongated tube can be extruded. In the aspects where the first and the second polymer layers are present, such polymer layers can be co-extruded. In still further aspects, the first polymer layer can be substantially bonded to the second polymer layer. In such exemplary aspects, the first polymer layer substantially does not delaminate from the second polymer layer. It is understood that in some aspects, the bonding can be physical or chemical or any other type known in the art.

In still further aspects, any sheath that comprises the disclosed herein elongated tube can exhibit at least about 10% reduction, at least about 15% reduction, at least 20% reduction, at least about 25% reduction, at least about 30% reduction, at least about 35% reduction, at least about 40% reduction, at least about 45% reduction, or at least about 50% reduction in an insertion force when compared with a substantially identical reference sheath that does not comprise the first polymer layer.

In still further aspects, any sheath that comprises the disclosed herein elongated tube can exhibit an insertion force of less than about 55 N, less than about 50 N, less than about 45 N, less than about 40 N, less than about 35 N, or less than about 35 N when a medical device is pushed through the sheath.

In still further aspects, the elongated tube can also exhibit a friction force of less than about 10 N, or less than about 9 N, or less than about 8 N, or less than about 7 N, or less than about 6 N, or even less than about 5 N, in the dry state against a substrate surface comprising one or more of polytetrafluoroethylene, fluorinated ethylene propylene, or high-density polyethylene having a diameter of about 0.300″.

In still further aspects, the elongated tube can exhibit a hoop force at 10 mm extension (about 85% strain) of less than about 10 N, or less than about 9 N., or less than about 8 N, or less than about 7 N, or less than about 6 N, or even less than about 5 N. In such exemplary aspects, the elongated tube can have a diameter of about 0.290″ (7.4 mm) and wall thickness, as disclosed herein. In aspects where the elongated tube has a diameter of about 0.290″ (7.4 mm) and a total wall thickness of about 0.0045″, with a sample length of about 0.25″ (6.4 mm), a hoop direction forces at 10 mm extension can be less than about 8 N. It is understood that in some exemplary and unlimiting aspects, a low force at 10 mm extension is desired for low sheath expansion force.

In still further aspects, the elongated tube can exhibit an elongation at break of ranging between about 650% and about 800%, including exemplary values of about 680%, about 700%, about 710%, about 750%, and about 780%. It is understood that in some exemplary and unlimiting aspects, a high elongation is preferable for expansion to a larger diameter before the elongated tube breaks.

In still further aspects, the elongated tube is substantially kink resistant.

In certain aspects, the elongated tube extends along a portion of the length of the sheath. In such exemplary aspects, the elongated tube can be positioned at the proximal end of the sheath, or in the middle of the sheath, or at the distal portion of the sheath. While in other aspects, the elongated tube extends along the whole length of the sheath. In such exemplary aspects, the elongated tube can be positioned at the proximal end of the sheath and extend to the distal end of the sheath.

In still further aspects, the sheath can further comprise an expandable tubular inner liner extending along the length of the sheath and comprising at least one folded portion, wherein the expandable inner liner has an inner surface and an outer surface, wherein the inner surface of the expandable inner liner defines a lumen and forms an inner surface of the at least one folded portion, and wherein the outer surface extends circumferentially to form an outer surface of the at least one folded portion; and a first outer tubular layer extending at least partially along the length of the sheath and having an inner surface and an outer surface, wherein the inner surface of the first outer tubular layer further extends at least partially around the outer surface of the inner liner such that at least a portion of the inner surface of the first outer tubular layer is positioned adjacent to the outer surface of the at least one folded portion of the inner liner; wherein the elongated tube is positioned such that at least a portion of the inner surface of the elongated tube overlies at least a portion of the outer surface of the first outer tubular layer.

In still further aspects, the elongated tube disclosed herein can comprise at least two polymer layers. In still further aspects, the elongated tube disclosed herein can comprise at least one intermediate reinforcement layer disposed between the first polymer layer and the second polymer layer. In still further aspects, the at least one intermediate reinforcement layer is a polymer layer.

In some aspects, the at least one intermediate layer can extend along the whole circumference of the elongated tube. In yet further aspects where the first polymer layer forms the inner surface of the elongated tube and the second polymer layer forms the outer surface of the elongated tube, the intermediate layer is disposed between the outer surface of the first polymer and the inner surface of the second polymer layer. Yet in other aspects, and as disclosed above, if the second polymer layer forms the inner surface of the elongated tube and the first polymer layer forms the outer surface of the elongated tube, the intermediate layer is disposed between the outer surface of the second polymer layer and the inner surface of the first polymer layer. In still further aspects, the intermediate reinforcement layer can bond the first and second polymer layers and can also assist in bonding the elongated tubing as a whole to an inner member of the sheath.

In still further aspects, the at least one intermediate layer has a finite width that is smaller than the circumference of the elongated tube. In such aspects, the at least one intermediate layer can be inserted as a strip between the first and the second polymer layers. In some exemplary and unlimiting aspects, if the elongated tube has a distal outer diameter of about 0.200″, the strip can have a width between about 0.010″ to about 0.150″, including exemplary values of about 0.03″, about 0.035″, about 0.04″, about 0.045″, about 0.05″, about 0.055″, about 0.06″, about 0.065″, about 0.07″, about 0.075″, about 0.08″, about 0.085″, about 0.09″, about 0.095″, about 0.10″, about 0.105″, about 0.110″, about 0.115″, about 0.120″, about 0.125″, about 0.130″, about 0.135″, about 0.140″, and about 0.145″. It is understood that the widths shown above are exemplary, and if the distal outer diameter of the elongated sheath has a size different from 0.200″, the strip width can be adjusted in the same or a different ratio.

In still further aspects, the at least one intermediate layer has a finite width that is smaller than the circumference of the elongated tube. In such aspects, the at least one intermediate layer can be inserted as a strip between the first and the second polymer layers. In some exemplary and unlimiting aspects, if the elongated tube has a distal outer diameter of about 0.200″, the strip can have a width between about 5% to about 50% of the circumference of the elongated tube. In still further aspects, the total combined width of the strips is about 5%, about 15%, about 20%, about 25%, about 30%, about 35%, about 40%, about 45%, or about 50% of the circumference of the elongated tube. It is understood that the widths shown above are exemplary, and if the distal outer diameter of the elongated sheath has a size different from 0.200″, the strip width can be adjusted in the same or a different ratio.

In still further aspects, the elongated tube can comprise two or more intermediate layers. In such aspects, the two or more intermediate layers can be disposed, as individual strips, circumferentially between the first and the second polymer layers at a predetermined distance from each other. In aspects where the two or more intermediate layers are disposed between the first and the second polymer layers of the elongated tube, a total combined width of all the strips is about 5% to about 50% of the circumference of the elongated tube. In still further aspects, the total combined width of the strips is about 5%, about 15%, about 20%, about 25%, about 30%, about 35%, about 40%, about 45%, or about 50% of the circumference of the elongated tube.

In still further aspects, the at least one intermediate layer is configured to provide an axial reinforcement to the elongated tube and, as a result, to the sheath where the elongated tube can be used. In such exemplary aspects, the at least one intermediate layer can be disposed along the length of the elongated tube or along a portion of the length of the elongated tube.

In some aspects, the portion of the length of the elongated tube where the at least one intermediate layer is disposed is positioned at the distal end and/or proximal end of the elongated tube. In yet other aspects, the at least one intermediate layer can also be positioned anywhere along the length of the elongated tube.

It is further understood that in the aspects where the intermediate layer is present as one or more strips disposed circumferentially along the length of the sheath, the width of the strip can be the same along the length, or it can vary along the length. In aspects where the strips' width varies along the length of the elongated tube, such a strip can have any of the disclosed above width values.

In still further aspects, the first polymer layer used in this exemplary elongated tube can be any of the first polymer layers described above. In still further exemplary and unlimiting aspects, the first polymer layer forms the inner surface of the elongated tube and comprises a first compound composition comprising from greater than 0 wt % to less than 100 wt % of a first polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition.

Any of the disclosed above inorganic fillers, and solid lubricant fillers can be present in any amount as disclosed. For example, the inorganic filler can comprise bismuth oxychloride, barium sulfate, bismuth subcarbonate, calcium carbonate, aluminum trihydrate, barite, kaolin clay, limestone, or any combination thereof. In yet other aspects, the inorganic filler can be present in at least 10 wt %. In still further aspects, the inorganic filler can be present in an amount of less than about 50 wt % based on a total weight of the first compound composition.

In yet further aspects, the solid lubricant filler can comprise a PTFE filler.

The first compound can also comprise any of the disclosed above additives. For example, the compound can comprise at least one tackiness reducing compound in an amount from about 1wt % to about 20 wt %.

In still further exemplary aspects, the polymer present in the first compound can have Shore D from about 20 D to about 35 D, including exemplary values of about 22 D, about 25 D, about 27 D, about 30 D, and about 32 D.

In yet further aspects, a durometer of the first polymer in the first polymer layer at a proximal end of the elongated tube can be different from a durometer of the first polymer in the first polymer layer at a distal end of the elongated tube.

In still further aspects, the polymer in the first compound can comprise poly ether block amide, for example, PEBAX®. While in other aspects, the polymer in the first compound can comprise polyurethane. In still further aspects, the first compound can also comprise polyamide.

In still further aspects, the thickness of the first polymer layer can be from about 1 mil to about 5 mils, including exemplary values of about 1.1 mils, about 1.2 mils, about 1.3 mils, about 1.4 mils, about 1.5 mils, about 1.6 mils, about 1.7 mils, about 1.8 mils, about 1.9 mils, about 2.0 mils, 2.1 mils, about 2.2 mils, about 2.3 mils, about 2.4 mils, about 2.5 mils, about 2.6 mils, about 2.7 mils, about 2.8 mils, about 2.9 mils, about 3.0 mils, about 3.1 mils, about 3.2 mils, about 3.3 mils, about 3.4 mils, about 3.5 mils, about 3.6 mils, about 3.7 mils, about 3.8 mils, about 3.9 mils, about 4.1 mils, about 4.2 mils, about 4.3 mils, about 4.4 mils, about 4.5 mils, about 4.6 mils, about 4.7 mils, about 4.8 mils, and about 4.9 mils.

In still further aspects, the second polymer layer can comprise any of the disclosed above polymers. In some aspects, the second polymer layer can comprise a second compound composition comprising from greater than 0 wt % to 100 wt % of a second polymer comprising polyether block amide, a polyurethane, or a composition thereof. In still further aspects, the second polymer layer can comprise a polyamide. In yet some other aspects, the second compound can also comprise any of the fillers or additives disclosed above. While in some aspects, the second compound does not comprise the solid lubricant fillers disclosed herein. While in still further aspects, the second compound can comprise a tackiness reducing additive described in this disclosure. In some aspects, the second polymer can be a polyurethane. In still further aspects, the polyurethane is a thermoplastic polyurethane. While in still further aspects, the second polymer can be a blend comprising a polyurethane with a styrene block copolymer. In still further aspects, the blend can further comprise additional polymers and copolymers. For example, ether-based polymers can be present in the blend. In some exemplary and unlimiting aspects, the second polymer can be chosen from commercially available polymers sold under the trade name of Neusoft™. In still further aspects, the second polymer can have a Shore A durometer from about 20 A to about 75 A, including exemplary values of about 25 A, about 30 A, about 35 A, about 40 A, about 45 A, about 50 A, about 55 A, about 60 A, about 65 A, and about 70 A. In yet further aspects, the second polymer can have a Shore A durometer of less than 60 A. In some exemplary aspects, the second polymer can be Neusoft™ 597-50A.

In still further aspects, the thickness of the second polymer layer can be from about 1 mil to about 6 mils, including exemplary values of about 1.1 mils, about 1.2 mils, about 1.3 mils, about 1.4 mils, about 1.5 mils, about 1.6 mils, about 1.7 mils, about 1.8 mils, about 1.9 mils, about 2.0 mils, 2.1 mils, about 2.2 mils, about 2.3 mils, about 2.4 mils, about 2.5 mils, about 2.6 mils, about 2.7 mils, about 2.8 mils, about 2.9 mils, about 3.0 mils, about 3.1 mils, about 3.2 mils, about 3.3 mils, about 3.4 mils, about 3.5 mils, about 3.6 mils, about 3.7 mils, about 3.8 mils, about 3.9 mils, about 4.1 mils, about 4.2 mils, about 4.3 mils, about 4.4 mils, about 4.5 mils, about 4.6 mils, about 4.7 mils, about 4.8 mils, about 4.9 mils, about 5.1 mils, about 5.2 mils, about 5.3 mils, about 5.4 mils, about 5.5 mils, about 5.6 mils, about 5.7 mils, about 5.8 mils, and about 5.9 mils. In still further aspects, the thickness of the at least one intermediate reinforcement layer can be anywhere between about 1 mil to about 6 mils, including exemplary values of about 1.1 mils, about 1.2 mils, about 1.3 mils, about 1.4 mils, about 1.5 mils, about 1.6 mils, about 1.7 mils, about 1.8 mils, about 1.9 mils, about 2.0 mils, 2.1 mils, about 2.2 mils, about 2.3 mils, about 2.4 mils, about 2.5 mils, about 2.6 mils, about 2.7 mils, about 2.8 mils, about 2.9 mils, about 3.0 mils, about 3.1 mils, about 3.2 mils, about 3.3 mils, about 3.4 mils, about 3.5 mils, about 3.6 mils, about 3.7 mils, about 3.8 mils, about 3.9 mils, about 4.1 mils, about 4.2 mils, about 4.3 mils, about 4.4 mils, about 4.5 mils, about 4.6 mils, about 4.7 mils, about 4.8 mils, about 4.9 mils, about 5.1 mils, about 5.2 mils, about 5.3 mils, about 5.4 mils, about 5.5 mils, about 5.6 mils, about 5.7 mils, about 5.8 mils, and about 5.9 mils.

In still further aspects, the at least one intermediate layer can comprise any of the polymers disclosed herein. In some aspects, the at least one intermediate layer can comprise the first compound disclosed above. Yet, in other aspects, the at least one intermediate layer can comprise the second compound disclosed above. While in still further aspects, the at least one intermediate layer can comprise the first compound. Yet, in still further aspects, the at least one intermediate layer can comprise any polymers that are known in the art and suitable for the desired application. In some aspects, the at least one intermediate layer can comprise polyether block amide, polyurethane, or a combination thereof. While in still further aspects, the at least one intermediate layer is a polyether block amide, for example, PEBAX®. While in still further aspects, the intermediate layer is a polyurethane. In such exemplary aspects, the at least one intermediate layer does not comprise a solid lubricant filler, such as a PTFE. In yet other aspects, the at least one intermediate layer does not comprise an inorganic filler. In still further aspects, the at least one intermediate layer can comprise a polymer comprising PEBAX® or polyurethane having a Shore D (or Shore A) durometer between about 45 D (85A) to about 90 D, including exemplary values of about 50 D, about 55 D, about 60 D, about 65 D, about 70 D, about 72 D, about 75 D, about 80 D, and about 85 D.

In yet further aspects, the at least one intermediate reinforcement layer can comprise a polyolefin. In still further aspects, the at least one intermediate reinforcement layer can comprise a polyethylene, a polypropylene, a graft modified polyethylene or polypropylene. In yet further aspects, the at least one intermediate reinforcement layer can comprise the grafted low-density polyethylene (LDPE), grafted medium density polyethylene, grafted ultra-low-density polyethylene (ULDPE), grafted high density polyethylene (HDPE), grafted heterogeneously branched linear low-density polyethylene (LLDPE), grafted homogeneously branched linear ethylene polymers and substantially linear ethylene polymers, grafted polypropylene, or ethylene vinyl acetate (EVA), or any combination thereof. In such exemplary aspects, a maleic anhydride or an acrylic acid can be used to graft the disclosed above polymers. In still further aspects, the at least one intermediate reinforcement layer can comprise a maleic anhydride or an acrylic acid grafted low-density polyethylene. In yet further aspects, the at least one intermediate reinforcement layer can comprise a maleic anhydride or an acrylic acid grafted polypropylene. In still further aspects, the at least one intermediate reinforcement layer can comprise a maleic anhydride or an acrylic acid grafted ethylene vinyl acetate. In still further aspects, the at least one intermediate reinforcement layer can comprise a maleic anhydride grafted polyolefin sold under a trademark of OREVAC®.

In still further aspects, any of the disclosed above at least one intermediate reinforcement layer can thermally bond the elongated tube to the inner member of the sheath. In still further aspects, the intermediate reinforcement layer can be extruded to be positioned between the first polymer layer and the second polymer layer. In still further aspects, the at least one intermediate reinforcement layer can be fused with the first and second polymer layers by at least one of heat or compression.

In still further aspects, the elongated tube, as disclosed herein, comprising the at least one intermediate reinforcement layer can exhibit an expansion force of less than about 50 N, less than about 49N, less than about 48N, less than about 47N, less than about 46N, less than about 45N, less than about 44N, less than about 43N, less than about 42N, less than about 41N, or even less than about 40 N.

In still further aspects, the elongated tube as disclosed herein that comprises the at least one intermediate reinforcement layer can exhibit a burst pressure greater than about 8 psi, greater than about 8.5 psi, greater than about 9 psi, greater than about 9.5 psi, greater than about 10 psi, greater than about 10.5 psi, greater than about 11 psi, greater than about 11.5 psi, about 12 psi, greater than about 12.5 psi, greater than about 13 psi, greater than about 13.5 psi, greater than about 14 psi, greater than about 14.5 psi, or greater than about 15 psi.

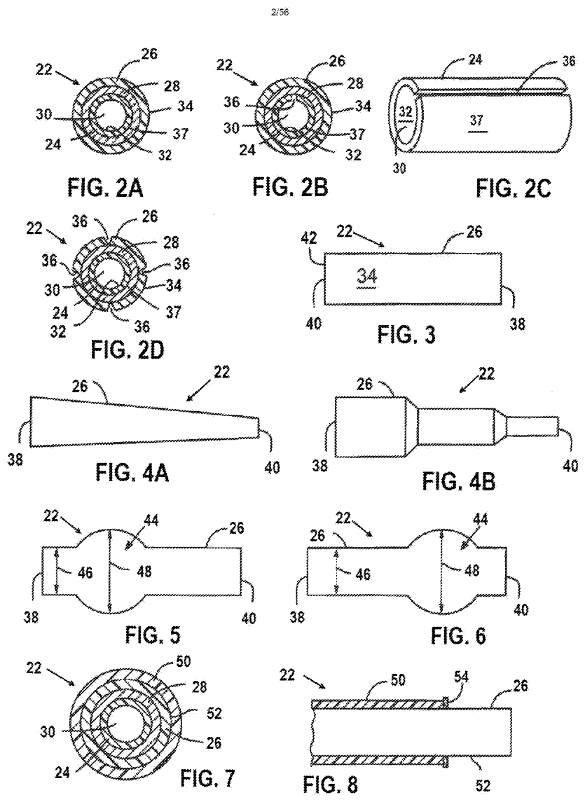

FIGS. 2A, 2B, and 2D show section views of aspects of a sheath 22 for use with a delivery apparatus such as that shown in FIG. 1. Example expandable sheaths are also disclosed in U.S. patent application Ser. No. 12/249,867, filed Oct. 10, 2008 (now U.S. Pat. No. 8,690,936), U.S. patent application Ser. No. 13/312,739, filed Dec. 6, 2011 (now U.S. Pat. No. 8,790,387), U.S. patent application Ser. No. 14/248,120 filed on Apr. 8, 2014 (now U.S. Pat. No. 9,301,840), U.S. patent application Ser. No. 14/324,894, filed Jul. 7, 2014 (now U.S. Pat. No. 9,301,841), U.S. patent application Ser. No. 15/057,953, filed Mar. 1, 2016 (now U.S. Pat. No. 9,987,134), U.S. patent application Ser. No. 15/997,587, filed Jun. 4, 2018, U.S. patent application Ser. No. 16/149,953, filed on Oct. 2, 2018 (now U.S. Pat. No. 10,524,905), U.S. patent application No. Ser. 16/149,956, filed on Oct. 2, 2018 (now U.S. Pat. No. 10,517,720), U.S. patent application Ser. No. 16/149,960, filed on Oct. 2, 2018 (now U.S. Pat. No. 10,524,906, and U.S. patent application Ser. No. 16/149,969, filed on Oct. 2, 2018 (now U.S. Pat. No. 10,524,907), the disclosures of which are herein incorporated by reference.

FIG. 2C shows a perspective view of one aspect of an inner layer (or tubular inner liner) 24 for use with the sheath 22. Sheath 22 includes an inner layer, such as inner polymeric tubular liner 24, an outer layer, such as the outer polymeric tubular layer 26. Sheath can also include an intermediate tubular layer 28 disposed between the inner and outer polymeric tubular layers (liner) 24, 26. The sheath 22 defines a lumen 30 through which a delivery apparatus can travel into a patient's vessel in order to deliver, remove, repair, and/or replace a prosthetic device. Such introducer sheaths 22 can also be useful for other types of minimally invasive surgery, such as any surgery requiring introduction of an apparatus into a subject's vessel. For example, the sheath 22 also can be used to introduce other types of delivery apparatus for placing various types of intraluminal devices (e.g., stents, stented grafts, etc.) into many types of vascular and non-vascular body lumens (e.g., veins, arteries, esophagus, ducts of the biliary tree, intestine, urethra, fallopian tube, other endocrine or exocrine ducts, etc.).

The outer polymeric tubular layer 26 and the inner polymeric tubular liner 24 can comprise, for example, polytetrafluoroethylene (PTFE) (e.g., Teflon®), polyimide, PEEK, polyurethane, nylon, polyethylene, polyamide, polyether block amides (e.g., PEBAX®), polyether block ester copolymer, polyesters, fluoropolymers, polyvinyl chloride, thermoset silicone, latex, poly-isoprene rubbers, polyolefin, other medical grade polymers, or combinations thereof. In yet other aspects, the outer tubular layer can also comprise a high-density polyethylene (HDPE).

The intermediate tubular layer 28 can comprise a shape memory alloy such as Nitinol and/or stainless steel, cobalt chromium, spectra fiber, polyethylene fiber, aramid fiber, or combinations thereof.

The inner polymeric tubular liner 24 can advantageously be provided with a low coefficient of friction on its inner surface. For example, the inner polymeric tubular liner 24 can have a coefficient of friction of less than about 0.5, less than about 0.1, less than about 0.05, or even less than about 0.01. Some aspects of a sheath 22 can include an additional lubricious liner on the inner surface 32 of the inner polymeric tubular liner 24. Such a liner can facilitate the passage of a delivery apparatus through the lumen 30 of the sheath 22. Examples of suitable lubricious liners include materials that can reduce the coefficient of friction of the inner polymeric tubular liner 24, such as PTFE, polyethylene, polyvinylidene fluoride, and combinations thereof. Suitable materials for a lubricious liner also include other materials desirably having a coefficient of friction of less than about 0.5, less than about 0.1, less than about 0.05, or even less than about 0.01.

The inner diameter of the intermediate tubular layer 28 varies depending on the application and size of the delivery apparatus and prosthetic device. In some aspects, the inner diameter ranges from about 0.005 inches to about 0.400 inches, including exemplary values of about 0.01 inches, about 0.05 inches, about 0.100 inches, about 0.120 inches, about 0.150 inches, about 0.170 inches, about 0.200 inches, about 0.220 inches, about 0.250 inches, about 0.270 inches, about 0.300 inches, bout 0.320 inches, about 0.350 inches, and about 0.370 inches.

The thickness of the intermediate tubular layer 28 can be varied depending on the desired amount of radial expansion, as well as the strength required. For example, the thickness of the intermediate tubular layer 28 can be from about 0.002 inches to about 0.0025 inches, including exemplary values of about 0.003 inches, about 0.004 inches, about 0.005 inches, about 0.006 inches, about 0.007 inches, about 0.008 inches, 0.009 inches, about 0.010 inches, about 0.011 inches, about 0.012 inches, about 0.013 inches, about 0.014 inches, about 0.015 inches, about 0.016 inches, about 0.017 inches, about 0.018 inches, 0.019 inches, about 0.020 inches, about 0.021 inches, about 0.022 inches, about 0.023 inches, and about 0.024 inches.

The thicknesses of the inner polymeric tubular liner 24 and the outer polymeric tubular layer 26 can also be varied depending on the particular application of the sheath 22. In some aspects, the thickness of the inner polymeric tubular liner 24 ranges from about 0.0005 inches to about 0.010 inches, including exemplary values of about 0.0006 inches, about 0.0007 inches, about 0.0008 inches, about 0.0009 inches, about 0.001 inches, about 0.002 inches, 0.003 inches, about 0.004 inches, about 0.005 inches, about 0.006 inches, about 0.007 inches, about 0.008 inches, and about 0.009 inches. In one aspect, the thickness can be about 0.002 inches. Outer polymeric tubular layers 26 can have a thickness of from about 0.002 inches to about 0.015 inches, including exemplary values of about 0.003 inches, about 0.004 inches, about 0.005 inches, about 0.006 inches, about 0.007 inches, about 0.008 inches, 0.009 inches, about 0.010 inches, about 0.011 inches, about 0.012 inches, about 0.013 inches, and about 0.014 inches. In certain aspects, the outer polymeric tubular layer 26 can have a thickness of about 0.010 inches.

The hardness of each layer of the sheath 22 can also be varied depending on the particular application and desired properties of the sheath 22. In some aspects, the outer polymeric tubular layer 26 has a Shore D durometer of about 25 D to about 75 D.

Additionally, some aspects of a sheath 22 can include an exterior hydrophilic coating on the outer surface 34 of the outer polymeric tubular layer 26. Such a hydrophilic coating can facilitate the insertion of the sheath 22 into a patient's vessel. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V., Heerlen, the Netherlands), as well as other hydrophilic coatings, are also suitable for use with the sheath 22.

In some aspects, the outer surface 34 of the outer polymeric tubular layer 26 can be modified. For example, surface modifications such as plasma etching can be performed on the outer surface 34. Similarly, other surfaces, both outer and inner, can be surface modified according to certain aspects and desired applications. In some aspects, surface modification can improve adhesion between the layers in the areas of the modification.

In certain aspects, the outer surface of the first outer tubular layer can be at least partially etched. In yet still, further aspects, the outer surface of the inner liner can be selectively etched around the circumference, linearly along at least a portion of the length of the sheath, or a combination thereof.

The sheath 22 also can have at least one radiopaque filler or marker. The radiopaque filler or marker can be associated with the outer surface 34 of the outer polymeric tubular layer 26. Alternatively, the radiopaque filler or marker can be embedded or blended within the outer polymeric tubular layers 26. Similarly, the radiopaque filler or marker can be associated with a surface of the inner polymeric tubular liner 24 or the intermediate tubular layer 28 or embedded within either or both of those layers.

Suitable materials for use as a radiopaque filler or marker include, for example, barium sulfite, bismuth trioxide, titanium dioxide, bismuth subcarbonate, or combinations thereof. The radiopaque filler can be mixed with or embedded in the material used to form the outer polymeric tubular layer 26 and can comprise from about 5% to about 45% by weight, including exemplary values of about 10% by weight, about 15% by weight, about 20% by weight, about 30% by weight, about 35% by weight, and about 40% by weight of the outer polymeric tubular layer. More or less radiopaque material can be used in some aspects, depending on the particular application.

In some aspects, the inner polymeric tubular liner 24 can comprise a substantially uniform cylindrical tube. In alternative aspects, the inner polymeric tubular liner 24 can have at least one section of discontinuity along its longitudinal axis to facilitate radial expansion of the inner polymeric tubular liner 24. For example, the inner polymeric tubular liner 24 can be provided with one or more longitudinal notches and/or cuts 36 extending along at least a portion of the length of the sheath 22. Such notches or cuts 36 can facilitate radial expansion of the inner polymeric tubular liner 24, thus accommodating passage of a delivery apparatus or other device. Such notches and/or cuts 36 can be provided near the inner surface 32, near the outer surface 37, and/or substantially through the entire thickness of the inner polymeric tubular liner 24. In aspects with a plurality of notches and/or cuts 36, such notches and/or cuts 36 can be positioned such that they are substantially equally spaced from one another circumferentially around the inner polymeric tubular liner 24. Alternatively, notches and cuts 36 can be spaced randomly in relation to one another or any other desired pattern. Some or all of any provided notches and/or cuts 36 can extend longitudinally along substantially the entire length of the sheath 22. Alternatively, some or all of any provided notches and/or cuts 36 can extend longitudinally only along a portion of the length of the sheath 22.

As shown in FIGS. 2B and 2C (which illustrates only the inner polymeric tubular liner 24), in some aspects, the inner polymeric tubular liner 24 contains at least one notch or cut 36 that extends longitudinally and parallel to an axis defined by the lumen 30, extending substantially the entire length of the sheath 22. Thus, upon introduction of a delivery apparatus, the inner polymeric tubular liner 24 can split open along the notch and/or cut 36 and expand, thus accommodating the delivery apparatus.

Additionally or alternatively, as shown in FIG. 2D, the outer polymeric tubular layer 26 can comprise one or more notches and/or cuts 36. Notches and/or cuts 36, in some aspects, do not extend through the entire thickness of the outer polymeric tubular layer 26. The notches and/or cuts 36 can be separable upon radial expansion of the sheath 22. The outer polymeric tubular layer 26 can be retractable longitudinally or able to be pulled back away from the intermediate tubular layer 28 and the inner polymeric tubular liner 24. In aspects with a retractable outer polymeric tubular layer 26, the outer polymeric tubular layer 26 can be retracted to accommodate or facilitate passage of a delivery apparatus through the lumen and then can be replaced to its original position on the sheath 22.

FIG. 3 illustrates an elevation view of the sheath 22 shown in FIG. 2A. In this view, only the outer polymeric tubular layer 26 is visible. The sheath 22 comprises a proximal end 38 and a distal end 40 opposite the proximal end 38. The sheath 22 can include a hemostasis valve inside the lumen of the sheath 22, at or near the proximal end 38 of the sheath 22.

Additionally, the sheath 22 can comprise a soft tip 42 at the distal end 40 of the sheath 22. Such a soft tip 42 can be provided with a lower hardness than the other portions of the sheath 22. In some aspects, the soft tip 42 can have a Shore hardness from about 25 D to about 40 D, including polymers having a Shore hardness of about 30 D or about 35 D.

As shown in FIG. 3, the unexpanded original outer diameter of the sheath 22 can be substantially constant across the length of the sheath 22, substantially from the proximal end 38 to the distal end 40. In alternative aspects, such as the ones illustrated in FIGS. 4A-4B, the original unexpanded outer diameter of the sheath 22 can decrease from the proximal end 38 to the distal end 40. As shown in the aspect in FIG. 4A, the original unexpanded outer diameter can decrease along a gradient from the proximal end 38 to the distal end 40. In alternative aspects, such as the one shown in FIG. 4B, the original unexpanded outer diameter of sheath 22, can incrementally step down along the length of the sheath 22, wherein the largest original unexpanded outer diameter is near the proximal end 38, and the smallest original unexpanded outer diameter is near the distal end 40 of the sheath 22.

As shown in FIGS. 5-6, the sheath 22 can be designed to locally expand as the prosthetic device is passed through the lumen of the sheath 22 and then substantially return to its original shape once the prosthetic device has passed through that portion of the sheath 22. For example, FIG. 5 illustrates a sheath 22 having a localized bulge 44, representative of a device being passed through the internal lumen of the sheath 22. FIG. 5 shows the device close to the proximal end 38 of the sheath 22, close to the area where the device is introduced into the sheath 22. FIG. 6 shows the sheath 22 of FIG. 5, with the device having progressed further along the sheath 22. The localized bulge 44 is now closer to the distal end 40 of the sheath 22, and thus, is about to be introduced to a patient's vessel. As evident from FIGS. 5 and 6, once the localized bulge associated with the device has passed through a portion of the lumen of the sheath 22, that portion of the sheath 22 can automatically return to its original shape and size, at least in part due to the materials and structure of the sheath b.

The sheath 22 has an unexpanded inner diameter equal to the inner diameter of the inner polymeric tubular liner (not visible in FIGS. 5-6) and an unexpanded outer diameter 46 equal to the outer diameter of the outer polymeric tubular layer 26. The sheath 22 is designed to be expanded to an expanded inner diameter and an expanded outer diameter 48, which are larger than the unexpanded inner diameter and the unexpanded outer diameter 46, respectively. In one representative aspect, the unexpanded inner diameter is about 16 Fr, and the unexpanded outer diameter 46 is about 19 Fr, while the expanded inner diameter is about 26 Fr, and the expanded outer diameter 48 is about 29 Fr. Different sheaths 22 can be provided with different expanded and unexpanded inner and outer diameters, depending on the size requirements of the delivery apparatus for various applications. Additionally, some aspects can provide more or less expansion depending on the particular design parameters, the materials, and/or configurations used.

In some aspects of a sheath according to the present disclosure, and as shown in section in FIG. 7 and in elevation in FIG. 8, the sheath 22 comprises an outer polymeric covering (or a second outer layer) 50, formed by the elongated tube as disclosed herein. In such aspects, the elongated tube can be positioned such that at least a portion of the inner surface of the elongated tube overlies at least a portion of the outer surface 52 of the first outer polymeric tubular layer 26. In such aspects, where the outer polymeric covering 50 is present, and it forms the outer covering (or the most outer layer), the outer polymeric tubular layer 26 can also be referred to as a first outer polymeric tubular layer 26, while the elongated tube can be referred to as a second. The outer polymeric covering 50 can provide a protective covering for the underlaying sheath 22. In some aspects, the outer polymeric covering 50 can contain a self-expandable sheath in a crimped or constrained state and then release the self-expandable sheath upon removal of the outer polymeric covering 50.

For example, in some aspects of a self-expandable sheath, the intermediate tubular layer 28 can comprise Nitinol and/or other shape memory alloys, and the intermediate tubular layer 28 can be crimped or radially compressed to a reduced diameter within the outer polymeric tubular layer 26 and the outer polymeric covering 50. Once the self-expandable sheath is at least partially inserted into a patient's vessel, the outer polymeric covering 50 can be slid back, peeled away, or otherwise at least partially removed from the sheath. To facilitate removal of the outer polymeric covering 50, a portion of the outer polymeric covering 50 can remain outside the patient's vessel, and that portion can be pulled back or removed from the sheath to allow the sheath to expand. In some aspects, substantially, the entire outer polymeric covering (the elongated tube as disclosed herein) 50 can be inserted, along with the sheath, into a patient's vessel. In these aspects, an external mechanism attached to the outer polymeric covering 50 can be provided, such that the outer polymeric covering can be at least partially removed from the sheath once the sheath is inserted into a patient's vessel.

In some aspects, once no longer constrained by the outer polymeric covering 50, the radially compressed intermediate tubular layer 28 can self-expand, causing expansion of the sheath along the length of the intermediate tubular layer 28. In some aspects, portions of the sheath can radially collapse, at least partially returning to the original crimped state, as the sheath is being withdrawn from the vessel after completion of the surgical procedure. In some aspects, such collapse can be facilitated and/or encouraged by an additional device or layer that, in some aspects, can be mounted onto a portion of the sheath prior to the sheath's insertion into the vessel.

The outer polymeric covering 50, in some aspects, is not adhered to the other layers of the sheath 22. For example, the outer polymeric covering 50 may be slidable with respect to the underlaying sheath, such that it can be easily removed or retracted from its initial position on the sheath 22.

Suitable materials for the outer polymeric covering 50 are disclosed in detail above.

Turning now to the intermediate tubular layer 28, several different configurations are possible. The intermediate tubular layer 28 is generally a thin, hollow, substantially cylindrical tube comprising an arrangement, pattern, structure, or configuration of wires or struts, however, other geometries can also be used. In some aspects, the intermediate tubular layer 28 is a braid. The intermediate tubular layer 28 can extend along substantially the entire length of the sheath 22, or alternatively, can extend only along a portion of the length of sheath 22. Suitable wires can be round, ranging from about 0.0005 inches thick to about 0.10 inches thick, or flat, ranging from about 0.0005 inches×0.003 inches to about 0.003 inches×0.007 inches. However, other geometries and sizes are also suitable for certain aspects. If a braided wire is used, the braid density can be varied. Some aspects have a braid density of from about thirty picks per inch to about eighty picks per inch and can include up to thirty-two wires in various braid patterns.

One representative aspect of an intermediate tubular layer comprises a braided Nitinol composite, which is at least partially encapsulated by an inner polymeric tubular member and an outer polymeric tubular member disposed on the inner and outer surfaces of the intermediate tubular layer, respectively. Such encapsulation by polymeric layers can be accomplished by, for example, fusing the polymeric layers to the intermediate tubular layer or dip coating the intermediate tubular layer. In some aspects, an inner polymeric tubular member, an intermediate tubular layer, and an outer polymeric tubular layer can be arranged on a mandrel, and the layers can then be thermally fused or melted into one another by placing the assembly in an oven or otherwise heating it. The mandrel can then be removed from the resulting sheath. In other aspects, dip coating can be used to apply an inner polymeric tubular member to the surface of a mandrel. The intermediate tubular layer can then be applied, and the inner polymeric tubular member is allowed to cure. The assembly can then be dip coated again, such as to apply a thin coating of, for example, polyurethane, which will become the outer polymeric tubular member of the sheath. The sheath can then be removed from the mandrel.

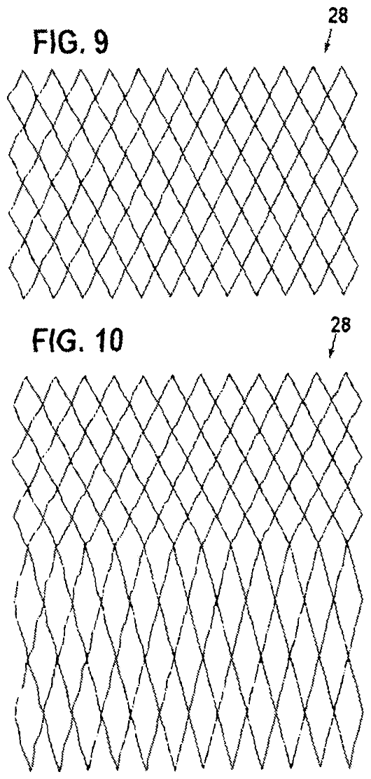

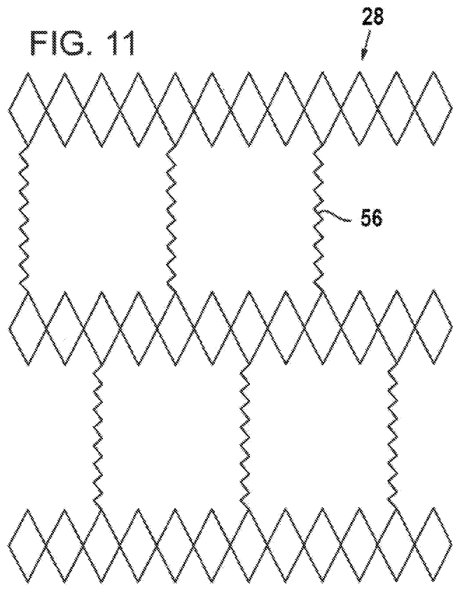

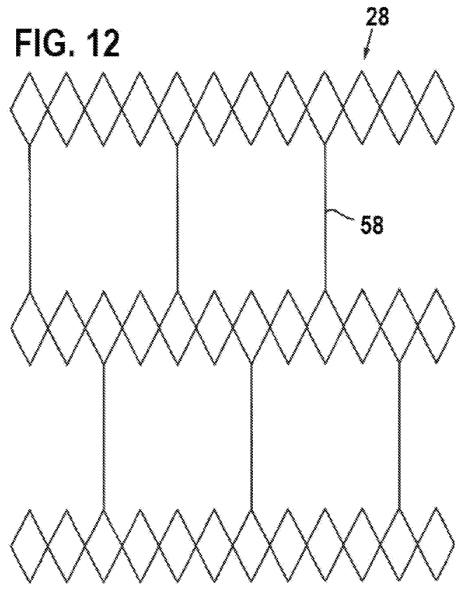

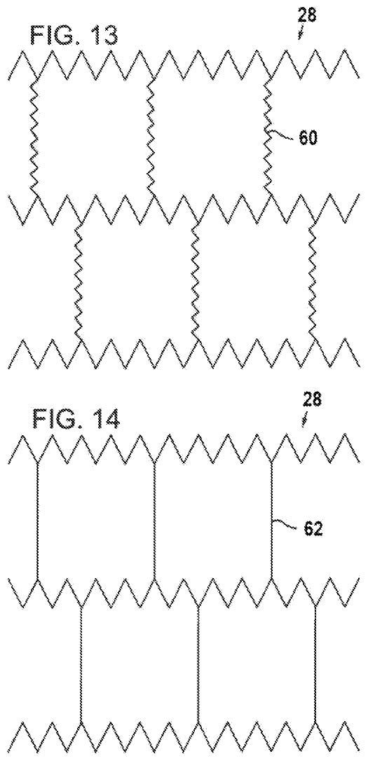

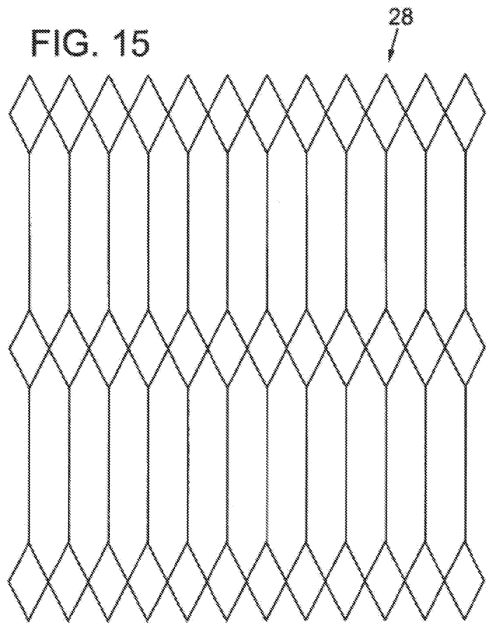

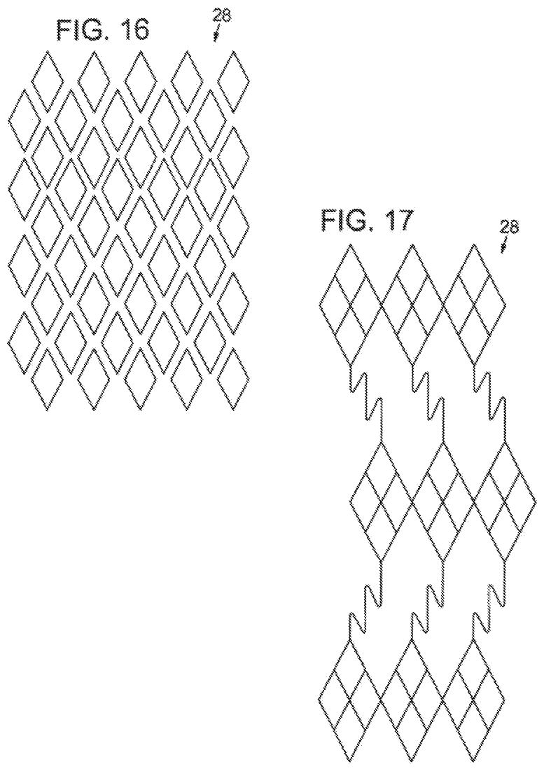

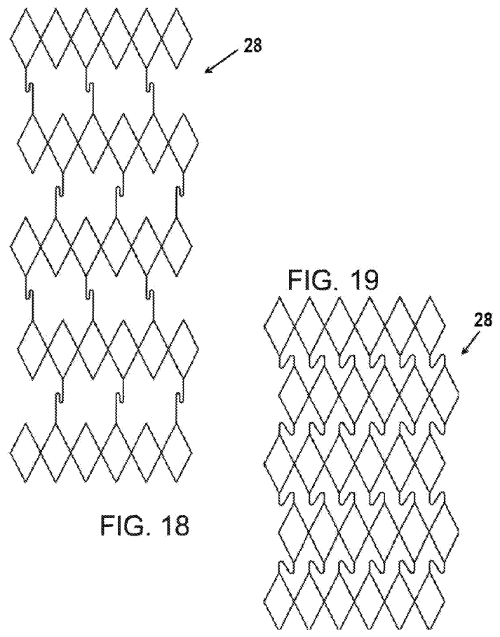

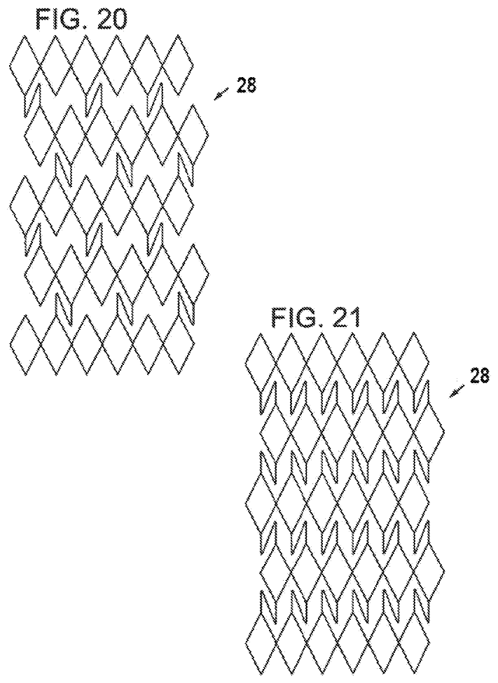

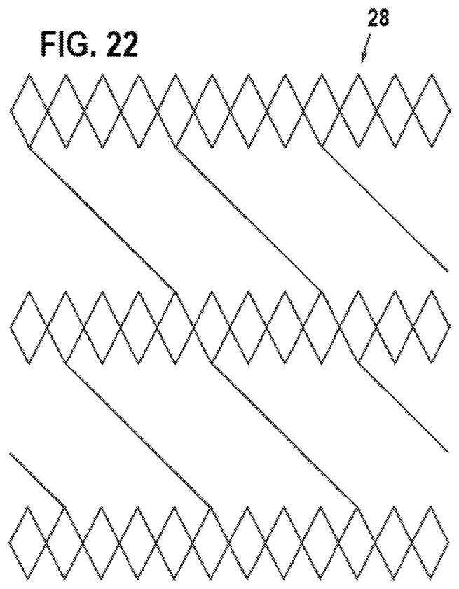

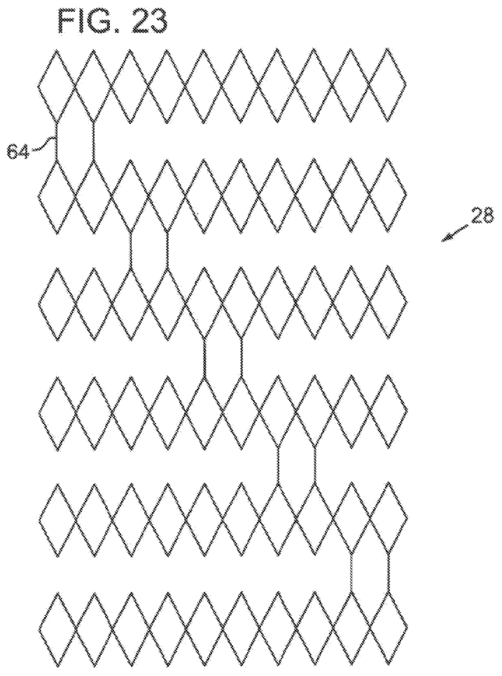

Additionally, the intermediate tubular layer 28 can be, for example, braided or laser cut to form a pattern or structure, such that the intermediate tubular layer 28 is amenable to radial expansion. FIGS. 9-23 illustrate partial elevation views of various structures for the intermediate tubular layer. Some illustrated structures, such as those shown in FIGS. 11-14 and 23, including at least one discontinuity. For example, the struts 56, 58, 60, 62, 64, shown in FIGS. 11, 12, 13, 14, and 23, respectively, resulting in a discontinuous intermediate tubular layer 28 in that the struts 56, 58, 60, 62, 64 separate adjacent sections of the intermediate tubular layer 28 from each other, where the sections are spaced apart from each other along a longitudinal axis parallel to the lumen of the sheath. Thus, the structure of the intermediate tubular layer 28 can vary from section to section, changing along the length of the sheath.

The structures shown in FIGS. 9-23 are not necessarily drawn to scale. Components and elements of the structures can be used alone or in combination within a single intermediate tubular layer 28. The scope of the intermediate tubular layer 28 is not meant to be limited to these particular structures; they are merely exemplary aspects.

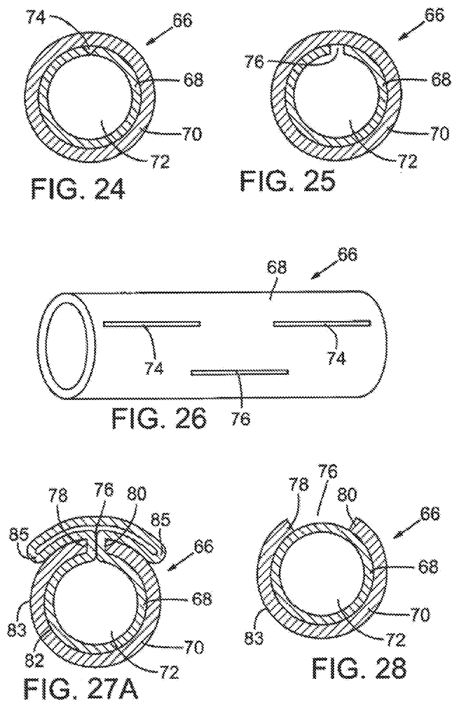

Alternative aspects of a sheath for introducing a prosthetic device are also described. For example, FIGS. 24-26 illustrate a section view and a perspective view, respectively, of a sheath 66 for introducing a prosthetic device into a body. The sheath 66 comprises an inner layer, such as inner polymeric liner 68, an outer layer, such as outer polymeric tubular layer 70, and a hemostasis valve (not shown). The inner polymeric liner 68 and the outer polymeric tubular layer 70 at least partially enclose a lumen 72, through which a delivery apparatus and prosthetic device can pass from outside the patient's body into the patient's vessel. Either or both of the inner polymeric liner 68 and the outer polymeric layer 70 can be provided with at least one longitudinal notch and/or cut to facilitate radial expansion of the sheath.

For example, FIG. 24 illustrates a longitudinal notch 74 in the inner polymeric liner 68 that can facilitate radial expansion of the sheath 66. The longitudinal notch 74 can separate or split open completely upon application of a radial force due to the insertion of a delivery apparatus or prosthetic device. Similarly, FIG. 25 illustrates a longitudinal cut 76 in the inner polymeric liner 68 that can also facilitate radial expansion of the sheath 66. The outer polymeric layer 70 can, additionally or alternatively, comprise one or more longitudinal cuts 76 or notches 74. Such cuts and/or notches, whether in the inner polymeric liner 68 or the outer polymeric layer 70, can extend substantially through the entire thickness of the layer or can extend only partially through the thickness of the layer. The cuts and/or notches can be positioned at or near the inner or outer surface, or both surfaces, of the inner and/or outer polymeric layers 68, 70.

FIG. 26 illustrates a perspective view of one aspect of an inner polymeric liner 68 with longitudinal notches 74 and a longitudinal cut 76. More or fewer notches 74 and/or cuts 76 can be provided. For clarity, the outer polymeric layer 70 is not shown in FIG. 26. As shown in FIG. 26, longitudinal notches 74 and/or cuts 76 can extend only along a portion of the length of sheath 66. In alternative aspects, one or more notches 74 and/or cuts 76 can extend substantially along the entire length of the sheath 66. Additionally, notches 74 and/or cuts 76 can be positioned randomly or patterned.

One particular aspect of a sheath 66 comprises a sheath having a notch or cut in the outer polymeric layer 70 or the inner polymeric layer (liner) 68 that extends longitudinally along approximately 75% of the length of the sheath 66. If such a notch or cut extends only partially through the associated layer, it can have a relatively low tear force, such as a tear force of about 0.5 lbs., so that the notch splits open relatively easily during use.

The inner polymeric liner 68 and the outer polymeric layer 70 can optionally be adhered together or otherwise physically associated with one another. The amount of adhesion between the inner polymeric liner 68 and the outer polymeric layer 70 can be variable over the surfaces of the layers. For example, little to no adhesion can be present at areas around or near any notches and/or cuts present in the layers so as not to hinder radial expansion of the sheath 66. Adhesion between the layers can be created by, for example, thermal bonding and/or coatings. Aspects of a sheath 66 can be formed from an extruded tube, which can serve as the inner polymeric layer (liner) 68. The inner polymeric layer (liner) 68 can be surface-treated, such as by plasma etching, chemical etching, or other suitable methods of surface treatment. By treating the surface of the inner polymeric liner 68, the outer surface of the inner polymeric liner 68 can have areas with altered surface angles that can provide better adhesion between the inner polymeric liner 68 and the outer polymeric layer 70. The treated inner polymeric liner can be dip coated in, for example, a polyurethane solution to form the outer polymeric layer 70. In some configurations, the polyurethane may not adhere well to untreated surface areas of the inner polymeric liner 68. Thus, by surface treating only surface areas of the inner polymeric liner 68 that are spaced away from the areas of expansion (e.g., the portion of the inner polymeric liner 68 near notches 74 and/or cuts 76), the outer polymeric layer 70 can be adhered to some areas of the inner polymeric liner 68, while other areas of the inner polymeric liner 68 remain free to slide relative to the outer polymeric layer 70, thus allowing for expansion of the diameter of the sheath 66. Thus, areas around or near any notches 74 and/or cuts 76 can experience little to no adhesion between the layers, while other areas of the inner and outer polymeric layers 68, 70 can be adhesively secured or otherwise physically associated with each other.

As with previously disclosed aspects, the aspects illustrated in FIGS. 24-26 can be applied to sheaths having a wide variety of inner and outer diameters. Applications can utilize a sheath of the present disclosure with an inner diameter of the inner polymeric liner 68 that is expandable to an expanded diameter of from about 3 Fr to about 26 Fr, including exemplary values of about 5 Fr, about 10 Fr, about 15 Fr, about 20 Fr, and about 25 Fr. The expanded diameter can vary slightly along the length of the sheath 66. For example, the expanded outer diameter at the proximal end of the sheath 66 can range from about 3 Fr to about 28 Fr, including exemplary values of about 5 Fr, about 10 Fr, about 15 Fr, about 20 Fr, and about 25 Fr, while the expanded outer diameter at the distal end of the sheath 66 can range from about 3 Fr to about 25 Fr, including exemplary values of about 5 Fr, about 10 Fr, about 15 Fr, about 20 Fr, and about 25 Fr. Aspects of a sheath 66 can expand to an expanded outer diameter that is from about 10% greater than the original unexpanded outer diameter to about 100% greater than the original unexpanded outer diameter.

In some aspects, the outer diameter of the sheath 66 gradually decreases from the proximal end of the sheath 66 to the distal end of the sheath 66. For example, in one aspect, the outer diameter can gradually decrease from about 26 Fr at the proximal end to about 18 Fr at the distal end. The diameter of the sheath 66 can transition gradually across substantially the entire length of the sheath 66. In other aspects, the transition or reduction of the diameter of the sheath 66 can occur only along a portion of the length of the sheath 66. For example, the transition can occur along a length from the proximal end to the distal end, where the length can range from about 0.5 inches to about the entire length of the sheath 66.

Suitable materials for the inner polymeric liner 68 can have high elastic strength and include materials discussed in connection with other aspects, especially Teflon (PTFE), polyethylene (e.g., high density polyethylene), fluoropolymers, or combinations thereof. In some aspects, the inner polymeric liner 68 preferably has a low coefficient of friction, such as a coefficient of friction of from about 0.01 to about 0.5, including exemplary values of about 0.05, about 0.1, about 0.15, about 0.2, about 0.25, about 0.3, about 0.35, about 0.4, and about 0.45. In yet some other aspects of a sheath, 66 can comprise an inner polymeric liner 68 having a coefficient of friction of about 0.1 or less, or 0.05 or less.

Likewise, suitable materials for the outer polymeric layer 70 include materials discussed in connection with other aspects and other thermoplastic elastomers and/or highly elastic materials.

The Shore hardness of the outer polymeric layer 70 can be varied for different applications and aspects. Some aspects include an outer polymeric layer with a Shore hardness of from about 25 A to about 80 A, including exemplary values of about 30 A, about 35 A, about 40 A, about 45 A, about 50 A, about 55 A, about 60 A, about 65 A, about 70 A, and about 80 A. Yet, in other aspects, an outer polymeric layer can have a Shore hardness of from about 20 D to about 40 D, including exemplary values of about 25 D, about 30 D, and about 35 D. One aspect comprises a readily available polyurethane with a Shore hardness of 72 A. Another particular aspect comprises a polyethylene inner polymeric layer dipped in polyurethane or silicone to create the outer polymeric layer.

The sheath 66 can also include a radiopaque filler or marker, as described above. In some aspects, a distinct radiopaque marker or band can be applied to some portion of the sheath 66. For example, a radiopaque marker can be coupled to the inner polymeric liner 68, the outer polymeric layer 70, and/or can be positioned in between the inner and outer polymeric layers 68, 70.

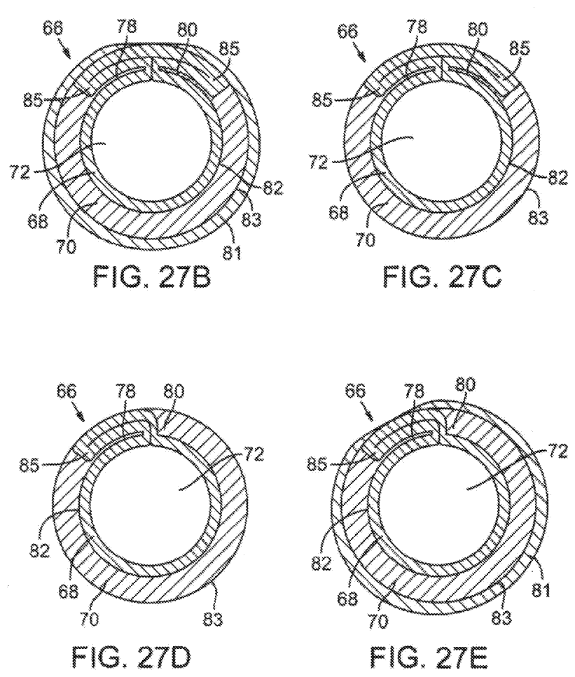

FIGS. 27A-27E and 28 illustrate section views of various aspects of unexpanded (FIGS. 27A-27E) and expanded (FIG. 28) sheaths 66 according to the present disclosure. The sheath 66 includes a split outer polymeric tubular layer 70 having a longitudinal cut 76 through the thickness of the outer polymeric tubular layer 70 such that the outer polymeric tubular layer 70 comprises a first portion 78 and a second portion 80 separable from one another along the cut 76. An expandable inner polymeric liner 68 is associated with an inner surface 82 of the outer polymeric tubular layer 70, and, in the unexpanded configuration shown in FIG. 27A, a portion of the inner polymeric liner 68 extends through a gap created by the cut 76 and can be compressed between the first and second portions 78, 80 of the outer polymeric tubular layer 70. Upon expansion of the sheath 66, as shown in FIG. 28, first and second portions 78, 80 of the outer polymeric tubular layer 70 have separated from one another, and the inner polymeric liner 68 is expanded to a substantially cylindrical tube. In some aspects, two or more longitudinal cuts 76 may be provided through the thickness of the outer polymeric tubular layer 70. In such aspects, a portion of the inner polymeric liner 68 may extend through each of the longitudinal cuts 76 provided in the outer polymeric tubular layer 70.

In certain aspects, the inner polymeric liner 68 comprises one or more materials that are elastic and amenable to folding and/or pleating. For example, FIG. 27A illustrates an inner polymeric liner 68 with folded regions 85. As seen in FIGS. 27A-27E, the sheath 66 can be provided with one or more folded regions 85. Such folded regions 85 can be provided along a radial direction and substantially conform to the circumference of the outer polymeric tubular layer 70. At least a portion of the folded regions 85 can be positioned adjacent the outer surface 83 of the outer polymeric tubular layer 70.

Additionally, as shown in FIGS. 27B and 27E, at least a portion of the folded region or regions 85 can be overlapped by an outer covering, such as outer polymeric covering 81. In such aspects, the outer polymeric covering 81 can be the elongated tube as disclosed herein. The outer polymeric covering 81 can be adjacent at least a portion of the outer surface 83 of the outer polymeric tubular layer 70. The outer polymeric covering 81 serves to at least partially contain the folded regions 85 of the inner polymeric liner 68 and can also prevent the folded regions 85 from separating from the outer polymeric tubular layer 70 when, for example, the sheath 66 undergoes bending. In some aspects, the outer polymeric covering (or elongated tube as described herein) 81 can be at least partially adhered to the outer surface 83 of the outer polymeric tubular layer 70. The outer polymeric covering (elongated tube) 81 in certain aspects can increase the stiffness and/or durability of the sheath 66, while in other aspects and, as also disclosed herein, it can reduce the push force required to push a medical device through the sheath. In certain aspects, however, and as shown in FIGS. 27B and 27E, the outer polymeric covering 81 may not entirely overlap the circumference of the sheath 66. For example, the outer polymeric covering 81 may be provided with first and second ends, where the ends do not contact one another. In these aspects, only a portion of the folded region 85 of the inner polymeric liner 68 is overlapped by the outer polymeric covering 81.

In aspects having a plurality of folded regions 85, the regions can be equally displaced from each other around the circumference of the outer polymeric tubular layer 70. Alternatively, the folded regions can be off-center, different sizes, and/or randomly spaced apart from each other. While portions of the inner polymeric liner 68 and the outer polymeric tubular layer 70 can be adhered or otherwise coupled to one another, the folded regions 85 preferably are not adhered or coupled to the outer polymeric tubular layer 70. For example, adhesion between the inner polymeric liner 68 and the outer polymeric tubular layer 70 can be highest in areas of minimal expansion.

One particular aspect of the sheath, as illustrated in FIGS. 27A-28, comprises a polyethylene (e.g., high density polyethylene) outer polymeric tubular layer 70 and a PTFE inner polymeric liner 68. However, other materials are suitable for each layer, as described above. Generally, suitable materials for use with the outer polymeric tubular layer 70 include materials having a high stiffness or modulus of strength that can support the expansion and contraction of the inner polymeric liner 68.

In certain aspects, where the inner polymeric liner 68 comprises one or more folded portions, and as disclosed above, the inner liner can be selectively etched. In such aspects, at least a portion of the outer surface of the at least one folded portion of the inner liner is not etched along at least a portion of the sheath length. In still further aspects, the outer surface of the inner liner comprises one or more nonetched portions along the sheath length. Again, in the aspects where are a plurality of nonetched portions, such portions can be at any location on the sheath. In certain aspects, for example, and without limitation, each of the one or more nonetched portions is followed by an etched portion. While in other aspects, the one or more nonetched portions comprise the outer surface of the at least one folded portion.

In certain aspects, the sheaths disclosed herein can exhibit an insertion force of less than about 60 N, less than about 50 N, less than about 40 N, or less than about 30 N. In still further aspects, the sheath can exhibit a reduction in an insertion force of at least about 10%, at least about 20%, at least about 30%, at least about 40%, at least about 50%, at least about 60%, at least about 70%, at least about 80%, at least about 90%, or at least about 100%, when compared to a substantially identical reference sheath that does not comprise the first compound composition and the selectively etched inner liner

In still further aspects, where the sheath comprises an inner liner that is selectively etched and has nonetched portions as disclosed above, such a sheath can exhibit even a substantial reduction in the insertion force when compared to a substantially identical sheath that does not comprise similar nonetched portions.

In some aspects, the outer polymeric tubular layer 70 comprises the same material or combination of materials along the entire length of the outer polymeric tubular layer 70. In alternative aspects, the material composition can change along the length of the outer polymeric tubular layer 70. For example, the outer polymeric tubular layer can be provided with one or more segments, where the composition changes from segment to segment. In one particular aspect, the Durometer rating of the composition changes along the length of the outer polymeric tubular layer 70 such that segments near the proximal end comprise a stiffer material or combination of materials, while segments near the distal end comprise a softer material or combination of materials. This can allow for a sheath 66 having a relatively stiff proximal end at the point of introducing a delivery apparatus while still having a relatively soft distal tip at the point of entry into the patient's vessel.

As with other disclosed aspects, the aspects of sheath 66 shown in FIGS. 27A-28 can be provided in a wide range of sizes and dimensions. For example, the sheath 66 can be provided with an unexpanded inner diameter of from about 3 Fr to about 26 Fr, including exemplary values of about 5 Fr, about 10 Fr, about 15 Fr, about 20 Fr, and about 25 Fr. In some aspects, the sheath 66 has an unexpanded inner diameter of from about 15 Fr to about 16 Fr. In some aspects, the unexpanded inner diameter of the sheath 66 can range from about 3 Fr to about 26 Fr, including exemplary values of about 5 Fr, about 10 Fr, about 15 Fr, about 20 Fr, and about 25 Fr at or near the distal end of sheath 66, while the unexpanded inner diameter of the sheath 66 can range from about 3 Fr to about 28 Fr, including exemplary values of about 5 Fr, about 10 Fr, about 15 Fr, about 20 Fr, and about 25 Fr at or near the proximal end of sheath 66. For example, in one unexpanded aspect, the sheath 66 can transition from an unexpanded inner diameter of about 16 Fr at or near the distal end of the sheath 66 to an unexpanded inner diameter of about 26 Fr at or near the proximal end of the sheath 66.

The sheath 66 can be provided with an unexpanded outer diameter of from about 3 Fr to about 30 Fr, including exemplary values of about 5 Fr, about 10 Fr, about 15 Fr, about 20 Fr, and about 25 Fr and, in some aspects has an unexpanded outer diameter of from about 18 Fr to about 19 Fr. In some aspects, the unexpanded outer diameter of the sheath 66 can range from about 3 Fr to about 28 Fr, including exemplary values of about 5 Fr, about 10 Fr, about 15 Fr, about 20 Fr, and about 25 Fr at or near the distal end of sheath 66, while the unexpanded outer diameter of the sheath 66 can range from about 3 Fr to about 30 Fr, including exemplary values of about 5 Fr, about 10 Fr, about 15 Fr, about 20 Fr, and about 25 Fr at or near the proximal end of sheath 66. For example, in one unexpanded aspect, the sheath 66 can transition from an unexpanded outer diameter of about 18 Fr at or near the distal end of the sheath 66 to an unexpanded outer diameter of about 28 Fr at or near the proximal end of the sheath 66.

The thickness of the inner polymeric liner 68 can vary, but in some preferred aspects is from about 0.002 inches to about 0.015 inches, including exemplary values of about 0.003 inches, about 0.004 inches, about 0.005 inches, about 0.006 inches, about 0.007 inches, about 0.008 inches, about 0.009 inches, about 0.010 inches, about 0.011 inches, about 0.012 inches, about 0.013 inches, and about 0.014 inches. In some aspects, expansion of the sheath 66 can result in expansion of the unexpanded outer diameter of from about 10% or less to about 430% or more, for example, and without limitation about 1%, about 5%, about 10%, about 20%, about 50%, about 70%, about 100%, about 120%, about 150%, about 200%, about 250%, about 300%, about 350%, about 400%, about 450%, about 500%, or even more.

As with other illustrated and described aspects, the aspects shown in FIGS. 27A-28 can be provided with a radiopaque filler and/or a radiopaque tip marker, as described above. The sheath 66 can be provided with a radiopaque tip marker provided at or near the distal tip of the sheath 66. Such a radiopaque tip marker can comprise materials such as those suitable for the radiopaque filler, platinum, iridium, platinum/iridium alloys, stainless steel, other biocompatible metals, or combinations thereof.

In still further aspects, where the folded portions can be present, the at least one folded portion can comprise a first folded edge and a second folded edge and an overlapping portion extending circumferentially between the first and second folded edges, the overlapping portion comprising an overlap in a radial direction of at least two thicknesses of the inner liner, and wherein the first folded edge is configured to move closer to the second folded edge to shorten the overlapping portion at a local axial location during application of a radial outward force by passage of the medical device and wherein shortening of the overlapping portion corresponds with a local expansion of the lumen. Yet in other aspects, the at least one folded portion comprises a first folded edge and a second folded edge and an overlapping portion extending circumferentially between the first and second folded edges, the overlapping portion comprising an overlap in a radial direction of at least two thicknesses of the inner liner, wherein the first folded edge is configured to move closer to the second folded edge to shorten the overlapping portion at a local axial location during application of a radial outward force by passage of the medical device and wherein shortening of the overlapping portion corresponds with a local expansion of the lumen, and wherein the outer layer includes a first longitudinally extending edge and a second longitudinally extending edge configured to separate as the sheath expands, the first longitudinal extending edge and an overlapping portion of the outer layer extending over the second longitudinally extending edge when the sheath is not expanded.

In still further aspects, it is understood that the inner liner can be configured to expand to a substantially cylindrical tube.

In still further aspects, the sheath disclosed herein and comprising the elongated tube, as described herein, can further comprise an inner tubular layer comprising a longitudinal slit and partially defining an inner lumen, a first outer tubular layer enveloping the inner layer, the outer tubular layer comprising a longitudinally extending, folded flap that overlies a portion of an outer surface of the outer layer when the sheath is in an unexpanded state, and the elongated tube positioned such that the inner surface of the elongated tube overlies at least a portion of the outer surface of the first outer tubular layer.

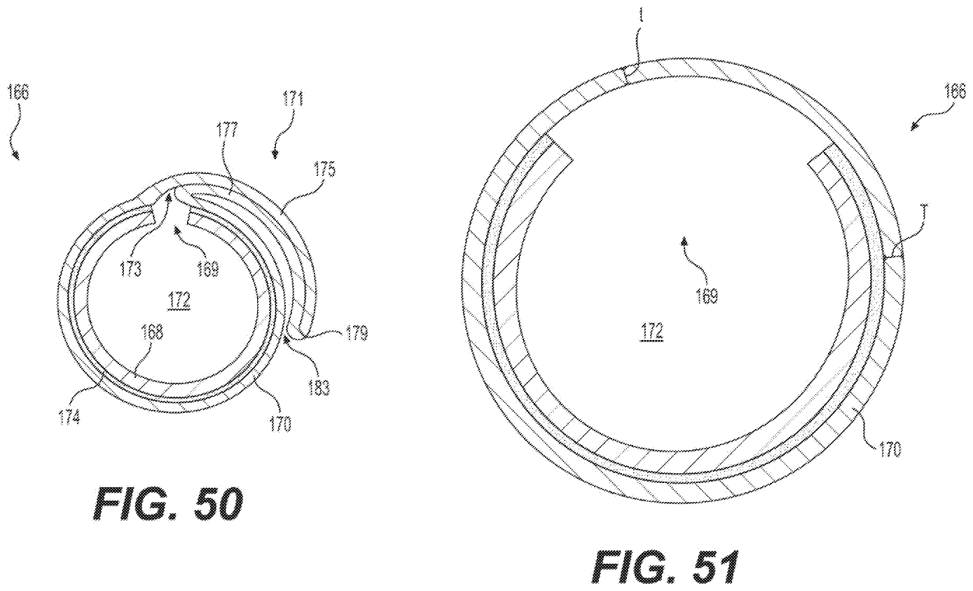

Such exemplary aspects are described in detail below. FIGS. 50 and 51 show cross-sectional views of an expandable sheath 166 having an inner tubular layer 168 with a longitudinal slit 169. In some aspects, the longitudinal slit 169 extends the entire length of the inner tubular layer 168. A first outer tubular layer 170 envelops the inner tubular layer 168 and includes a longitudinally extending, folded flap 171. In some aspects, the folded flap 171 extends the entire length of the first outer tubular layer 170. The folded flap 171 overlies a portion of the outer surface 183 of the first outer tubular layer 170 when the sheath is in an unexpanded state (FIG. 50). When a prosthetic device is moved through an inner lumen 172 of the sheath 166, it applies an outwardly directed radial force on the inner tubular layer 168 that widens the longitudinal slit 169 and unfolds the folded flap 171. FIG. 51 shows the sheath 166 in the unexpanded state, with the longitudinal slit 169 widened, and the first outer tubular layer 170 unfolded.

The folded flap 171 of the first outer tubular layer 170 has a base 173. The base 173 can be positioned radially outwardly from the longitudinal slit 169. In some aspects, the base 173 is centered over the longitudinal slit 169. The folded flap 171 further includes a longitudinally extending overlying portion 175 extending from the base 173 to a longitudinally extending crease 179 at the edge of the flap 171. The longitudinally extending overlying portion 175 overlies a longitudinally extending underlaying portion 177 and is separated from the underlaying portion 177 by the crease 179. Underlaying portion 177 contacts the outer surface 183 of the first outer tubular layer 170 when the sheath is in an unexpanded state, as shown in FIG. 50.

Some aspects of sheath 166 can include multiple longitudinally extending folded flaps that overlie portions of the outer surface 183 of the first outer tubular layer 170, positioned at various locations around the circumference of sheath 166. For example, 2, 3, 4, 5, 6, 7, 8, 9, or 10 longitudinally extending folded flaps can be positioned around the circumference. In some aspects, these multiple folded flaps are equally spaced around the circumference of the sheath 166.

The folded flap 171 extends circumferentially around a portion of the sheath 166. In some aspects, the longitudinally extending flap 171 extends around about 20% to about 40% of the outer circumference of the first outer tubular layer 170 when the sheath 166 is unexpanded (including about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, and about 40% of the outer circumference of the first outer tubular layer 170). In one example, for a sheath having a 14 F (4.7 millimeters) unexpanded outer diameter, the folded flap extends around about 85 to about 120 degrees (including exemplary values of about 90, about 95, about 100, about 105, about 110, and 115 degrees), or about 23%-35%, (including exemplary values of about 24%, about 25%, about 26%, about 27%, about 28%, about 29%, about 30%, about 31%, about 32%, about 33%, and about 34%) of the outer circumference (resulting in an inner lumen having an expanded diameter of about 7.6 millimeters to about 8.4 millimeters, including exemplary values of about 7.7 mm, about 7.8 nm, about 7.9 mm, about 8.0 mm, about 8.1 mm, about 8.2 mm, and about 8.3 mm, which can be used with a valve having a 6.4 millimeter crimped outer diameter, and a 26 millimeter expanded outer diameter).

In FIG. 51, the wall thickness of flap 171 is labeled t, and the wall thickness of other parts of the first outer tubular layer 170 are labeled T. In some aspects, a portion of the flap 171 (such as, for example, the underlaying portion 177 or the overlying portion 175) can have a wall thickness t that is thinner than a wall thickness (T) of other portions of the outer tubular layer. This variation in wall thickness promotes even column strength around the circumference of the sheath 166, which reduces kinking and minimizes the total outer diameter of the sheath. The wall thickness variation can also facilitate the folding process. In some aspects, the entire flap 171 has a wall thickness t that is thinner than a wall thickness T of the remainder of the outer tubular layer. In one example, the wall thickness of t can be from about 0.003 inches to about 0.007 inches, including exemplary values of about 0.004 inches, about 0.005 inches, and about 0.006 inches, while the wall thickness of T can be from about 0.008 inches to about 0.012 inches, including exemplary values of about 0.009 inches and about 0.01 inches. In other aspects, such as the one shown in FIG. 51, the wall thickness t of flap 171 is about equal to the wall thickness T of the remainder of the first outer tubular layer 170.

The first outer tubular layer 170 is formed of a material having a low coefficient of friction, a high tensile modulus, and a high ultimate tensile strength in order to improve the push force transmission through the length of sheath 166 while reducing kinking. Good push force transmission means that the force applied by the practitioner to advance the sheath is predictable, responsive, and consistent along the length of the sheath. However, an excessively high tensile modulus may limit the ability of the longitudinally extending flap 171 to open, which could hamper push force transmission. A desirable range for the tensile modulus of the first outer tubular layer 170 is from about 300 MPa to about 2,000 MPa (including about 300 MPa, about 400 MPa, about 500 MPa, about 600 MPa, about 700 MPa, about 800 MPa, about 900 MPa, about 1000 MPa, about 1100 MPa, about 1200 MPa, about 1300 MPa, about 1400 MPa, about 1500 MPa, about 1600 MPa, about 1700 MPa, about 1800 MPa, about 1900 MPa, and about 2000 MPa). In some aspects, the tensile modulus may preferably be at least 700 MPa. High axial and radial stiffnesses enable the sheath to be easily inserted and to resist collapse within the body.

The ultimate tensile strength of the first outer tubular layer 170 can be at least 50 MPa. A high ultimate tensile strength helps to prevent the material from tearing while the prosthetic device is advancing through the sheath.

In some exemplary and unlimiting aspects, the first outer tubular layer 170 of the aspects shown in FIGS. 50 and 51 can comprise high density polyethylene (HDPE), polyamide, co-polyamide, polyether block amide (PEBAX®), or a blend of polyamide. Materials having shape memory properties are advantageous because the first outer tubular layer 170 can be given a bias toward the folded state (for example, by heat setting). This facilitates refolding of the first outer tubular layer 170 after passage of a prosthetic device. PEBAX® is an exemplary shape memory material that may be heat set toward the folded state.

In some aspects, the outer surface 183 of the first outer tubular layer 170 can include a hydrophilic coating. In some aspects, a bond can be created between the underlaying portion 177 of the folded flap 171 and the outer surface 183 of the first outer tubular layer 170. The bond can be a thermal bond (with portions of the contacting layers melted together), or it can be a separate layer of adhesive.

As discussed above, the inner tubular layer 168 of the aspects shown in FIGS. 50 and 51 include a longitudinal slit 169. The inner tubular layer 168 can comprise a first longitudinally extending end 178 and a second longitudinally extending end 180, the first and second longitudinally extending ends 178, 180 defining the longitudinal slit 169.

The inner tubular layer 168 forms a low friction barrier between a passing prosthetic device and the first higher friction outer tubular layer 170. The inner tubular layer 168 extends around at least 80% (or at least 85%, or at least 90%, or at least 95%) of the circumference of the inner lumen 172 when the sheath 166 is in an unexpanded state. This high degree of coverage limits contact between the passing prosthetic device and the first higher friction outer tubular layer 170. In some aspects, the coefficient of friction per ASTM D1894 of the inner tubular layer 168 (static or dynamic) is 0.30 or less, or 0.25 or less, or 0.20 or less, or 0.1 or less, or 0.05 or less. In other aspects, the coefficient of friction is 0.25 or less.

In some aspects, the low-friction inner tubular layer 168 comprises, or is formed of, a material having a tensile modulus of at least about 300 MPa (and up to about 1,400 MPa, including exemplary values of about 350 MPa, about 400 MPa, about 450 MPa, about 500 MPa, about 550 MPa, about 600 MPa, about 650 MPa, about 700 MPa, about 750 MPa, about 800 MPa, about 850 MPa, about 900 MPa, about 1,000 MPa, about 1,050 MPa, about 1,100 MPa, about 1,150 MPa, about 1,200 MPa, about 1,250 MPa, about 1,300 MPa, and about 1,350 MPa) to provide good push force transmission while providing kink resistance. This material could be, for example, high density polyethylene or a fluoropolymer. Exemplary fluoropolymers include polytetrafluoroethylene, ethylene fluorinated ethylene propylene, or perfluoro alkoxy.

A tie layer 174 can be positioned between the two layers, thereby adhering the inner tubular layer 168 to the first outer tubular layer 170. The tie layer 174 can be formed of polyurethane or functionalized polyolefin in some aspects. In some aspects, the contacting surface of the inner tubular layer 168 can be etched to improve bonding to the tie layer 174. For example, an inner tubular layer 168 that includes, or is formed of, a fluoropolymer might be etched on its outer surface to improve thermal bonding to the tie layer 174. In yet further aspects, the inner tubular layer 168 can be selectively etched as described above.

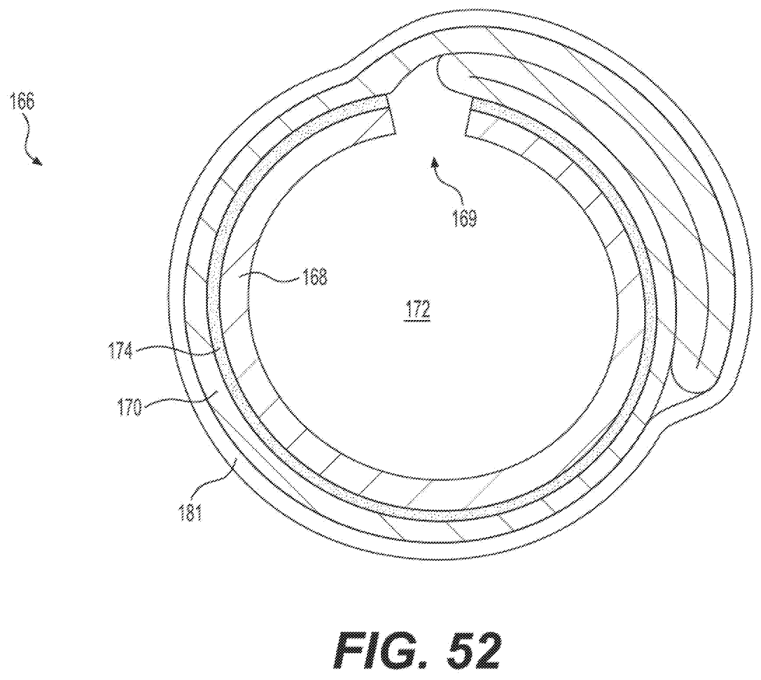

As shown in FIG. 52, the sheath comprises the disclosed in detail herein the elongated tube forming a second outer layer 181. In such aspects, the elongated tube forms a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer. It is understood that this second outer layer, in some aspects, is also referred to as an outer jacket. The outer jacket 181 is formed as disclosed herein and can comprise a first polymer layer, wherein the first polymer layer comprises a first compound composition comprising from greater than 0 wt % to less than 100 wt % of a polymer comprising a polyether block amide, a polyurethane, or a combination thereof; less than about 65 wt % of an inorganic filler based on a total weight of the first compound composition; and up to about 20 wt % of a solid lubricant filler based on a total weight of the first compound composition. The additional exemplary aspects of the composition and characteristics of such a second outer layer (outer jacket) are disclosed in detail above.

As mentioned above, in some aspects, the first outer tubular layer 170 can be included or can be formed of a shape memory material (for example, a heat-set polymer such as PEBAX®) that facilitates refolding of the first outer tubular layer 170 after expansion. In aspects disclosed herein, the second outer layer (outer jacket) 181 can extend over and envelop the first outer tubular layer 170. The refolding of the first outer tubular layer 170 can preferably return the sheath 166 back to its original outer diameter or to a value close to the original outer diameter (for example, to within about 10%, about 20%, about 30%, about 40%, or about 50% of the original outer diameter).

Methods of using the sheath of FIGS. 50-52 include first inserting an expandable sheath 166 into the vasculature of a subject and advancing the prosthetic device through an inner lumen 172 of the expandable sheath 166. The prosthetic device applies an outwardly directed radial force to the inner tubular layer 168 of the expandable sheath 166. In some aspects, the outwardly directed radial force is transmitted through the inner tubular layer 168, the tie layer 174, and the first outer tubular layer 170. The outwardly directed radial force widens a longitudinal slit 169 in the inner tubular layer 168. The widening of the longitudinal slit 169 travels the full length of the expandable sheath in some aspects.

The outwardly directed radial force further unfolds the longitudinally extending flap 171 of the first outer tubular layer 170 to expand the expandable sheath. The unfolding of the flap 171 can include sliding a longitudinally extending overlying portion 175 circumferentially against a longitudinally extending underlaying portion 177 of flap 171. The underlaying portion 177 can slide circumferentially against an outer surface 183 of the first outer tubular layer 170. In some aspects, the unfolding of the longitudinally extending flap 171 occurs at a position radially outward from the longitudinal slit 169 of the inner tubular layer 168. The unfolding of the flap 171 can extend the full length of the expandable sheath 166 in some aspects.

The longitudinal slit 169 of the inner tubular layer 168 narrows once the outwardly directed radial force has ceased (i.e., once the prosthetic device has passed by). The slit 169 may narrow back to its original width or to a value close to the original width (for example, to a value within 10% of the original width). The narrowing can occur along the entire length of the sheath 166. The prosthetic device is then delivered to the procedure site.

The longitudinally extending flap 171 at least partially refolds once the prosthetic device ceases to apply the outwardly directed radial force (i.e., once it has passed by). In some aspects, the longitudinally extending flap 171 refolds itself due to a shape memory bias toward the folded state. In some aspects, an inwardly directed radial force is applied to the outer surface 183 of the first outer tubular layer 170 to refold the longitudinally extending flap 171 (for example, by the second outer layer (outer jacket) 181).

An example method of making the sheath is as follows. These steps are not meant to be limiting. The steps given can be reordered as needed. Other steps can be added, or in other examples, some steps may not be necessary. Sizes are approximate. Disclosed herein is an exemplary aspect of making a sheath where the elongated tube or recoverable outer jacket covers the whole length of the expandable sheath shaft assembly. In certain aspects, the elongate tube and the expandable sheath shaft assembly can be about 380 mm long (15 inches). 1) Start with a PTFE inner layer of about 0.200 inch inner diameter (ID), wall thickness about 0.004 inches, 2) load PTFE inner layer on to a tapering mandrel from about 0.200 inches to about 0.187 inches, 3) stretch on to an 0.187 inch outer diameter (OD) mandrel section under heat, 4) flare proximal end of 0.200 inch ID PTFE to 0.340 inches ID under heat, 5) by expanding with air pressure, load tie layer such as Tecoflex 80A having about 0.200 inch ID and 0.004 inch wall thickness over the PTFE inner layer along the body section, 6) adhere tie layer to inner layer, for example, by covering it with FEP (fluorinated ethylene propylene) heat shrink tubing and applying heat, 7) remove FEP thermal shrink tubing if it was used, 8) create a longitudinal slit along the body section of the assembly, 9) load the subassembly having a slit on to a 0.187 inch OD mandrel, 10) load outer layer over the body, 11) fold outer layer, 12) heat set the fold, for example, by inserting the subassembly with a fold inside a heat shrink tubing and placing the assembly into the oven, 13) remove the shrink tubing if it was used, 14) add the second outer layer; and 14) remove sheath from the mandrel.

The outer jacket can be expanded to a larger diameter by applying air pressure to the inner diameter of the outer jacket. The assembled sheath can then be inserted into the expanded outer jacket for the desired length. Air pressure can then be released to allow the outer jacket to decrease in diameter to the original diameter.

Also disclosed an exemplary aspect of making a sheath where the elongated tube or recoverable outer jacket covers the proximal side of the expandable sheath shaft assembly. In such exemplary aspects, the sheath shaft having about 380 mm (15 inches) length can be assembled. For example, a tapered mandrel can be inserted into the sheath shaft assembly's inner diameter. The outer jacket or strain relief tubing, as disclosed here, can be cut to about 105 mm +/− 10 mm (4.15″ +/− 0.40″) and positioned on the sheath shaft assembly. The outer jacket can have an inner diameter big enough to slide the outer jacket over the sheath shaft assembly, especially with the outer jacket comprised of the low friction PTFE incorporating layer. An FEP (fluoroethylene propylene) heat shrink tubing can then be flared on the proximal end. Flared FEP heat shrink tubing can be slid over the outer jacket. Heat can be used to fuse the outer jacket to the sheath shaft assembly. The distal end of the outer jacket can be heated to adhere to the distal end of the outer jacket to the sheath shaft assembly. FEP heat shrink tubing can then be removed.

Further, the mandrel can be removed. Still, further, the proximal end of the sheath and the outer jacket assembly can beflared over a heated flaring tool. It is understood that in some exemplary and unlimiting aspects, other manufacturing steps can be present such as the flushing tube and the housing bonding, hydrophilic coating, inspection, the valves, and the sleeve assembly, and leak test etc.

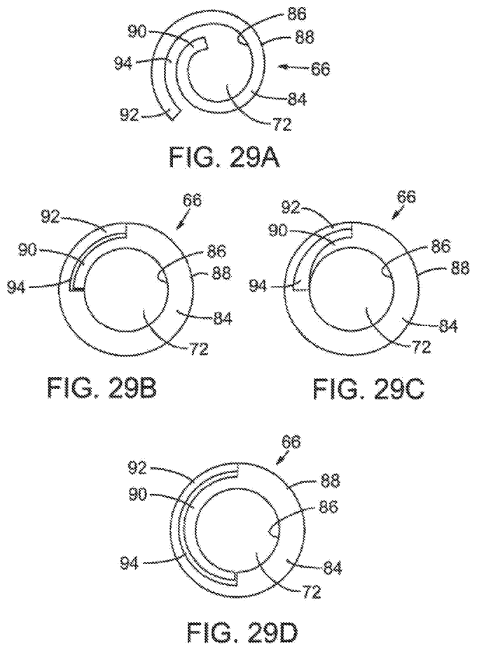

FIGS. 29A-29D show section views of other possible configurations of a sheath 66 for introducing a prosthetic device into a patient's vasculature. The sheath 66 comprises a polymeric tubular layer 84 having an inner surface 86 and an outer surface 88. The thickness of the polymeric tubular layer 84 extends from the inner surface 86 to the outer surface 88. As shown in FIGS. 29B-29D, the polymeric tubular layer 84 can be formed with at least a first angular portion 90 of reduced thickness adjacent the inner surface 86 and a second angular portion 92 of reduced thickness adjacent the outer surface 88, with the second portion 92 at least partially overlapping the first portion 90. FIG. 29A illustrates a similar configuration, where a second portion 92 at least partially overlaps a first portion 90 in a partial coil configuration. In the aspect of FIG. 29A, the second portion 92 and the first portion 90 can have the same thickness.

In preferred aspects, the first and second portions 90, 92 are not adhered to one another. In some aspects, and as best seen in FIG. 29A, there can be a small gap 94 between the first and second portions 90, 92 that can give the sheath 66 the appearance of having two interior lumens 72, 94. FIGS. 29A-29D illustrate the sheath 66 in unexpanded configurations. Preferably, upon expansion of the sheath 66, the ends of the first and second portions 90, 92 abut or are in close proximity to each other to reduce or eliminate any gap between them.

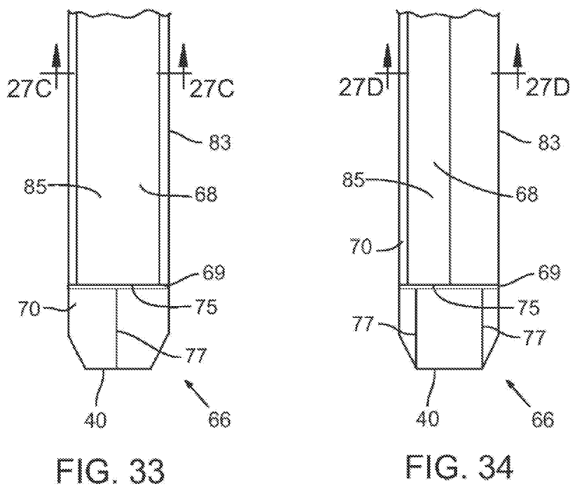

In some aspects, a sheath 66 can comprise a partial slit or score line along at least a portion of its length. For example, as shown in FIG. 33, a sheath 66 can comprise an outer polymeric tubular layer 70 over an inner polymeric liner 68. The inner polymeric layer can extend through a cut in the outer polymeric tubular layer 70 to form a folded region 85 on the outer surface of the outer polymeric tubular layer 70, such as also shown in FIG. 27C. The folded region 85 of the inner layer, in some aspects, terminates before the outer polymeric tubular layer 70 (i.e., the outer polymeric tubular layer 70 is longer than the inner layer). As shown in FIG. 33, in these aspects, the sheath 66 can comprise a partial slit or score line 77 that can extend from the termination (distal end) 75 of the folded region 85 to the distal end 40 of the sheath 66. In some aspects, score line 77 can facilitate expansion of the sheath 66.

Score line 77 can be substantially centrally located with respect to the folded region 85. In alternative aspects, score line 77 can be positioned in other locations relative to the folded region 85. Also, sheath 66 can comprise one or more score lines 77. For example, as shown in FIG. 34, one or more score lines 77 can be peripherally located with respect to the folded region 85. The one or more score lines 77 can be positioned anywhere around the circumference of the outer polymeric tubular layer 70. In aspects comprising a radiopaque marker 69 as seen in FIG. 33, a score line 77 can extend from, for example, the distal end of the radiopaque marker 69 substantially to the distal end 40 of the sheath 66.

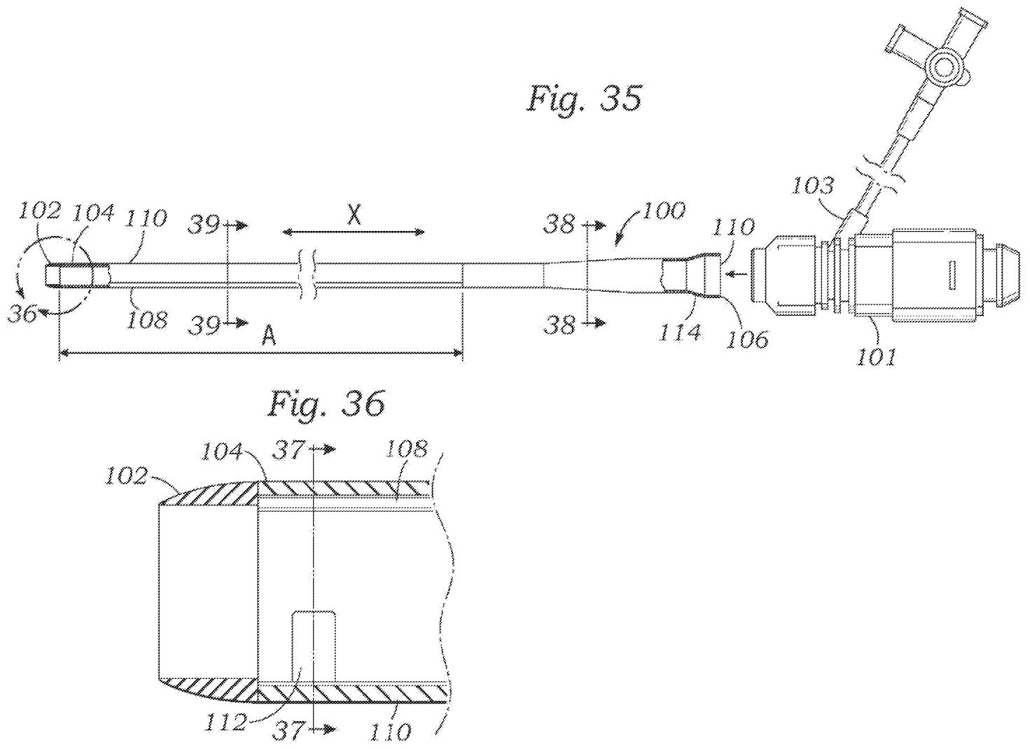

FIGS. 35 and 36 illustrate an expandable sheath 100 according to the present disclosure, which can be used with a delivery apparatus for delivering a prosthetic device, such as a tissue heart valve into a patient. In general, the delivery apparatus can include a steerable guide catheter (also referred to as a flex catheter) a balloon catheter extending through the guide catheter, and a nose catheter extending through the balloon catheter (e.g., as depicted in FIG. 1). It is understood, however, the sheath 100 can refer to any type of the sheath as disclosed herein and that can be used together with the delivery apparatus. The specific configuration of the sheath 100 is not limited to one specific description and can include any configurations disclosed herein. The guide catheter, the balloon catheter, and the nose catheter can be adapted to slide longitudinally relative to each other to facilitate delivery and positioning of the valve at an implantation site in a patient's body. However, it should be noted that the sheath 100 can be used with any type of elongated delivery apparatus used for implanting balloon-expandable prosthetic valves, self-expanding prosthetic valves, and other prosthetic devices. Generally, sheath 100 can be inserted into a vessel (e.g., the femoral or iliac arteries) by passing through the skin of the patient, such that a soft tip portion 102 at the distal end 104 of the sheath 100 is inserted into the vessel. The sheath 100 can also include a proximal flared end portion 114 to facilitate mating with an introducer housing 101 and catheters mentioned above (e.g., the proximal flared end portion 114 can provide a compression fit over the housing tip and/or the proximal flared end portion 114 can be secured to the housing 101 via a nut or other fastening device or by bonding the proximal end of the sheath to the housing). The introducer housing 101 can house one or more valves that form a seal around the outer surface of the delivery apparatus once inserted through the housing, as known in the art. The delivery apparatus can be inserted into and through the sheath 100, allowing the prosthetic device to be advanced through the patient's vasculature and implanted within the patient.

In certain aspects, sheath 100 can include a plurality of layers. For example, sheath 100 can include an inner layer 108 and an outer layer 110 disposed around the inner layer 108. The inner layer 108 can define a lumen through which a delivery apparatus can travel into a patient's vessel in order to deliver, remove, repair, and/or replace a prosthetic device, moving in a direction along the longitudinal axis X. As the prosthetic device passes through the sheath 100, the sheath locally expands from a first resting diameter to a second, expanded diameter to accommodate the prosthetic device. After the prosthetic device passes through a particular location of the sheath 100, each successive expanded portion or segment of the sheath 100 at least partially returns to the smaller resting diameter. In this manner, the sheath 100 can be considered self-expanding in that it does not require the use of a balloon, dilator, and/or obturator to expand.

The inner and outer layers 108, 110 can comprise any suitable materials. Suitable materials for the inner layer 108 include polytetrafluoroethylene (PTFE), ethylene tetrafluoroethylene (ETFE), nylon, polyethylene, polyether block amide (e.g., PEBAX®), and/or combinations thereof. In one specific aspect, the inner layer 108 can comprise a lubricious, low-friction, or hydrophilic material, such as PTFE. Such a low coefficient of friction materials can facilitate the passage of the prosthetic device through the lumen defined by the inner layer 108. In some aspects, the inner layer 108 can have a coefficient of friction of less than about 0.1. Some aspects of a sheath 100 can include a lubricious liner on the inner surface of the inner layer 108. Examples of suitable lubricious liners include materials that can further reduce the coefficient of friction of the inner layer 108, such as PTFE, polyethylene, polyvinylidene fluoride, and combinations thereof. Suitable materials for a lubricious liner also include other materials desirably having a coefficient of friction of about 0.1 or less.

Suitable materials for the outer layer 110 include nylon, polyethylene, PEBAX®, HDPE, polyurethanes (e.g., Tecoflex™), and other medical grade materials. In one aspect, the outer layer 110 can comprise high density polyethylene (HDPE) and Tecoflex™ (or other polyurethane material) extruded as a composite. In some aspects, the Tecoflex™ can act as an adhesive between the inner layer 108 and the outer layer 110 and may only be present along a portion of the inner surface of the outer layer 110. Other suitable materials for the inner and outer layers are also disclosed in U.S. Patent Application Publication No. 2010/0094392, which is incorporated herein by reference.

Additionally, some aspects of a sheath 100 can include an exterior hydrophilic coating on the outer surface of the outer layer 110. Such a hydrophilic coating can facilitate insertion of the sheath 100 into a patient's vessel. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands), as well as other hydrophilic coatings (e.g., PTFE, polyethylene, polyvinylidene fluoride), are also suitable for use with the sheath 100. The elongate tube serving as an outer jacket can then be disposed on the outer layer of the sheath.

Best seen in FIG. 36, the soft tip portion 102 can comprise, in some aspects, low-density polyethylene (LDPE) and can be configured to minimize trauma or damage to the patient's vessels as the sheath is navigated through the vasculature. For example, in some aspects, the soft tip portion 102 can be slightly tapered to facilitate passage through the vessels. The soft tip portion 102 can be secured to the distal end 104 of the sheath 100, such as by thermally bonding the soft tip portion 102 to the inner and outer layers of the sheath 100. Such a soft tip portion 102 can be provided with a lower hardness than the other portions of the sheath 100. In some aspects, the soft tip 102 can have a Shore hardness from about 25 D to about 40 D. The tip portion 102 is configured to be radially expandable to allow a prosthetic device to pass through the distal opening of the sheath 100. For example, the tip portion 102 can be formed with a weakened portion, such as an axially extending score line or perforated line that is configured to split and allow the tip portion to expand radially when the prosthetic device passes through the tip portion (such as shown in the aspects of FIGS. 33 and 34).

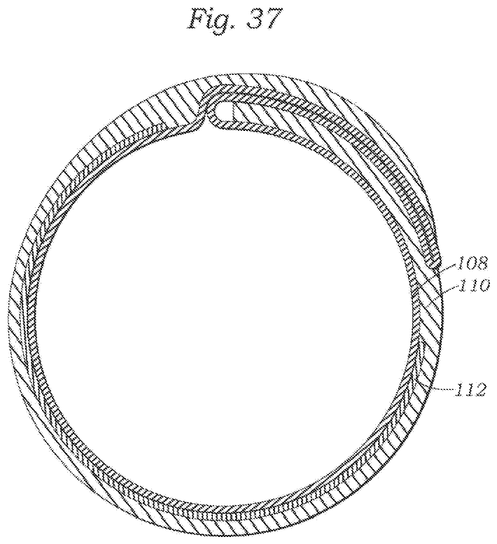

FIG. 37 shows a cross-section view of the sheath 100 taken near the distal end 104 of the sheath 100. As shown in FIGS. 36 and 37, the sheath 100 can include at least one radiopaque filler or marker, such as a discontinuous or C-shaped band 112 positioned near the distal end 104 of the sheath 100. The marker 112 can be associated with the inner and/or outer layers 108, 110 of the sheath 100. For example, as shown in FIG. 37, the marker 112 can be positioned between the inner layer 108 and the outer layer 110. In alternative aspects, the marker 112 can be associated with the outer surface of the outer layer 110. In some aspects, the marker 112 can be embedded or blended within the inner or outer layers 108, 110.

The C-shaped band 112 can serve as a radiopaque marker or filler to enable visibility of the sheath 100 under fluoroscopy during use within a patient. The C-shaped band 112 can comprise any suitable radiopaque material, such as barium sulfite, bismuth trioxide, titanium dioxide, bismuth subcarbonate, platinum, iridium, and combinations thereof. In one specific aspect, the C-shaped band can comprise 90% platinum and 10% iridium. In other aspects, the marker 112 needs not to be a C-shaped band. Other shapes, designs, and configurations are possible. For example, in some aspects, the marker 112 can extend around the entire circumference of the sheath 100. In other aspects, the marker 112 can comprise a plurality of small markers spaced around the sheath 100.

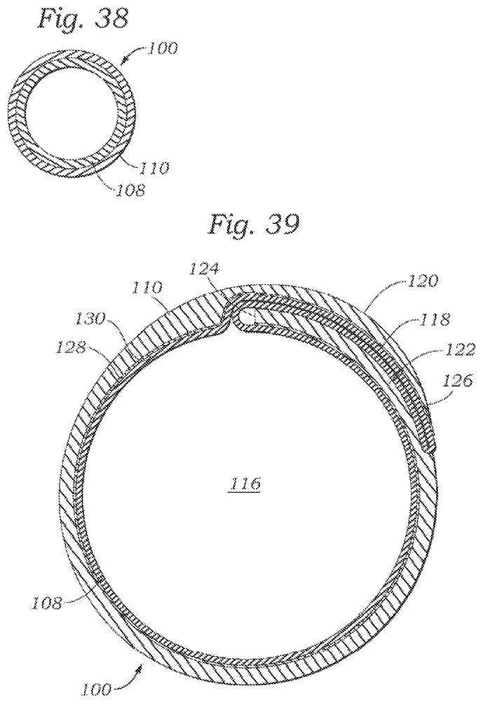

FIGS. 38 and 39 show additional cross sections taken at different points along the sheath 100. FIG. 38 shows a cross-section of a segment of the sheath near the proximal end 106 of the sheath 100, as indicated by line 38-38 in FIG. 35. The sheath 100 at this location can include an inner layer (or liner) 108 and an outer layer 110. At this location, near the proximal end of the sheath, the layers 108, 110 can be substantially tubular, without any slits or folded portions in the layers. By contrast, the layers 108, 110 at different locations along the sheath 100 (e.g., at the point indicated by line 39-39 in FIG. 35) can have a different configuration.

As shown in FIG. 39, the inner layer (liner) 108 can be arranged to form a substantially cylindrical lumen 116 therethrough. Inner liner 108 can include one or more folded portions 118. In the aspect shown in FIG. 39, inner liner 108 is arranged to have one folded portion 118 that can be positioned on either side of the inner layer (liner) 108. The folded portion 118 includes a first fold (e.g., a longitudinally extending fold line) and a second fold and an overlapping portion extending circumferentially therebetween (when the sheath is in an unexpanded configuration). As illustrated in FIG. 39, the folded portion 118 comprises an overlap in a radial direction of at least two thicknesses of the inner layer 108. Inner liner 108 can be continuous in that there are no breaks, slits, or perforations in inner layer 108. Outer layer 110 can be arranged in an overlapping fashion such that an overlapping portion of 120 overlaps at least a part of the folded portion 118 of the inner layer 108. As shown in FIG. 39, the overlapping portion 120 also overlaps an underlaying portion 122 of the outer layer 110. The underlaying portion 122 can be positioned to underlie both the overlapping portion 120 of the outer layer 110, as well as the folded portion 118 of the inner layer 108. Thus, the outer layer 110 can be discontinuous in that it includes a slit or a cut in order to form the overlapping and underlaying portions 120, 122. In other words, a first edge 124 of the outer layer 110 is spaced apart from a second edge 126 of the outer layer 110 so as not to form a continuous layer.

As shown in FIG. 39, the sheath 100 can also include a thin layer of bonding or adhesive layer 128, also referred to as a tie layer, positioned between the inner and outer layers 108, 110. In one aspect, the adhesive layer 128 can comprise a polyurethane material such as Tecoflex™ or etched PTFE tubing. The adhesive layer 128 can be positioned on an inner surface 130 of at least a portion of the outer layer 110 so as to provide adhesion between selected portions of the inner and outer layers 108, 110. For example, the outer layer 110 may only include an adhesive layer 128 around the portion of the inner surface 130 that faces the lumen-forming portion of the inner layer 108. In other words, the adhesive layer 128 can be positioned so that it does not contact the folded portion 118 of the inner layer 108 in some aspects. In other aspects, the adhesive layer 128 can be positioned in different configurations as desired for the particular application. For example, as shown in FIG. 39, the adhesive layer 128 can be positioned along the entire inner surface 130 of the outer layer 110. In an alternative aspect, the adhesive layer can be applied to the outer surface of the inner liner 108 instead of the inner surface of the outer layer. The adhesive layer 128 can be applied to all or selected portions on the inner layer 108; for example, the adhesive layer 128 can be formed only on the portion of the inner layer that faces the lumen-forming portion of the outer layer and not on the folded portion. The configuration of FIG. 39 allows for radial expansion of the sheath 100 as an outwardly directed radial force is applied from within (e.g., by passing a medical device such as a prosthetic heart valve through the lumen 116). As radial force is applied, the folded portion 118 can at least partially separate, straighten, and/or unfold, and/or the overlapping portion 120 and the underlaying portion 122 of the outer layer 110 can slide circumferentially with respect to one another, thereby allowing the diameter of lumen 116 to enlarge.

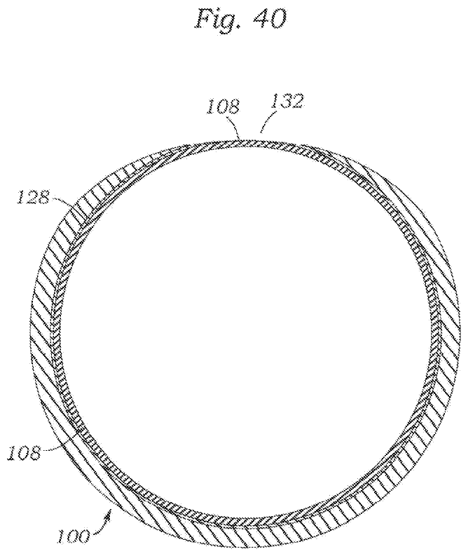

In this manner, the sheath 100 is configured to expand from a resting configuration (FIG. 39) to an expanded configuration shown in FIG. 40. In the expanded configuration, as shown in FIG. 40, an annular gap 132 can form between the longitudinal edges of the overlapping portion 120 and the underlaying portion 122 of the outer layer 110. As the sheath 100 expands at a particular location (i.e., locally expands at the location of the passing prosthetic device), the overlapping portion 120 of the outer layer 110 can move circumferentially with respect to the underlaying portion 122 as the folded portion 118 of the inner layer 108 unfolds. This movement can be facilitated by the use of a low-friction material for inner layer 108, such as PTFE. Further, the folded portion 118 can at least partially separate and/or unfold to accommodate a medical device having a diameter larger than that of lumen 116 in the resting configuration. As shown in FIG. 40, in some aspects, the folded portion of the inner layer 108 can completely unfold so that the inner layer 108 forms a cylindrical tube at the location of the expanded configuration.

The sheath 100 can be configured such that it locally expands at a particular location corresponding to the location of the medical device along the length of the lumen 116, and then locally contracts once the medical device has passed that particular location. Thus, a bulge may be visible, traveling longitudinally along the length of the sheath as a medical device is introduced through the sheath, representing continuous local expansion and contraction as the device travels the length of the sheath 100. In some aspects, each segment of the sheath 100 can locally contract after removal of any radial outward force such that it regains the original resting diameter of the lumen 116. In some aspects, each segment of the sheath 100 can locally contract after removal of any radial outward force such that it at least partially returns to the original resting diameter of the lumen 116.

The layers 108, 110 of sheath 100 can be configured, as shown in FIG. 39 along at least a portion of the length of the sheath 100. In some aspects, the layers 108, 110 can be configured as shown in FIG. 39 along the length A (FIG. 35) extending from a location adjacent the soft tip portion 102 to a location closer to the proximal end 106 of the sheath 100. In this matter, the sheath is expandable and contractable only along a portion of the length of the sheath corresponding to length A (which typically corresponds to the section of the sheath inserted into the narrowest section of the patient's vasculature).

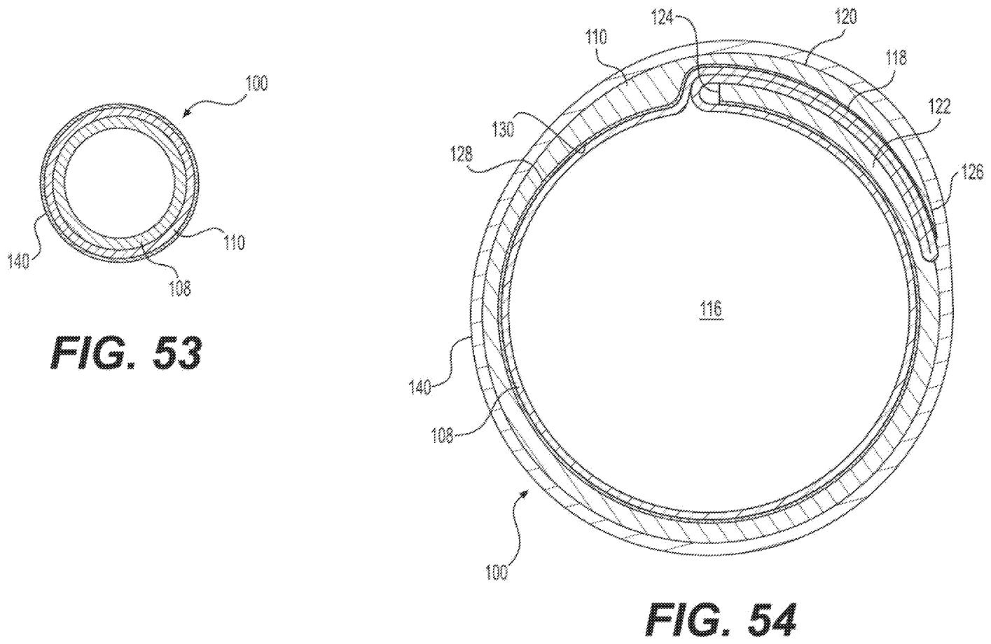

FIGS. 53 and 54 show additional cross sections taken at different points along the sheath 100 that include the disclosed herein outer jacket (elongated tube) 140 that behaves as an outer jacket of the sheath. Similar to FIG. 38, FIG. 53 shows a cross-section of a segment of the sheath near the proximal end 106 of the sheath 100, as indicated by line 38-38 in FIG. 35. The sheath 100 at this location can comprise an inner layer (liner) 108, outer layer 110, adhesive layer 128, and a second outer layer (the outer jacket) 140. In this exemplary aspect, at this location, near the proximal end of the sheath, the layers 108, 110, and 140 can be substantially tubular, without any slits or folded portions in the layers. By contrast, the layers 108, 110 at different locations along the sheath 100 (e.g., at the point indicated by line 39-39 in FIG. 35) can have a different configuration, while the second outer layer (the outer jacket) 140 maintains a substantially tubular shape, without slits or folds. It is understood that outer jacket 140, as shown herein, can have any composition and characteristics of the elongated tube disclosed above.

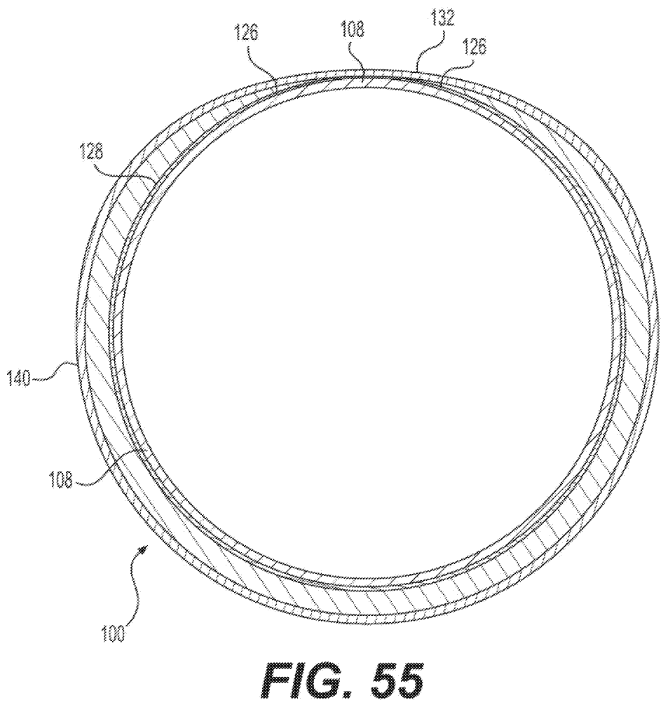

As shown in FIG. 54, and described above with respect to FIG. 39, the inner layer (liner) 108 can be arranged to form a substantially cylindrical lumen 116 extending therethrough. The inner layer 108 can include one or more folded portions 118. The outer layer 110 can be arranged in an overlapping fashion such that an overlapping portion 120 overlaps at least a part of the folded portion 118 of the inner layer 108 as well as the underlaying portion 122 (positioned to underlie the folded portion 118 of the inner layer 108) when the sheath is unexpanded. The sheath 100 is configured to locally expand from an unexpanded configuration in which the lumen 116 has a first diameter to an expanded configuration in which the lumen 116 has a second diameter larger than the first diameter. The sheath 100 expands in response to an outwardly directed radial force exerted by a medical device against the inner layer 108 as it passes through the lumen 116. During expansion, the first fold/folded edge moves closer to the second fold/folded edge to shorten the folded portion 118. As shown in FIG. 55, in some aspects, the folded portion 118 of the inner layer 108 can completely unfold so that the inner layer (liner) 108 forms a cylindrical tube at the location of the expanded configuration. When the sheath is expanded, a portion of the inner layer 108 extends through the opening/gap provided in the outer layer 110, where the opening is formed by the longitudinally extending edge of the overlapping portion 120 and a longitudinally extending edge of the underlaying portion 122. As the prosthetic device passes, the sheath 100 then locally contracts at least partially back to the unexpanded configuration.

As described above, the sheath 100 includes an inner layer 108. The inner layer 108 can be surface-treated, such as by plasma etching, chemical etching, or other suitable methods of surface treatment. In certain aspects, the inner liner 108 is selectively etched, forming various etched and nonetched portions, as disclosed above. By treating the surface of the inner layer 108, the outer surface of the inner liner 108 can have areas with altered surface angles that can provide better adhesion between the inner layer 108 and the outer layer 110. As described above, the inner liner 108 can comprise polytetrafluoroethylene (PTFE), polyimide, polyetheretherketone (PEEK), polyurethane, nylon, polyethylene, polyamide, or combinations thereof. In an example, sheath 100, the inner layer 108 is composed of an etched PTFE material. It is contemplated that the inner layer 108 can have a fully etched outer surface or a partially etched outer surface. When partially etched, the unetched portions of the outer surface of the inner liner 108 can extend longitudinally along a length of the inner layer 108 and/or circumferentially around the circumference of the inner layer 108. For example, the desired unetched location on the inner layer 108 can be masked or otherwise covered during the etching process to prevent etching at that location. It is also contemplated that the entire outer surface of the inner liner 108 can be etched and the etching removed at the desired locations of the unetched surface.

In an example, sheath 100, unetched portions are provided along those surfaces of the inner layer 108 that come into contact with the outer surface of the outer layer 110. That is, those portions of the inner layer 108, excluding the tie layer 128, would not include etching. For example, it is contemplated that etching is not included between the inner surface of the folded portion 118 of the inner layer 108 and the underlaying portion 122 of the outer layer 110. By excluding etching on the portions where the inner layer 108 and the outer surface of the outer layer 110 are in direct contact helps to facilitate release of the inner surface of the folded portion 118 and the outer layer 110 during expansion of the sheath 100.

The wall thickness of the inner liner can vary, but in some examples, the wall thickness of the inner layer 108 ranges between about 0.002 inches and about 0.006 inches (including about 0.002 inches, about 0.003 inches, about 0.004 inches, about 0.005 inches, about 0.006 inches). In other examples, the wall thickness of the inner layer ranges between about 0.003 includes and about 0.005 inches. In a further example, the wall thickness of the inner layer (liner) 108 ranges between about 0.0035 inches and about 0.0045 inches (including about 0.0035 inches, about 0.0040 inches, about 0.0045 inches).

As described above, the sheath 100 includes an outer layer 110 exerting a radially inward force on the inner layer 108. In general, the outer layer 110 can comprise a polymeric material. As described above, the outer layer 110 can be comprised of PTFE, polyimide, PEEK, polyurethane, nylon, polyethylene, polypropylene, polyamide, polyether block amides, polyether block ester copolymer, thermoset silicone, latex, poly-isoprene rubbers, high density polyethylene (HDPE), Tecoflex™, or combinations thereof. In an exemplary aspect, the inner layer 108 can comprise PTFE, and the outer layer 110 can comprise a combination of HDPE and Tecoflex™. The outer layer 110 can have a wall thickness ranging between about 0.007 inches and about 0.013 inches (including 0.007 inches, about 0.008 inches, about 0.009 inches, about 0.010 inches, about 0.011 inches, about 0.012 inches, about 0.013 inches),In another example, the outer layer 110 can have a wall thickness ranging between about 0.008 inches and about 0.012 inches. In another example, the outer layer 110 can have a wall thickness ranging between about 0.009 inches and about 0.011 inches.

As described above, the sheath 100 includes an outer jacket 140 that extends over and envelopes the outer layer 110. While the outer layer 110 can be discontinuous in that it includes a slit or a cut in order to form the overlapping and underlaying portions 120, 122 as described above, the outer jacket 140 comprises a continuous outer layer covering the inner and outer layers 108, 110. It is understood that the outer jacket is formed by the disclosed above elongated tube having a disclosed composition and characteristics.

Generally, the outer jacket 140 has a relatively low tensile modules compared to the inner and outer layers 108, 110. In certain aspects and as disclosed above, the outer jacket exhibits an elongation at break of ranging between about 40% and about 800% (including about 40%, about 50%, about 60%, about 70%, about 80%, about 90%, about 100%, about 150%, about 200%, about 250%, about 300%, about 350%, about 400%, about 450%, about 500%, about 550%, about 600%, about 650%, about 700%, about 800%).

The outer jacket 140 in this aspect, can have a first polymer layer and a second polymer layer, similar to the aspects disclosed above. For example, the first polymer layer can comprise a first compound composition comprising from greater than 0 wt % to less than 100 wt % of a polymer comprising a polyether block amide, a polyurethane, or a combination thereof; less than about 65 wt % of an inorganic filler based on a total weight of the first compound composition; and up to about 20 wt % of a solid lubricant filler based on a total weight of the first compound composition. In yet further aspects, the first polymer layer can also comprise at least one tackiness reducing compound in an amount from about 1wt % to about 20 wt %. In still further aspects, the solid lubricant filler can comprise one or more of graphene, reduced graphene oxide, carbon black, boron nitride, silicones, talc, polytetrafluorethylene (PTFE), fluorinated ethylene propylene, and the like. In yet further aspects, the inorganic filler can comprise bismuth oxychloride, barium sulfate, bismuth subcarbonate, calcium carbonate, aluminum trihydrate, barite, kaolin clay, limestone, or any combination thereof. In still further aspects, and as disclosed above, the least one tackiness reducing compound comprises ProPell™.

In yet further aspects, the outer jacket 140 can comprise a second polymer layer. In such aspects, the second polymer layer can have any composition, as disclosed above. For example, and without limitation, the second polymer layer can comprise a second compound composition comprising from 0 wt % to 100 wt % of a second polymer comprising polyether block amide, a polyurethane, or a composition thereof. In still further aspects, the second compound composition is substantially free of inorganic filler. While in other aspects, the second compound composition is substantially free of the solid lubricant filler.

The outer jacket 140 can comprise the first and/or the second polymer layer with a wall thickness ranging between about 1 mil to about 5 mils, including exemplary values of about 1.5 mils, about 2 mils, about 2.5 mils, about 3 mils, about 3.5 mils, about 4 mils, about 4.5 mils, and about 4.9 mils. The wall thickness is measured radially between the inner surface of the outer jacket 140 and the outer surface of the outer jacket 140.

In alternative aspects, the first (and/or second) polymer layer composition and/or wall thickness can change along the length of the outer jacket 140. For example, the outer jacket (elongated tube) 140 can be provided with one or more segments where the composition and/or thickness changes from segment to segment. In an example aspect, the Durometer rating of the composition changes along the length of the outer jacket 140 such that segments near the proximal end comprise a stiffer material or combination of materials, while segments near the distal end comprise a softer material or combination of materials. Similarly, the wall thickness of the outer jacket 140 the wall thickness of the outer jacket 140 in segments near the proximal end can be thicker/greater than the wall thickness of the outer jacket 140 near the distal end.

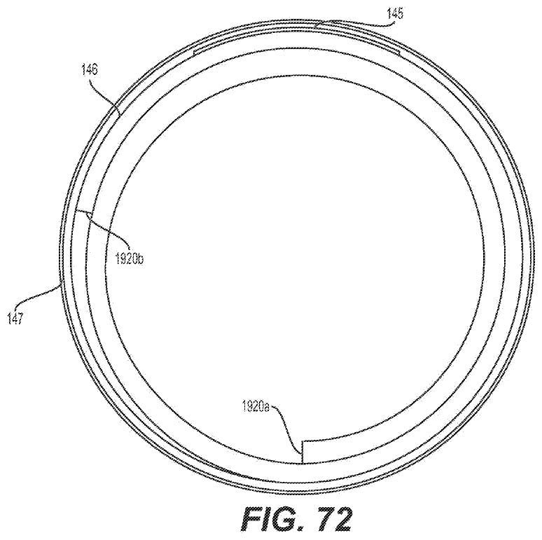

As illustrated in FIG. 71-72, the outer jacket 140 includes one or more axial reinforcing members 145 that extend longitudinally along all or a portion of the outer jacket 140. The reinforcing member 145 helps to prevent axial bunching of the outer jacket 140 during insertion into the patient's vasculature while not sacrificing the low radial expansion force of the outer jacket 140.

As illustrated in FIG. 35, the sheath 100 can include a tapered segment adjacent the flared end portion 114 at the proximal end of the sheath 100. Referred to as a strain relief section, the tapered segment and the flared end portion 114 help ease the transition between the smaller diameter portion of the sheath 100 and the housing 101. The thickness and/or composition of the outer jacket 140 can be adjusted to improve the performance of the strain relief section and to reduce the push force as disclosed above.

The outer jacket (or elongated tube) 140 can be bonded to the outer layer 110 to prevent the outer jacket (elongated tube) 140 from sliding over the outer layer 110 and “bunching up” in response to the friction forces applied by the surrounding tissue during the insertion of the sheath 100 into the patient's vasculature. For example, the outer jacket (the elongated tube) 140 can be bonded at the proximal end and/or distal end of the outer layer 110. At the proximal and distal ends, the outer jacket 140 can be bonded to the outer layer 110 around the full circumference of the outer layer. At the distal end of the sheath 100, the outer jacket (the elongated tube) 140 can alternatively be bonded to the inner layer 108. For example, the outer jacket (the elongated tube) 140 can be bonded to the distal end surface of the inner layer 108.

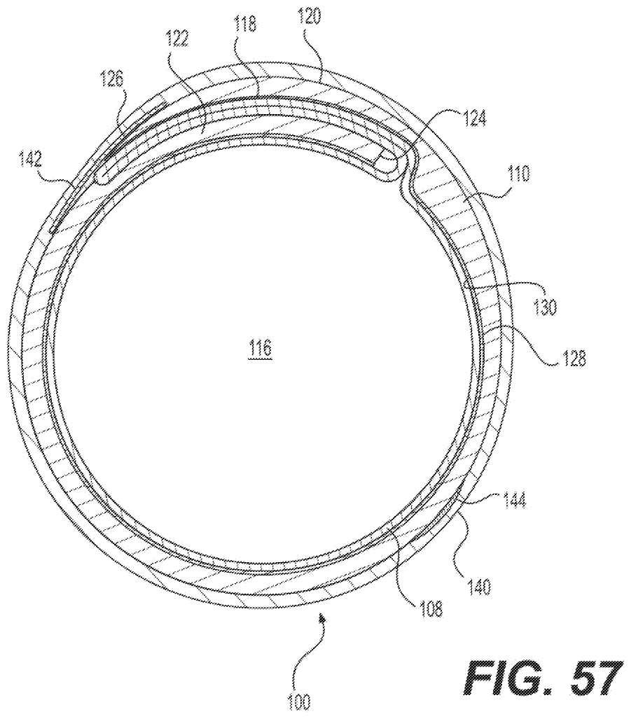

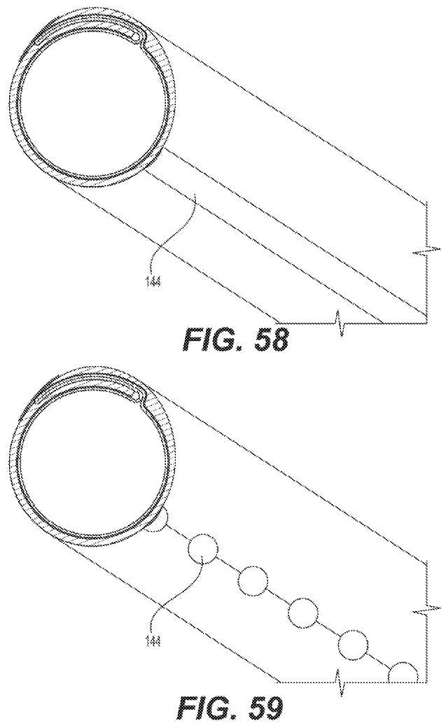

As illustrated in FIG. 57, the outer jacket (elongated tube) 140 can be bonded 144 to the outer layer 110 at a circumferential location opposite the folded portion 118 of the inner layer 108. As provided in FIGS. 58-59, the bond 144 can be spot bonds or linear bond lines extending along all or a portion of the outer layer 110. As provided in FIG. 57, the bond 144 line/spot will also have a width, extending circumferentially around the outer layer 110. For example, the bond line can cover about 5° to about 90° of the circumference of the outer layer 110 (including about 5°, about 10°, about 15°, about 20°, about 25°, about 30°, about 35°, about 40°, about 45°, about 50°, about 55°, about 60°, about 65°, about 70°, about 75°, about 80°, about 85°, about 90°).

The outer jacket (elongated tube) 140 can be bonded to the outer layer 110 and/or inner layer 108 using any mechanical and/or chemical (e.g., adhesive) fastener known in the art. In one example, sheath 100, the outer jacket 140 and the outer layer 110 and/or inner layer 108 may have similar melting temperatures. Accordingly, an example bonding method includes a thermally bonded coupling between the outer jacket (elongated tube) 140, the outer layer 110, and/or inner layer 108. For example, the bond between the outer jacket 140 and the outer layer 110 can be achieved by laser welding and/or a heat compression (e.g., using a heat compression jaw), allowing the location of the bond line to be closely controlled.

As shown in FIG. 54, and described above with respect to FIG. 39, an adhesive layer 128 (e.g., a tie layer) is provided between the inner layer 108 and the outer layer 110 to at least partially adhere the inner layer 108 to the outer layer 110. That is, the adhesive layer 128 is selectively provided/located between the inner layer 108 and the outer layer 110 to bond the inner and outer layers 108, 110 at the selected locations of the adhesive layer 128.

As illustrated in FIGS. 54 (and FIG. 39), the adhesive layer 128 is provided on the outer surface of the inner liner 108 and/or the inner surface 130 of the outer layer 110. For example, the adhesive layer 128 can be provided partially or entirely around the outer surface of the inner liner 108. Additionally, or alternatively, the adhesive layer 128 can be provided partially and/or entirely around the inner surface 130 of the outer layer 110. As illustrated in FIG. 54, the adhesive layer 128 extends between the outer layer 110 and the overlapping folded portion 118 of the inner layer 108. That is, the adhesive layer 128 extends between the outer surface of the folded portion 118 of the inner layer 108 and the corresponding inner surface of the overlapping portion 120 of the outer layer 110. As illustrated in FIG. 54, the adhesive layer 128 does not extend between an inner surface of the overlapping folded portion 118 of the inner layer 108 and a corresponding surface of the underlaying portion 122 of the outer surface of the outer layer 110. Excluding the adhesive layer 128 on the portion of the sheath between the inner surface of the folded portion 118 and the underlaying portion 122 facilitates expansion of the sheath and prevents undesirable bonding/sticking between the inner and outer layers 108, 110 at this location.

The adhesive layer 128 can comprise a material having a Shore A hardness (durometer) less than about 90 A. For example, the adhesive layer 128 can comprise a thermoplastic polyurethane such as an aliphatic polyether-based thermoplastic polyurethane (TPU). An example of TPU includes Tecoflex™ 80A. The adhesive layer 128 can also be composed of an aromatic polyether or polyesters-based thermoplastic polyurethane such as, for example, Pellethane™ 80A. The adhesive layer can also be composed of a polyolefin or polyamide, including, for example, a polyolefin (PE, PP, or EVA) modified with maleic anhydride such as an Orevac™ resin.

The thickness (wall thickness) of the adhesive layer 128 can vary, but in some examples, the wall thickness of the adhesive layer ranges between about 0.002 inches and about 0.005 inches (including about 0.002 inches, about 0.003 inches, about 0.004 inches, about 0.005 inches). In other examples, the wall thickness of the adhesive layer 128 ranges between about 0.0025 and about 0.0040 (including about 0.0025 inches, about 0.0030 inches, about 0.0035 inches, about 0.0040 inches). In a further example, the wall thickness of the adhesive layer 128 ranges between about 0.0025 inches and about 0.0035 inches (including about 0.0025 inches, about 0.0030 inches, about 0.0035 inches).

In some examples, the sheath 100 can include a lubricant to reduce friction and facilitate expansion/contraction between the outer layer 110 and the outer jacket 140. The lubricant 142 allows the outer layer 110 and inner layer 108 to unroll easily under the outer jacket (elongated tube) 140, ensuring that the hemostasis and atraumatic benefits achieved by the addition of an outer jacket 140, do not compromise the push force performance of the sheath 100. That is, the lubricant 142 reduced the push force necessary to move the prosthetic device through the lumen 116 of the inner layer 108 during the delivery of the prosthetic device and the corresponding local expansion of the sheath 100.

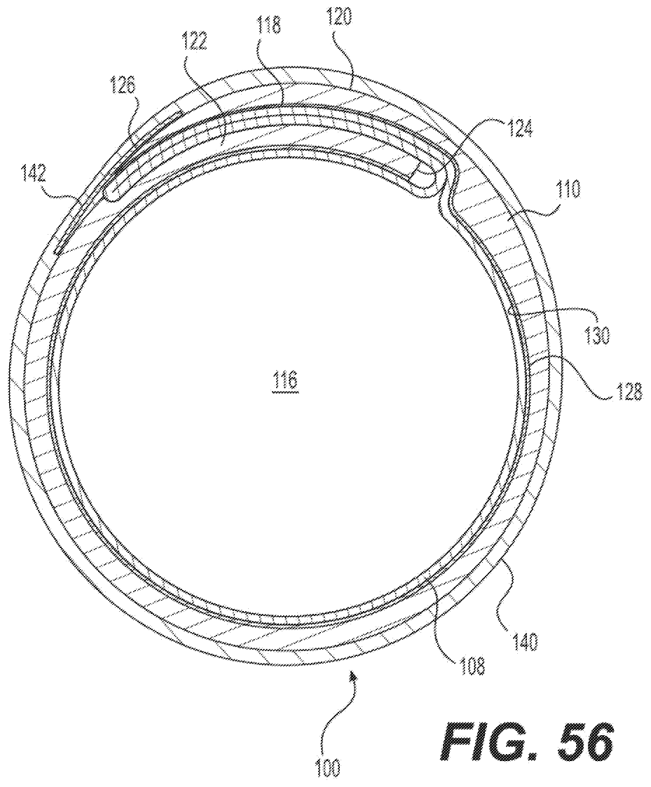

As illustrated in FIG. 56, the lubricant 142 can be selectively applied along an outer surface of the outer layer 110 proximate to the longitudinally extending edge 126 of the overlapping portion 120. In some examples, a portion of the folded portion 118 of the inner layer 108 extends beyond the longitudinally extending edge 126 of the overlapping portion 120 and along an outer surface of the outer layer 110. In this example, the lubricant 142 is also provided along the protruding portion, the folded portion 118 of the inner layer 108 extending along the outer surface of the outer layer 110 (beyond the edge 126). In this location, the lubricant 142 also reduces friction between the outer jacket 140 and the inner layer 108 during expansion of the sheath 100. As illustrated in FIG. 56, the lubricant 142 extends around the circumference of the outer layer 110 beyond the protruding portion of the folded portion 118.

The lubricant is applied as a band (or spot) that extends both circumferentially and longitudinally along the outer layer 110 (and the protruding portion of the inner layer 108). In an example sheath 100, the lubricant 142 is applied as a band that extends both circumferentially around the outer layer 110 and longitudinally along a length of the outer layer 110. To prevent migration, the lubricant 142 can be composed of a heat-curable material, e.g., a material curable at room temperature. As a result, the material can be applied to a desired location along the outer layer 110 and does not migrate during assembly and/or use of the sheath 100. The lubricant 142 can be composed of a medical-grade lubricant, such as silicone. Example lubricants include medical-grade curable silicone lubricants, including a platinum catalyzed thermal curing silicone lubricant such as NuSil™ MED10-6670 (heat-curable), a PTFE lubricant such as Duraglide™ (curable at room temperature) and/or CHRISTO-LUBE™.

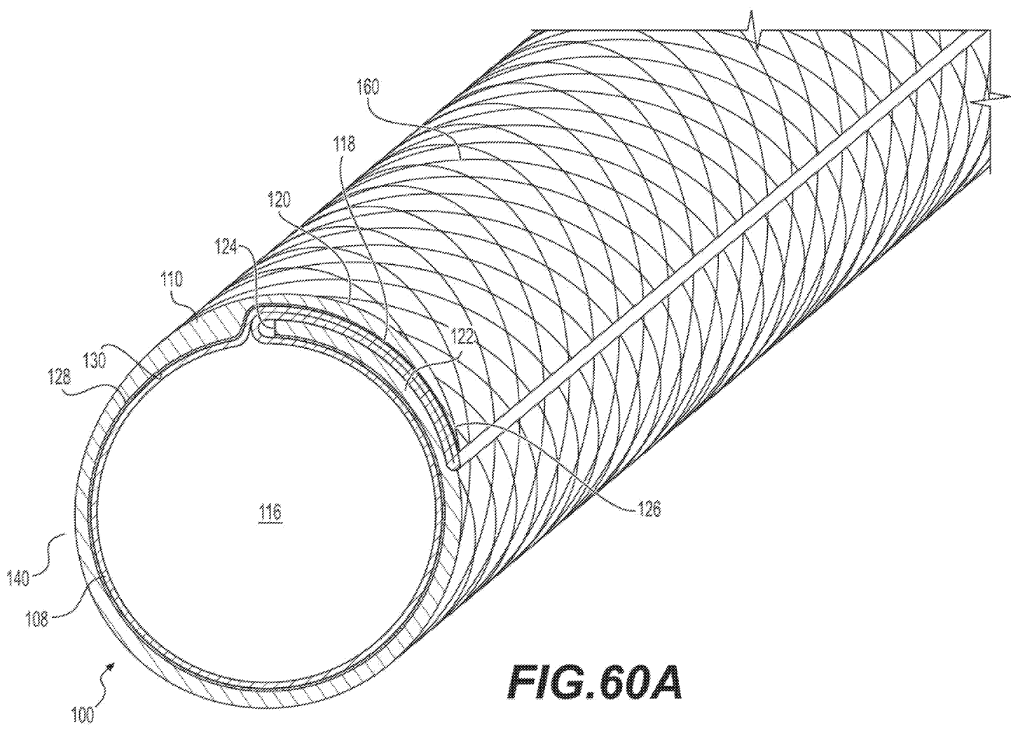

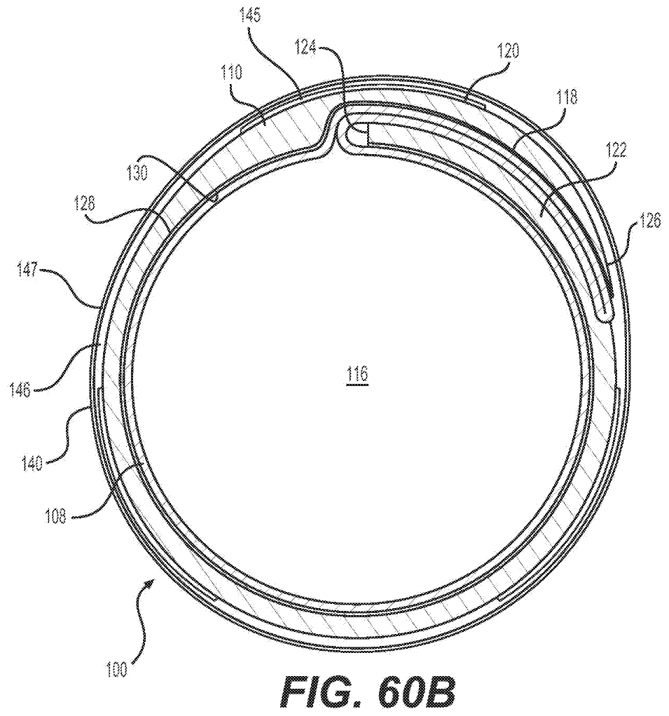

FIG. 60A illustrates an additional aspect of the sheath 100 of FIG. 35. In this example, the sheath 100 can include a coiled wire 160, or coiled wire mesh, along a length of the sheath 100. The coiled wire 160 provides uniform bending of the sheath and prevents kinking. The coiled wire 160 can be embedded in the outer layer 110. For example, the coiled wire 160 can be co-extruded with the outer layer 110. Alternatively, the coiled wire 160 can be provided between the outer layer and the adhesive layer 128. In another example, the coiled wire 160 is embedded, at least partially, within both the outer layer 110 and the adhesive layer 128. For example, the coiled wire 160 can be provided on an outer surface of the adhesive layer 128, and the outer layer 110 is reflowed over.

As illustrated in FIG. 60A, the coiled wire 160 defines a helical-shaped path around the longitudinal axis of the sheath 100. The example of coiled wire 160 includes an overlapping helical-shaped path around the longitudinal axis of the sheath 100, resulting in a continuous diamond-shaped pattern along the length of the sheath.

The coiled wire 160 can be comprised of a metal or a polymer wire. For example, the coiled wire 160 can be composed of PET, PEEK, stainless steel, and/or nitinol. The coiled wire 160 can be comprised of a flat wire, a round wire, or a combination thereof. The individual wires of the coiled wire 160 can have a diameter/thickness ranging between about 0.002 inches and about 0.008 inches (including about 0.002 inches, about 0.003 inches, about 0.004 inches, about 0.005 inches, about 0.006 inches, about 0.007 inches, about 0.008 inches). In another example, the individual wires of the coiled wire 160 can have a diameter/thickness ranging between about 0.004 inches and about 0.007 inches. In a further example, the individual wires of the coiled wire 160 can have a diameter/thickness of about 0.006 inches. The pitch/distance between adjacent coils of the coiled wire 160 can correspond to the diameter/thickness of the coiled wire. For example, where the diameter/thickness of a single coil of the coiled wire is about 0.006 inches, the spacing/pitch between the wire and the next adjacent coiled wire is about 0.006 inches.

Methods of using the sheath of FIGS. 53-60A include first inserting the expandable sheath 100 into the vasculature of a subject and advancing a prosthetic device through the inner lumen 116 of the inner layer 108/sheath 100. The prosthetic device applies an outwardly directed radial force on the inner layer 108 of the expandable sheath 100. In some aspects, the outwardly directed radial force is transmitted through the inner layer 108, the adhesive layer 128, and the outer layer 110. The lumen 116 of the sheath 100 expands at the axial location of the prosthetic device due to the outwardly directed radial force exerted by a prosthetic device against an inner surface of the lumen during advancement. During expansion of the lumen 116, the first fold (folded edge) of the folded portion 118 is moved circumferentially closer to the second fold (folded edge), shortening the overlapping portion of the folded portion 118 that extends circumferentially between the first and second folds, thereby increasing the circumference of the lumen 116.

As the sheath 100 expands at a particular location (i.e., locally expands at the location of the passing prosthetic device), the overlapping portion 120 of the outer layer 110 can move circumferentially with respect to the underlaying portion 122 as the folded portion 118 of the inner layer 108 least partially separate and/or unfold, causing the elongate gap(s) 132 provided in the outer layer 110 to widen/expand. The sheath thereby expands to accommodate a medical device having a diameter larger than that of lumen 116 in the resting (unexpanded) configuration. As shown in FIG. 55, in some aspects, the folded portion of the inner layer 108 can completely unfold so that the inner layer 108 forms a cylindrical tube at the location of the expanded configuration. As illustrated in FIGS. 54 and 55, the elongate gap 132 is generally aligned with the longitudinal axis of the lumen 116 such that during expansion, the unfolded portion of the inner layer 108 expands into the gap 132.

In an unexpanded configuration, the sheath 100 can have an outer diameter less than about 0.30 inches (including less than about 0.29 inches, less than about 0.28 inches, less than about 0.27 inches, less than about 0.26 inches, less than about 0.25 inches, less than about 0.24 inches). Preferably, the unexpanded sheath has an outer diameter ranging between about 0.24 inches and about 0.26 inches. In a fully expanded configuration, the sheath 100 can have an inner diameter greater than 0.300 inches. Preferably, the expanded sheath 100 has an inner diameter ranging between 0.325 inches and 0.400 inches (including about 0.325 inches, about 0.327 inches, about 0.330 inches, about 0.333 inches, about 0.336 inches, about 0.340 inches, about 0.345 inches, about 0.350 inches, about 0.355 inches, about 0.360 inches, about 0.365 inches, about 0.370 inches, about 0.375 inches, about 0.380 inches, about 0.385 inches, about 0.390 inches, and about 0.395 inches).

As described above, the inner and outer layers 108, 110 can be bonded together using an adhesive layer 128. The adhesive layer 128 prevents movement, both longitudinal and radial, between the inner and outer layers 108, 110. As a result, expansion of the sheath 100 can be limited to only those regions excluding the adhesive layer 128. For example, as illustrated in FIG. 54, because the adhesive layer 128 is not provided between the inner surface of the folded portion 118 and the underlaying portion 122 of the outer layer, expansion of the sheath results in the inner surface of the folded portion extending into the gap 132 created between the first and second edges 124, 126 of the expanded outer layer 110.

Once the prosthetic device is passed through the lumen 116 (or a particular location along the lumen), the lumen 116 of the sheath can at least partially contracts back to an unexpanded configuration. The outer layer 110 can exert an inwardly directed radial force on the inner layer 108 urging it back to its original folded configuration. Similarly, if a coiled wire 160 was included, the coiled wire can exert an inwardly directed radial force on the outer layer 110 and the inner layer 108 urging them back towards an unexpanded configuration. In still further aspects, the outer jacket 140 can exert an inwardly directed radial force on the outer layer 110 and the inner layer 108 urging them back to an unexpanded configuration.

The prosthetic device can be delivered through the distal end of the sheath 100 to the delivery site within the patient. The prosthetic device can include a self-expanding heart valve or a stent-mounted heart valve. The heart valve can be extended through the distal end of the elongate lumen 116 at the delivery site. Once outside the lumen 116, the heart valve can be expanded, and the sheath 100 removed from the treatment site.

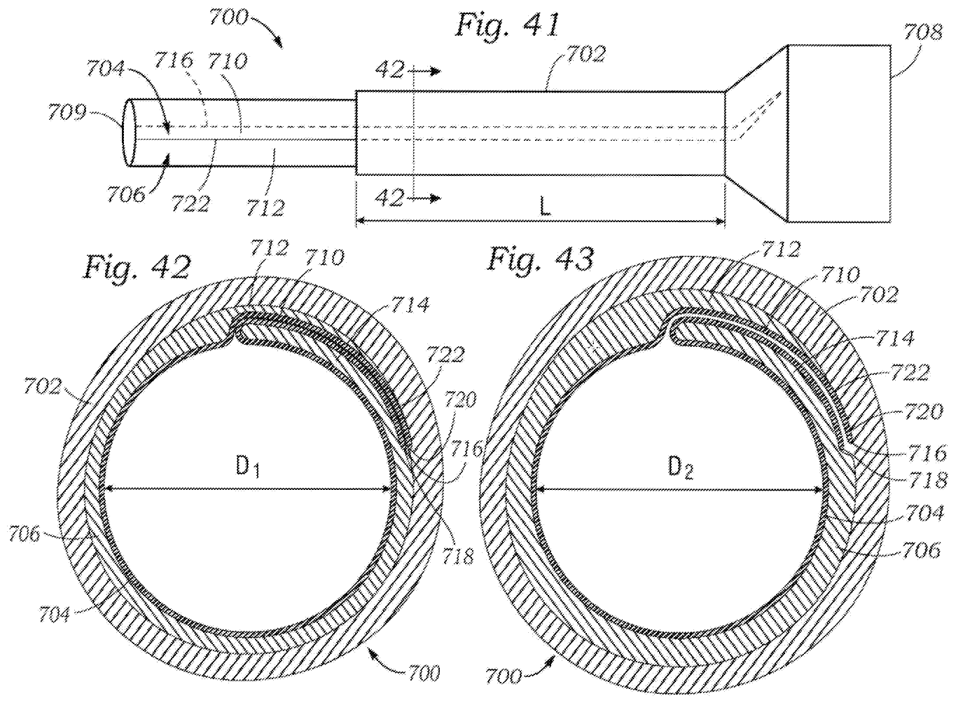

FIGS. 41-49 illustrate additional aspects and variations on the general sheath 100 described above. It is to be understood that the variations (e.g., materials and alternate configurations) described above with reference to any previously disclosed figures and aspects can also apply to the aspects shown in FIGS. 41-49 and vice versa.

FIGS. 41-43 illustrate a sheath 700 that additionally includes a strain relief cover comprising disclosed herein elongated tube 702 positioned around at least a part of an inner layer 704 and outer layer 706. As shown in FIG. 41, the elongated tube (the outer jacket or a strain relief jacket) 702 can extend for a length L along at least a portion of the main body of the sheath 700. In some aspects, the elongated tube 702 can extend from the proximal end 708 of the sheath 700 and towards the distal end 709 of the sheath. In some aspects, the elongated tube 702 extends only part way down the length of the sheath 700. In alternate aspects, the elongated tube 702 can extend to a point adjacent the distal end 709 or can extend all the way to the distal end 709 of sheath 700. Furthermore, the elongated tube 702 need not extend all the way to the proximal end 708 of the sheath 700. In some aspects, the elongated tube 702 can extend only part way towards the proximal end 708. In some aspects, the longitudinal length L of the elongated tube 702 can range from about 10 cm to the entire length of the sheath 700.

As shown in FIGS. 42 and 43, the elongated tube 702 can be a continuous tubular layer without slits or other discontinuities. The elongated tube 702 can be positioned to surround the entire circumference of outer layer 706 and can extend longitudinally along any portion of the length of the sheath 700. The elongated tube 702 can comprise any of the disclosed above compositions.

The elongated tube 702 can, in some aspects, provide hemostasis (e.g., prevent blood loss during implantation of the prosthetic device). For example, the elongated tube 702 can be sized or configured to form a seal with the patient's artery when inserted, such that blood is substantially prevented from flowing between the elongated tube 702 and the vessel wall. The elongated tube 702 can be inserted such that it passes the arteriotomy. For example, in aspects where the elongated tube 702 does not extend all the way to the distal end 709 of the sheath 700, the elongated tube 702 can extend distally far enough such that when the sheath 700 is fully inserted into the patient, at least part of the elastic outer cover extends through the ateriotomoy site.

The elongated tube 702 can be configured to expand as the sheath expands, as shown in the expanded configuration in FIG. 43.

FIG. 42 shows a cross-section of the sheath 700 in a resting configuration having an inner diameter D₁. FIG. 43 shows a cross-section of the sheath 700 in an expanded configuration, having an inner diameter D₂, where D₂ is greater than D₁. Similar to the aspect of FIGS. 35-40, the sheath 700 can include an inner layer 704 having a folded portion 710, and an outer layer 706 having an overlapping portion 712 and an underlaying portion 714. The overlapping portion 712 overlaps at least a portion of the folded portion 710 of the inner layer, and the underlaying portion 714 underlies at least a portion of the folded portion 710. As shown in FIGS. 42-43, in some aspects, the overlapping portion 712 does not overlap the entire folded portion 710 of the inner layer 704, and thus a portion of the folded portion 710 can be directly adjacent to the elongated tube 702 in locations where the elongated tube 702 is present. In locations where the elongated tube 702 is not present, part of the folded portion 710 may be visible from the outside of the sheath 700, as seen in FIG. 41. In these aspects, the sheath 700 can include a longitudinal seam 722 where the overlapping portion 712 terminates at the folded portion 710. In use, the sheath can be positioned such that the seam 722 is posterior to the point of the sheath that is 180 degrees from the seam 722 (e.g., facing downward in the view of FIG. 41). The seam 722 can also be seen in FIG. 41, which shows that the seam 722 need not extend the entire length of the sheath. In some aspects, the proximal end portion of the sheath includes two layers without a folded portion (e.g., similar to FIG. 38), while the distal end portion of the sheath includes two layers with a folded portion (e.g., similar to FIG. 39). In some aspects, the seam 722 can end at a transition point between portions of the sheath having a folded inner layer and portions of the sheath not having a folded inner layer.

In some aspects, the folded portion 710 can include a weakened portion 716, such as a longitudinal perforation, score line, and/or slit along at least a portion of the length of the inner layer 704. The slit can allow for two adjacent ends 718, 720 of the folded portion 710, to move relative to one another as the sheath 700 expands to the expanded configuration shown in FIG. 43. As a device having an outer diameter device larger than the initial resting inner diameter of the sheath 700 is inserted through the sheath 700, the device can cause local expansion of the sheath 700 and cause the sheath 700 to expand at the partial score or split line location. The weakened portion 716 can extend longitudinally along any portion of the expandable sheath 700.

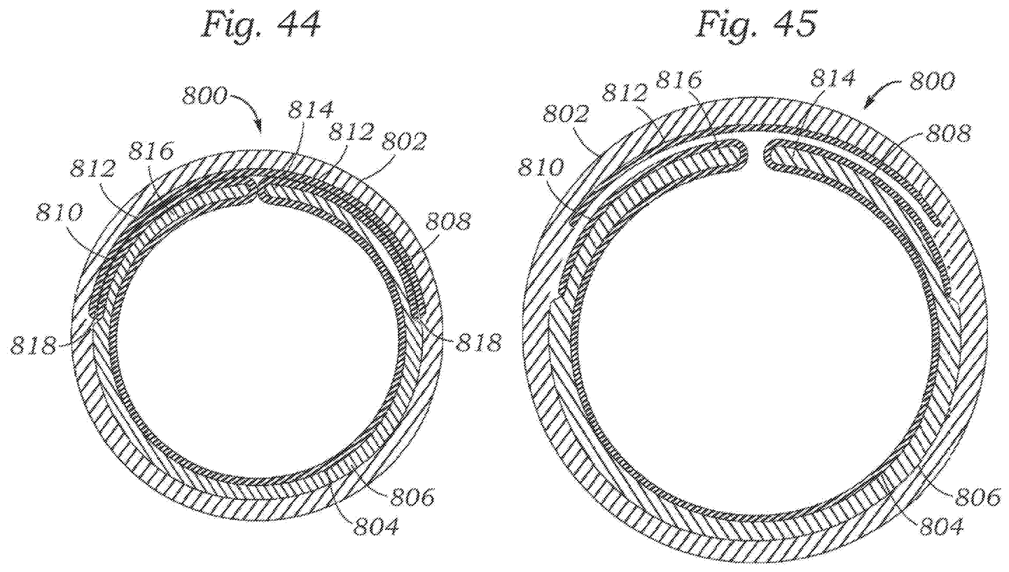

FIGS. 44 and 45 show another aspect of an expandable sheath 800 having an initial diameter in a resting configuration (FIG. 44) and a larger expanded diameter in an expanded configuration (FIG. 45). The sheath 800 can include an elongated tube, as disclosed above, 802, an inner layer 804, and an outer layer 806. Inner layer 804 can include first and second folded portions 808, 810. The folded portions 808, 810 can be arranged such that they fold away from one another in opposite directions around the circumference of the sheath 800. For example, folded portion 808 can be folded to the right in the view of FIG. 44 and folded portion 810 can be folded to the left such that they do not overlap one another but share a common segment 812, which is part of both folded portions 808, 810. In contrast to previous aspects, the outer layer 806 does not include an overlapping portion in this aspect but rather has first and second underlaying portions 814, 816, which underlie the first and second folded portions 808, 810, respectively. The inner layer 804 can extend through a gap between the ends of the adjacent underlaying portions 814, 816 (e.g., between a first end and a second end of discontinuous outer layer 806).

Each folded portion 808, 810 can include a weakened portion 818, such as a slit, score line, and/or perforation. Weakened portion 818 can allow the expandable sheath 800 to expand easily without a high radial force. As the sheath 800 expands, segment 812 along the top of the folded portions 808, 810 of inner layer 804 can be configured to split apart from the rest of the folded portions 808, 810 and the first and second underlaying portions 814, 816 can move away from one another so as to create an enlarged lumen within the inner layer 804. Weakened portions 818 can allow for segment 812 to easily split apart from the inner layer 804 as the sheath 800 expands.

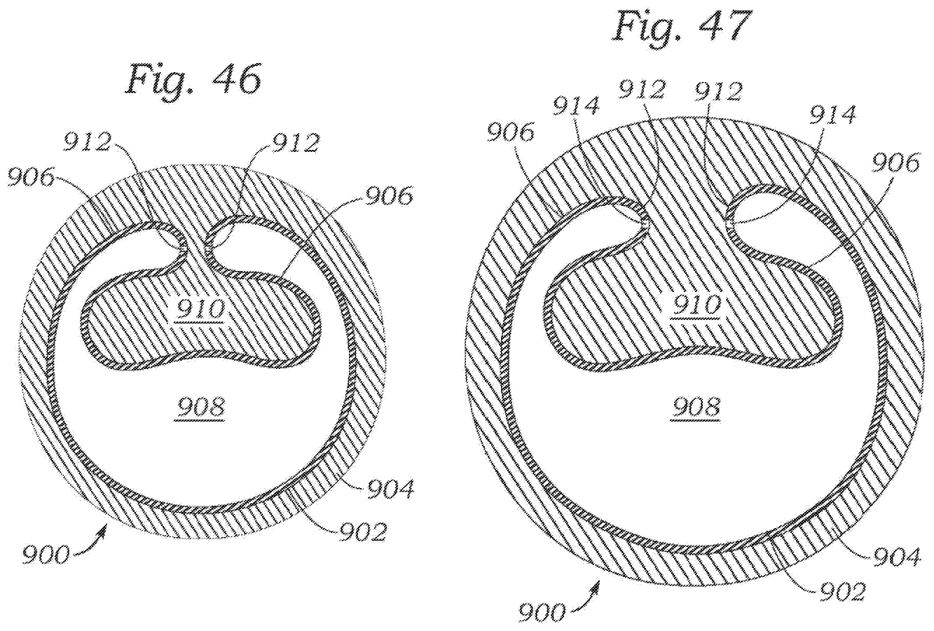

FIGS. 46-47 show another aspect of an expandable sheath 900. Sheath 900 can be provided with an inner layer 902 and an elongated tube 904 surrounding the inner layer 902. While not shown, sheath 900 can additionally include an intermediate layer positioned between the inner layer 902 and the elongated tube 904. If present, the intermediate layer can closely follow the contour of the inner layer 902.

Inner layer 902 can be shaped to include one or more folded portions 906 arranged to form a generally horseshoe-shaped lumen 908 that extends longitudinally through sheath 900 along the inner surface of the inner layer 902. The folded portions 906 can be arranged to form an area 910 positioned with the lumen 908 and radially inward from the elongated tube 904. In some aspects, the area 910 can include one or more voids (e.g., smaller lumens or openings extending through area 910). In some aspects, the area 910 can be filled with material (e.g., HDPE) reflowed from an intermediate layer while the sheath is being made. In some aspects, area 910 can be filled with material reflowed from the elongated tube 904 during the sheath manufacturing process.

The inner layer 902 can include one or more weakened portions 912, such as score lines, perforations, or slits. The weakened portions 912 can be configured to split apart, separate, or widen as the sheath expands from its initial resting configuration (FIG. 46) to an expanded configuration (FIG. 47) in the presence of a radial force. As the sheath 900 expands, material from the area 910 can cover any gaps 914 formed at the weakened portions 912, thereby keeping the lumen 908 substantially sealed.

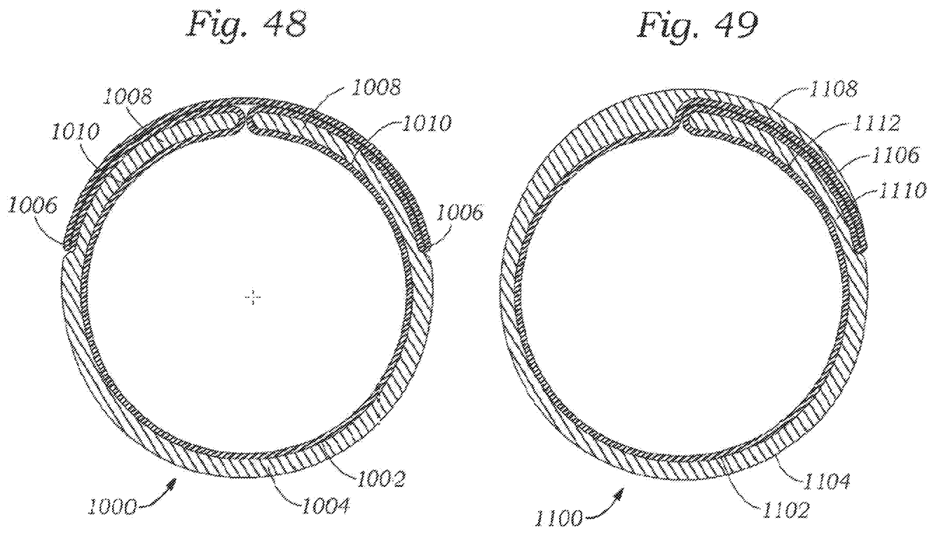

FIG. 48 shows another aspect of an expandable sheath 1000 having an inner layer 1002 and a discontinuous outer layer 1004. Sheath 1000 is similar to the sheath 800 of FIG. 44, except that sheath 1000 is shown without the disclosed herein elongated tube behaving as an outer jacket and further, the inner layer 1002 is continuous, without weakened portions at the folds 1006. As shown in FIG. 48, the inner layer 1002 can be configured to have one or more folds 1006 (e.g., two folds positioned on the outer surface of the outer layer 1004), with portions 1008 of the outer layer 1004 extending between the folds 1006 and the outer surface 1010 of the inner layer 1002 underlaying the folds 1006.

FIG. 49 shows yet another aspect of an expandable sheath 1100 having an inner layer 1102 and an outer layer 1104. The sheath 1100 is similar to the sheath 100 shown in FIG. 39 in that the inner layer 1102 can be continuous with a folded portion 1106, and the outer layer 1104 can be discontinuous with an overlapping portion 1108 overlapping at least a part of the folded portion 1106 and an underlaying portion 1110 underlaying at least a part of the folded portion 1106. The underlaying portion 1110 can thus be positioned between an outer surface 1112 of the lumen-forming portion of the inner layer 1102 and the folded portion 1106.

The inner layers 1002, 1102 of the sheaths 1000, 1100, respectively, of FIGS. 48-49 can be optimized to perform slightly differently than the inner layers of sheaths described above. For example, different materials can be used for the inner liner to increase the durability and softness of the seam (although such materials can also be used with the other aspects of expandable sheaths described above). For example, materials such as woven fabrics or braid filaments can be used. Such fabrics, filaments, or yarns can comprise, for example, PTFE, PET, PEEK, and/or nylon yarns or filaments. These materials can advantageously provide a soft and flexible layer that can be easily formed into the desired shapes or folded portions. Additionally, such materials can withstand high temperatures, as well as can possess high tensile strength and tear resistance. Nonetheless, these materials can also be elastic, experience minimal kinking, and provide soft distal edges for less traumatic insertion into a patient's vessels.

It is further understood that the aspects disclosed herein that describe the presence of the outer layer in addition to the inner layer and the elongated tube (the outer jacket) are exemplary. Also described herein are aspects where this outer layer is substituted fully by an elongated tube comprising the disclosed herein composition. In such aspects, the elongated tube can have a structure and characteristics of the as disclosed herein outer layer. It is further understood that in such exemplary and unlimiting aspects, the additional elongated tube can be present and behave as disclosed herein outer jacket or a strain relief jacket.

The elongated tube of this current disclosure can be used as an outer jacket 140 in various sheath configurations, as shown in FIG. 60B and FIGS. 71-77. The sheath comprising the elongated tube (or as used interchangeably, “outer jacket”) of the current disclosure can also comprise one or more axial reinforcing members 145 that extend longitudinally along all or a portion of the outer jacket 140. The reinforcing members 145 can be disposed in the first polymer layer, in the second polymer layer, or between the first and second layers.

It is understood that the axial reinforcing members as used herein can also be used interchangeably with the term “at least one intermediate reinforcement layer.” In certain aspects, the at least one intermediate reinforcement layer can be presented as a strip. While in yet other aspects, the at least one intermediate reinforcement layer can be a thermally bondable layer. Yet, in still further aspects, this thermally bondable layer can be presented as a strip.

The reinforcing member 145 provides stiffness and prevents axial bunching of the outer jacket 140 during insertion into the patient's vasculature while not sacrificing the low radial expansion force of the outer jacket 140.

As illustrated in FIG. 35, the sheath 100 can include a tapered segment adjacent the flared end portion 114 at the proximal end of the sheath 100. Referred to as a strain relief section, the tapered segment and the flared end portion 114 help ease the transition between the smaller diameter portion of the sheath 100 and the housing 101. The thickness and/or composition of the outer jacket 140 can be adjusted to increase the Durometer and/or stiffness along the strain relief section. Because this portion of the sheath 100 is usually outside of the patient's body during the procedure, providing the outer jacket 140 with an increased Durometer and/or stiffness along the strain relief section helps to withstand the blood pressure that would otherwise cause the outer jacket 140 to “balloon up” with body fluid/blood. As a result, it allows for a sheath 100 having a relatively stiff proximal end at the point of introducing a delivery apparatus while still having a relatively soft distal tip at the point of entry into the patient's vessel.

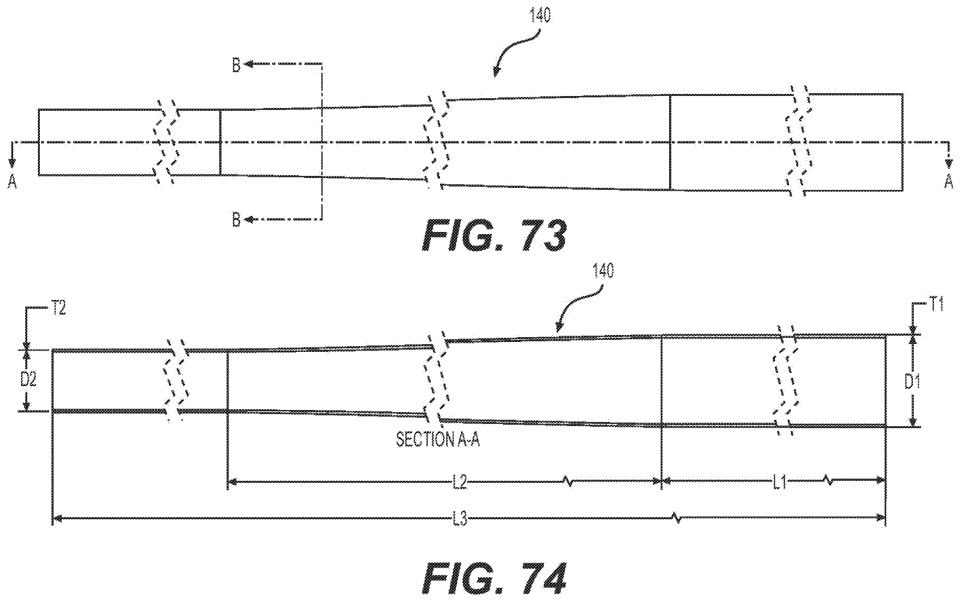

As disclosed herein, the elongated tube or an outer jacket can have the same diameter across the length of the sheath or can have varying diameters across the length of the sheath. FIG. 73 is an elevation view of the outer jacket 140 showing a tapered segment adjacent the flared end portion at the proximal end of the sheath. FIG. 74 is a cross section view of the outer jacket 140 taken along section A-A in FIG. 73. As described above, the tapered portion is referred to as a strain relief section, and the tapered segment and the flared proximal end help ease the transition between the smaller diameter portion of the sheath 100 and the housing 101. The length of the proximal end (L1) can range from 1.600 inches to 2.400 inches. In some aspects, the length of the proximal end is about 2.000 inches. The length of the tapered segment (L2) can range from 2.000 inches to 3.000 inches. In some aspects, the length of the tapered segment (L2) is about 2.500 inches. The overall length of the outer jacket 140/sheath 100 (L3) can range from 17.600 inches to 26.400 inches. In some aspects, the overall length of the outer jacket 140/sheath 100 (L3) is about 22.000 inches.

As provided in FIG. 73, the diameter of the outer jacket 140 at the proximal end is greater than the diameter of the outer jacket 140 at the distal end. This allows the outer jacket 140 to be slid over the inner and outer layers 108, 110 without having to be expanded. For example, the diameter of the outer jacket 140 at the proximal end can range from 0.264 inches to 0.396 inches. In some examples, the diameter of the outer jacket 140 at the proximal end is about 0.330 inches. The diameter of the outer jacket 140 at the distal end can range from 0.176 inches to 0.264 inches. In some examples, the diameter of the outer jacket at the distal end is about 0.220 inches.

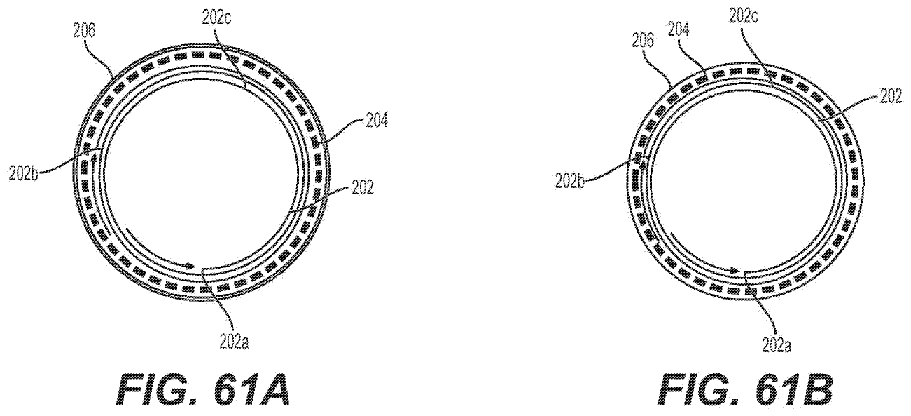

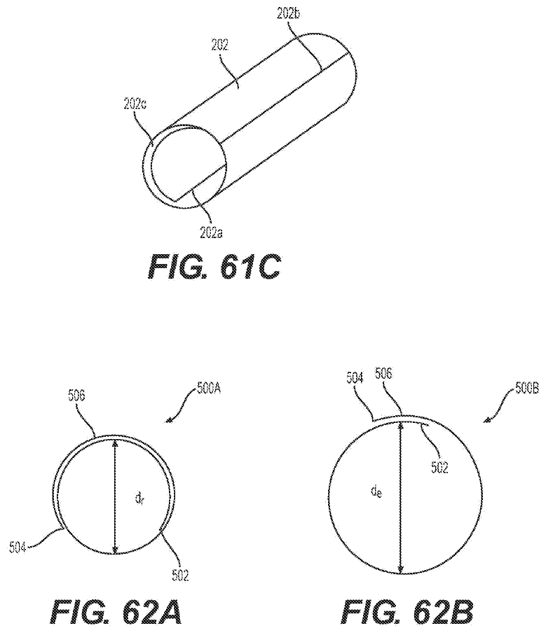

Additional aspects of the sheath comprising the disclosed herein elongated tube are shown in FIGS. 61A-C. In such aspects, the sheath further comprises a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and wherein the elongated tube is positioned such that the inner surface of the elongated tube overlies at least a portion of the outer surface of the inner liner (layer).

FIGS. 61A and 61B show section views of aspects of two exemplary sheaths disclosed herein for use with a delivery apparatus such as that shown in FIG. 1. FIG. 61C shows a perspective view of one aspect of an inner liner 202 for use with the disclosed sheath. As shown in FIGS. 61A-C, in some aspects, the disclosed sheath comprises an inner liner 202 wound in a spiral configuration such that at least a portion of the inner surface of the sheet overlays at least a portion of the outer surface of the sheet, forming an overlaying portion 202c, and wherein the first edge 202a of the sheet is slidable along at least a portion the inner surface of the sheet and the second edge 202b is slidable along at least a portion of the outer surface of the sheet. Sheath, as shown in FIG. 61A and FIG. 61B can further include a braid 204 and an elongated tube, as disclosed herein, 206. The braid 204 can be positioned between the inner liner and the elongated tube, for example, and as shown in FIG. 61A, the braid 204 that is not embedded into the elongated tube 206. While in the other aspect, and as shown in FIG. 61B, the braid 204 can be at least partially embedded into the layer of the elongated tube 206. In yet further aspects, the braid 204 and the elongated tube 206 form an outer layer of the sheath.

In still further aspects, the sheath, as disclosed herein, does not comprise the braid. In such exemplary and unlimiting aspects, the elongated tube 206 alone, without the braid, forms the outer layer of the sheath.

In still further exemplary and unlimiting aspects, an additional outer layer can be optionally positioned in between the inner liner and the elongated tube.

The inner liner 202 defines a lumen 201 through which a delivery apparatus can travel into a patient's vessel in order to deliver, remove, repair, and/or replace a prosthetic device. The disclosed sheath can also be useful for other types of minimally invasive surgery, such as any surgery requiring introduction of an apparatus into a subject's vessel. For example, the disclosed sheath also can be used to introduce other types of delivery apparatus for placing various types of intraluminal devices (e.g., stents, stented grafts, etc.) into many types of vascular and non-vascular body lumens (e.g., veins, arteries, esophagus, ducts of the biliary tree, intestine, urethra, fallopian tube, other endocrine or exocrine ducts, etc.). In still further exemplary aspects, the sheath can contract to the predetermined rest diameter dᵣ after passage of the medical device through the lumen.

In still further aspects, the sheet used to make the inner liner 202 can comprise a high-density polyethylene, polypropylene, polyamide, fluoropolymer, copolymers thereof, or blends thereof. In still further aspects, the sheet can comprise one or more layers. In some aspects, if one or more layers are present, each layer can comprise the same or different polymer. In still further aspects, the sheet can have a predetermined thickness, wherein the predetermined thickness can be defined by one of ordinary skill in the art depending on the specific application. In certain aspects, the predetermined thickness of the inner liner can be from about 0.002 inches to about 0.0025 inches, including exemplary values of about 0.003, about 0.004, about 0.005, about 0.006, about 0.007, about 0.008, about 0.009, about 0.01, about 0.015, and about 0.02 inches. It is further understood that the predetermined thickness of the sheet forming the inner liner 202 can be varied depending on the desired amount of radial expansion, as well as the strength required.

In still further aspects, the internal surface of the sheet can be at least partially ribbed. In yet further aspects, the sheet can also be lubricious. In some exemplary aspects, the sheet that forms the inner liner can have a coefficient of friction less than about 0.5, less than about 0.4, less than about 0.3, less than about 0.2, less than about 0.1, or less than about 0.05, or even less than about 0.01. It is further understood that the sheet can have a coefficient of friction having any value between any two foregoing values. Such a liner can facilitate the passage of a delivery apparatus through the lumen 201 of the disclosed sheath. In some further exemplary aspects, materials that can be used to form suitable lubricious inner liners include materials that can reduce the coefficient of friction of the inner liner 202, such as PTFE, polyethylene, polyvinylidene fluoride, and combinations thereof. Suitable materials for a lubricious liner also include other materials desirably having a coefficient of friction of about 0.1 or less, of about 0.09 or less, about 0.08 or less, about 0.07 or less, about 0.05 or less, about 0.04 or less, about 0.03 or less, about 0.02 or less, or about 0.01 or less.

In yet further aspects, the elongated tube can have any predetermined thickness. It is understood that the predetermined thickness of the elongated tube can be dependent on the specific application of the sheath. For example, and without limitation, the thicknesses of the inner liner 202, the elongated tube 206, and the braid 204 can also be varied depending on the particular application of the disclosed sheath. In some aspects, the thickness of the inner liner 202 ranges from about 0.0005 inches to about 0.010 inches, including exemplary values of about 0.0006, about 0.0007, about 0.0008, about 0.0009, about 0.001, about 0.002, about 0.003, about 0.004, about 0.005, about 0.006, about 0.007, about 0.008, about 0.009 inches, and in one particular aspect, the thickness can be about 0.002 inches. In yet other aspects, a total thickness of the elongated tube 206 and the braid 204 can have a thickness of from about 0.002 inches to about 0.015 inches, including exemplary values of about 0.003, about 0.004, about 0.005, about 0.006, about 0.007, about 0.008, about 0.009, and about 0.01 inches.

It is understood that the inner liner can have any shape or configuration depending on the desired application and the size of the delivery apparatus and prosthetic device. It is further understood that the inner liner is not limited to a specific shape or configuration. In certain aspects, the sheath disclosed herein is defined by the rest diameter dᵣ, and the outer diameter dₒ. As disclosed herein, the rest diameter dᵣ is defined by the inner liner, while the outer diameter can be defined by the inner liner and the outer layer, wherein the outer layer comprises the braid and the elongated tube.

The resting diameter dᵣ of the inner liner 202 can vary depending on the application and size of the delivery apparatus and prosthetic device. It is understood that in some aspects, the rest diameter dᵣ is substantially uniform along the longitudinal axis of the lumen without changing from the proximal end to the distal end. In yet other aspects, the rest diameter dᵣ can vary along the longitudinal axis of the lumen. In certain aspects, the rest diameter dᵣ at the proximal end is larger than the rest diameter dᵣat the distal end. In yet further aspects, where the outer layer comprising the braid and the elongated tube conforms to the shape of the inner liner, the outer diameter dₒ (not shown) comprises the overall diameter of the inner liner and the outer layer. In such aspects, the outer diameter dₒ is defined by the specific application of the sheath.

Similar to the rest diameter dᵣ, the outer diameter dₒ of the unexpended sheath disclosed herein can be substantially uniform (constant) along the longitudinal axis of the lumen without changing from the proximal end to the distal end (not shown). In alternative aspects, the original unexpanded outer diameter dₒ of the disclosed sheath, similarly to the rest diameter dᵣ, can decrease from the proximal end to the distal end. In some aspects, and similarly to the rest diameter dᵣ, the original unexpanded outer diameter can decrease along a gradient, from the proximal end to the distal end; or it can incrementally step down along the length of the sheath having the largest original unexpanded outer diameter is near dₒ the proximal end, and the smallest original unexpanded outer diameter dₒ is near the distal end.

In some aspects, the rest diameter dᵣ can range from about 0.005 inches to about 0.400 inches, including exemplary values of about 0.01 about 0.02, about 0.03, about 0.04, about 0.05, about 0.06, about 0.07, about 0.08, about 0.09, about 0.1, about 0.2, and about 0.3 inches. As described above, in certain aspects, the sheath can comprise the inner liner having various dᵣ. In such aspects, the dᵣ can have any value between any two foregoing values and can depend on the specific application and the size and shape of the delivery apparatus and prosthetic device. Different sheaths can be provided with different expanded and unexpanded rest diameter dᵣ and outer diameter dₒ, depending on the size requirements of the delivery apparatus for various applications. Additionally, some aspects can provide more or less expansion depending on the particular design parameters, the materials, and/or configurations used.

As disclosed herein, the outer layer comprises a braid 204 and the elongated tube 206 having a predetermined thickness and having an inner surface and outer surface (as shown in FIGS. 61A and 61B). In certain aspects, the braid can be an expandable braid. In yet further aspects, the braid can comprise at least one filament comprising stainless steel, nitinol, a polymer material, or a composite material. In certain unlimiting aspects, the braid comprises filaments comprising Nitinol and/or other shape memory alloys. In yet other unlimiting aspects, the braid can have filaments comprising polyester or nylon. In yet some other exemplary aspects, the braid can comprise filaments comprising spectra fiber, polyethylene fiber, aramid fiber, or combinations thereof. Again, as described above, the braid 204 is optional, and aspects without the braid are also disclosed.

It is understood that the braid can have any configurations known in the art. In certain aspects, the braid 204 is generally a thin, hollow, substantially cylindrical tube comprising an arrangement, pattern, structure, or configuration of filaments or struts, however, other geometries can also be used. Suitable filaments can be round, having a diameter less than about 0.015,″ less than about 0.01″, less than about 0.008″, less than about 0.005″, less than about 0.002″, less than about 0.001″, less than about 0.0008″, or less than about 0.0005″. In yet other aspects, suitable filaments can be round and having a diameter ranging from about 0.0005″ inches thick to about 0.015″ thick, including exemplary values of about 0.0006″, about 0.0007″, about 0.0008″, about 0.0009″, about 0.001″, about 0.002″, about 0.003″, about 0.004″, about 0.005″, about 0.006″, about 0.007″, about 0.008″, about 0.009″, about 0.01″, about 0.012″, about 0.013″, and about 0.014″. In yet other aspects, the suitable filaments can be flat filaments having a height of less than about 0.006″, less than about 0.005″, less than about 0.004″, less than about 0.003″, less than about 0.001″, less than about 0.0009″, less than about 0.0008″, less than about 0.0007″, less than about 0.0006″, and about 0.0005″. In yet other aspects, the flat filaments can have a width from greater than about 0.003″ to about 0.015″, including exemplary values of about 0.004″, about 0.005″, about 0.006″, about 0.007″, about 0.008″, about 0.009″, about 0.01″, about 0.012″, about 0.013″, and about 0.014″. However, other geometries and sizes are also suitable for certain aspects.

In yet further aspects, the braid can have a per-inch crosses (PIC) count of less than 50, less than 40, less than 30, less than 20, or less than 10. In yet other aspects, the braid can have a PIC count from 10 to 2, including exemplary values of 9, 8, 7, 6, 5, 4, and 3. In still further aspects, the PIC can vary along the longitudinal axis of the lumen. In yet other aspects, the braid pattern can vary along the longitudinal axis of the lumen. In the aspects where the braid comprises filament that is nitinol, the nitinol is heat-set at the expanded diameter dₑ. In yet further aspects, where the filament comprises stainless steel or nitinol, the filament is configured to be atraumatic, at least at the distal end of the sheath. FIGS. 9-23 illustrate partial elevation views of various structures for the braid 28. It is understood that the structure of the braid 28 can vary from section to section, changing along the length of the sheath. It is further understood that the structures shown in FIGS. 9-23 are not necessarily drawn to scale and show just exemplary and unlimiting aspects. It is further understood that the braid is configured to provide the torquability of the sheath during the insertion of the prosthetic device. Again, it is understood that the presence of the braid in these aspects is optional, and an aspect having a similar configuration without the presence of the braid is also disclosed.

It is understood that the elongated tube 206, as shown in FIGS. 61A and 61B can comprise any composition and exhibit any of the characteristics disclosed herein.

Alternative aspects of a sheath for introducing a prosthetic device are also described. For example, FIGS. 62A-62B illustrate a section view of the inner liners 500A and 500B of the disclosed sheath in unexpended and expended configurations (FIGS. 62A and 62B, respectively). Upon introduction of the prosthetic device into the inner liner, the first edge 502 and the second edge 504 slid along and expand the inner liner from the rest diameter dᵣ to the expanded diameter dₑ, thereby shortening the overlaying portion 506 of the inner liner. It is understood that the expanded diameter dₑ is configured to accommodate the medical device passing through the lumen. In yet further aspects, the sheath contracts to the predetermined rest diameter dᵣ after passage of the medical device through the lumen.

In certain aspects, an amount of a first lubricant is disposed between at least a portion of the inner liner and at least a portion of the outer layer that comprises the braid and the disclosed elongated tube. In yet other aspects, an amount of a second lubricant is disposed between at least a portion of the overlying portion of the sheet and at least a portion of the sliding portions of the sheet. It is understood that the first lubricant and the second lubricant can be the same or different. In certain and unlimiting aspects, the first and/or second lubricants can comprise Christo Lube supplied by ECL or MED10/6670 supplied by Nusil. In still further aspects, it is understood that the amount of the first and/or second lubricant can be easily determined by one of ordinary skill in the art.

In still further aspects, the outer surface of the elongated tube defines at least a portion of the outer surface of the outer layer. In yet other aspects, at least a portion of the inner surface of the elongated tube is at least partially bonded to at least a portion of the outer surface of the sheet of the inner liner. It is understood that the outer layer of the disclosed sheath is configured to provide hemostasis and prevent bleeding of the patient during the procedure.

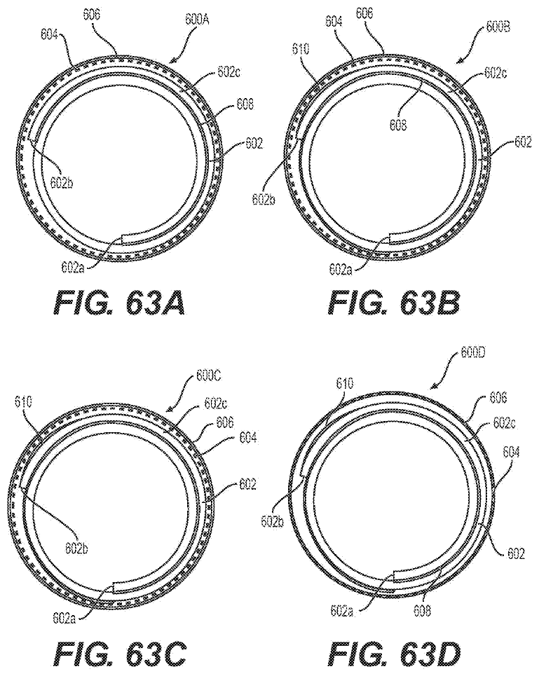

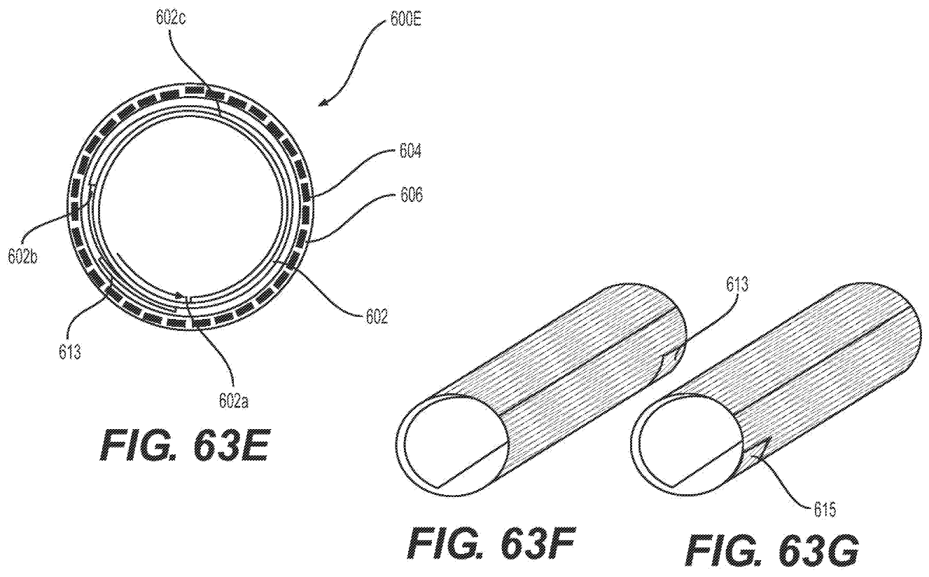

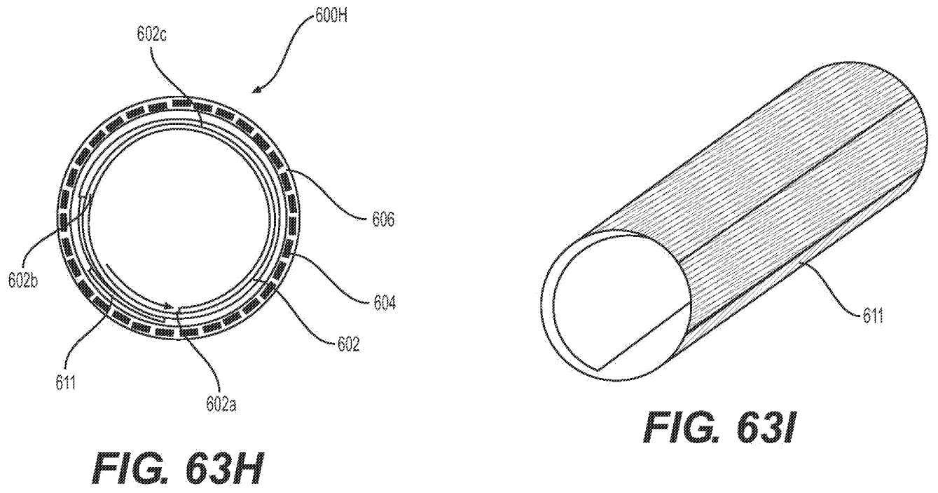

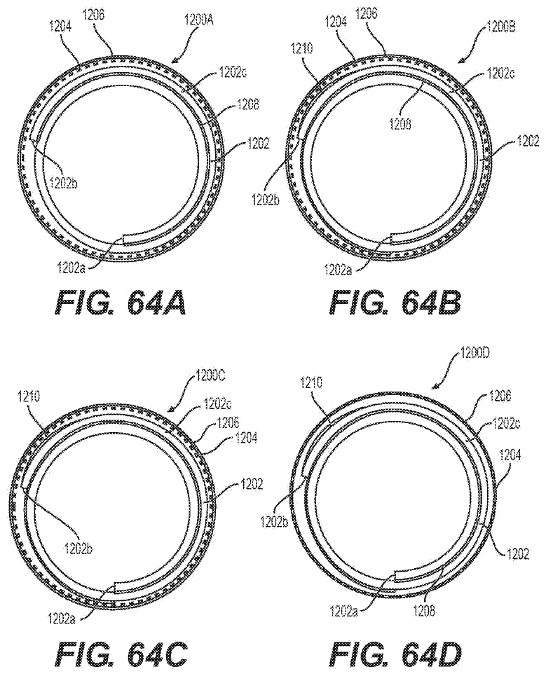

FIGS. 63A-63I show other alternative aspects of a sheath for introducing a prosthetic device. FIG. 63A shows the sheath 600A comprising the inner liner 602 having the first edge 602a and the second edge 602b, and the overlaying portion 602c, where the inner and outer surfaces of the inner liner overlay each other. The sheath 600A further comprises an amount of the second lubricant 608, as disclosed herein, that is disposed between the sliding and overlaying portions of the inner sheath. The sheath further comprises the braid 604 and the elongated tube 606. In this exemplary aspect, the braid 604 is not embedded into the elongated tube 606. FIG. 63B depicts an alternative aspect of the sheath 600B where an amount of the first lubricant 610 is applied between the inner liner and the outer layer comprising the braid 604 and the elongated tube 606. An additional aspect of the sheath 600C is shown in FIG. 63C. In this aspect, the sheath 600C comprises the inner liner 602, having the first edge 602a and the second edge 602b, and the overlaying portion 602c, where the inner and outer surfaces of the inner liner overlay each other. The sheath further comprises the braid 604 and the elongated tube 606, that together can form the outer layer of the sheath. The sheath 600C further comprises an amount of the first lubricant 610, as disclosed herein, that is disposed between the outer layer and the inner liner of the inner sheath. In this exemplary aspect, the braid 604 is not embedded into the elongated tube 606. In the exemplary aspect shown in FIG. 63D, the exemplary sheath 600D, comprises the braid 604 embedded within the elongated tube 606.

In still further aspects, the sheath of the instant disclosure can comprise a hemostasis valve inside the lumen of the sheath, at or near the proximal end of the sheath (not shown). Additionally, the exemplary sheaths disclosed herein can comprise a soft tip at the distal end of the sheath (not shown). Such a soft tip can be provided with a lower hardness than the other portions of the sheath. In some aspects, the soft tip can have a Shore hardness from about 25 D to about 40 D, including exemplary values of about 26 D, about 27 D, about 28 D, about 29 D, about 30 D, about 31 D, about 32 D, about 33 D, about 34 D, about 35 D, about 36 D, about 37 D, about 38 D, and about 39 D. In yet other aspects, the soft tip can have a Shore hardness from about 25 A to about 40 A, including exemplary values of about 26 A, about 27 A, about 28 A, about 29 A, about 30 A, about 31 A, about 32 A, about 33 A, about 34 A, about 35 A, about 36 A, about 37 A, about 38 A, and about 39 A.

In certain aspects, the elongated tube and the inner liner can be bonded together or otherwise physically associated with one another. It is understood that the amount of adhesion between the inner liner 602 and the outer polymer layer that comprises braid 604 and the elongated tube 606 can be variable over the surfaces of the layers. The bonding between the layers can be created by, for example, thermal bonding. In certain aspects, the bonding can be facilitated by the presence of an additional portion of the elastomeric polymer. For example, in certain aspects, the sheath, as described herein and as shown in FIGS. 63H-I can further comprise a first strip 611 of the elastomeric polymer disposed along at least a portion of the longitudinal axis of the lumen between at least a portion of the outer surface of the sheet that does not comprise the overlaying portion 602c of the sheet and the inner surface of the elongated tube. In such aspects, the bonding between the elongated tube and the inner liner can be facilitated by the first strip of the elastomeric polymer. In yet other aspects, the sheath can optionally, if desired, further comprise a second strip 613 (FIGS. 63E-F) of the elastomeric polymer disposed between at least a portion of the outer surface of the sheet at the proximal end of the sheath and the inner surface of the elongated tube. In still further aspects, the sheath can further comprise a third strip 615 of the elastomeric polymer disposed between at least a portion of the outer surface of the sheet at the distal end of the sheath and the inner surface of the elongated tube (FIG. 63G). Again, in such aspects, the bonding between the elongated tube and the inner liner can be facilitated by the second and/or third strips of the elastomeric polymer.

Applications can utilize a sheath of the present disclosure with the rest diameter dᵣ of the lumen formed by the inner liner 602 that is expandable to an expanded diameter dₑ of from about 3 Fr to about 26 Fr, including exemplary values of about 5 Fr, about 8 Fr, about 10 Fr, about 12 Fr, about 15 Fr, about 18 Fr, about 20 Fr, about 22 Fr, about 25 Fr. The expanded diameter can vary slightly along the length of the disclosed sheath. For example, the expanded outer diameter at the proximal end of the sheath can range from about 3 Fr to about 28 Fr, including exemplary values of about 5 Fr, about 8 Fr, about 10 Fr, about 12 Fr, about 15 Fr, about 18 Fr, about 20 Fr, about 22 Fr, about 25 Fr, while the expanded outer diameter at the distal end of the sheath can range from about 3 Fr to about 25 Fr, including exemplary values of about 8 Fr, about 10 Fr, about 12 Fr, about 15 Fr, about 18 Fr, about 20 Fr, and about 22 Fr. Aspects of the disclosed sheath can expand to an expanded outer diameter that is from about 10% greater than the original unexpanded outer diameter to about 100% greater than the original unexpanded outer diameter, including exemplary values of about 15% greater, about 20% greater, about 25% greater, about 30% greater, about 35% greater, about 40% greater, about 45% greater, about 50% greater, about 55% greater, about 60% greater, about 65% greater, about 70% greater, about 75% greater, about 80% greater, about 85% greater, about 90% greater, and about 95% greater than the original unexpanded outer diameter.

It is understood, and as described above, the disclosed sheath can expand from its rest position. The expansion of the disclosed sheath can result in an expansion of the rest diameter dᵣ of from about 10% or less to about 430% or more. In certain aspects, expansion of the sheath can result in expansion of the rest diameter dᵣ to about 10% or less, to about 9% or less, to about 8% or less, to about 7% or less, to about 6% or less, to about 5% or less, to about 4% or less, to about 3% or less, to about 2% or less, to about 1% or less. In yet other aspects, expansion of the disclosed sheath can result in expansion of the rest diameter dᵣ to about 10% or more, about 20% or more, about 30% or more, about 40% or more, about 50% or more, about 60% or more, about 70% or more, about 80% or more, about 90% or more, about 100% or more, about 125% or more, about 150% or more, about 175% or more, about 200% or more, about 225% or more, or about 250% or more.

As with previously disclosed aspects, the aspects illustrated in FIGS. 63A-63D can be applied to sheaths having a wide variety of rest diameters dᵣ and outer diameter dₒ. In some aspects, the outer diameter dₒ of the sheath gradually decreases from the proximal end of the sheath to the distal end of the sheath. For example, in one aspect, the outer diameter dₒ can gradually decrease from about 26 Fr at the proximal end to about 18 Fr at the distal end. The diameter dₒ of the sheath can transition gradually across substantially the entire length of the sheath. In other aspects, the transition or reduction of the diameter of the sheath can occur only along a portion of the length of the sheath. For example, the transition can occur along a length from the proximal end to the distal end, where the length can range from about 0.5 inches to about the entire length of the sheath, including any values between any two foregoing values. In yet further aspects, the dₒ is minimal and constant along the section of the sheath that passes through the vasculature. In such aspects, the tapered section is about 4″ or less at the proximal side of the sheath.

In some aspects, the braid and/or the elongated tube can comprise the same material or combination of materials along the entire length. In alternative aspects, the material composition of each layer can change along the length of the sheath. For example, the outer layer comprising both the braid and the elongated tube can be provided with one or more segments, where the composition changes from segment to segment. For example, in one segment, the braid can comprise nitinol having a different PIC count than another segment. In yet another exemplary aspect, the elongated tube in one segment can be different from the layer of the elastomeric material in another segment. In still further exemplary aspects, one segment of the sheath can comprise the braid embedded within the elongated tube, while another segment can comprise the braid that is not embedded within the elongated tube. It is understood that the exemplary sheath disclosed herein is not limiting. In certain exemplary aspects, the sheath can comprise an n number of segments, wherein each segment can be the same or different. In still further exemplary aspects, the Durometer rating of the composition of the outer layer can also change along the length of the sheath such that segments near the proximal end comprise a stiffer material or combination of materials, while segments near the distal end comprise a softer material or combination of materials. This can allow for a sheath having a relatively stiff proximal end at the point of introducing a delivery apparatus while still having a relatively soft distal tip at the point of entry into the patient's vessel.

Additionally, some aspects of the sheath, as disclosed herein, can include an exterior hydrophilic coating on the outer surface of the elongated tube. Such a hydrophilic coating can facilitate insertion of the sheath into a patient's vessel. Examples of suitable hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands), as well as other hydrophilic coatings (e.g., PTFE, polyethylene, polyvinylidene fluoride), are also suitable for use with the sheath. This exemplary sheath, similarly to other sheaths disclosed herein in some aspects, can comprise a soft tip portion comprising, in some aspects, low-density polyethylene (LDPE) and can be configured to minimize trauma or damage to the patient's vessels as the sheath is navigated through the vasculature. Any materials described herein for a soft tip can be used to form the soft tip of this exemplary sheath.

In certain aspects, the exemplary sheath, as described herein, can also be generally represented, as shown in FIG. 35. In such exemplary aspects, the described herein sheath can have additional components, as shown in FIGS. 35 and 36. It is, however, understood that the sheath disclosed herein does not have to comprise the components shown in FIGS. 35 and 36 and can be adapted to any other applications. In such exemplary aspects, it can be adapted to passing any medical devices that require an introducer sheath.

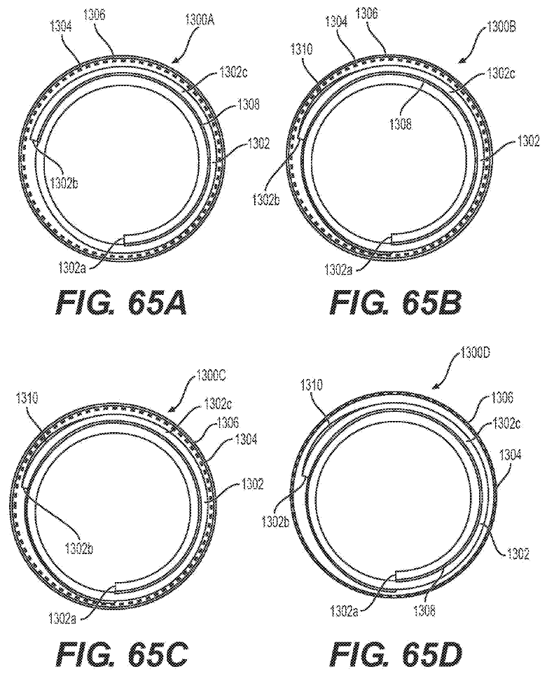

Some exemplary aspects of these additional sheaths are also shown in FIGS. 64A-64B and 65A-65B. FIGS. 64A-64B, for example, show a cross-section view of the exemplary sheath taken near the distal end. FIG. 64A shows the sheath 1200A comprising the inner liner 1202 having the first edge 1202a and the second edge 1202b, and the overlaying portion 1202c, where the inner and outer surfaces of the inner liner overlay each other. The sheath 1200A further comprises an amount of the second lubricant 1208 as disclosed herein that is disposed between the sliding and overlaying portions of the inner sheath. The sheath further comprises the braid 1204 and the elongated tube 1206. In this exemplary aspect, the braid 1204 is not embedded into the layer of the elongated tube 1206. FIG. 64B depicts an alternative aspect of the sheath 1200B where an amount of the first lubricant 1210 is applied between the inner liner and the outer layer comprising the braid 1204 and the elongated tube 1206. An additional aspect of the sheath 1200C is shown in FIG. 64C. In this aspect, the sheath 1200C comprises the inner liner 1202, having the first edge 1202a and the second edge 1202b, and the overlaying portion 1202c, where the inner and outer surfaces of the inner liner overlay each other. The sheath further comprises the braid 1204 and the elongated tube 1206 that together form the outer layer of the sheath. The sheath 1200C further comprises an amount of the first lubricant 1210, as disclosed herein, that is disposed between the outer layer and the inner liner of the inner sheath. In this exemplary aspect, the braid 1204 is not embedded into the elongated tube 1206. In the exemplary aspect shown in FIG. 64D, the exemplary sheath 1200D, comprises the braid 1204 embedded within the elongated tube 1206.

FIGS. 65A-D show a section view of a proximal section of the sheath. FIG. 65A shows the sheath 1300A comprising the inner liner 1302 having the first edge 1302a and the second edge 1302b, and the overlaying portion 1302c, where the inner and outer surfaces of the inner liner overlay each other. The sheath 1300A further comprises an amount of the second lubricant 1308 as disclosed herein that is disposed between the sliding and overlaying portions of the inner sheath. The sheath further comprises the braid 1304 and the elongated tube 1306. In this exemplary aspect, the braid 1304 is not embedded into elongated tube 1306. FIG. 65B depicts an alternative aspect of the sheath 1300B, where an amount of the first lubricant 1310 is applied between the inner liner and the outer layer comprising the braid 1304 and the elongated tube 1306. An additional aspect of the sheath 1300C is shown in FIG. 65C. In this aspect, the sheath 1300C comprises the inner liner 1302, having the first edge 1302a and the second edge 1302b, and the overlaying portion 1302c, where the inner and outer surfaces of the inner liner overlay each other. The sheath further comprises the braid 1304 and the elongated tube 1306 that together form the outer layer of the sheath. The sheath 1300C further comprises an amount of the first lubricant 1310, as disclosed herein, that is disposed between the outer layer and the inner liner of the inner sheath. In this exemplary aspect, the braid 1304 is not embedded into the elongated tube 1306. In the exemplary aspect shown in FIG. 65D, the exemplary sheath 1300D, comprises the braid 1304 embedded within elongated tube 1306.

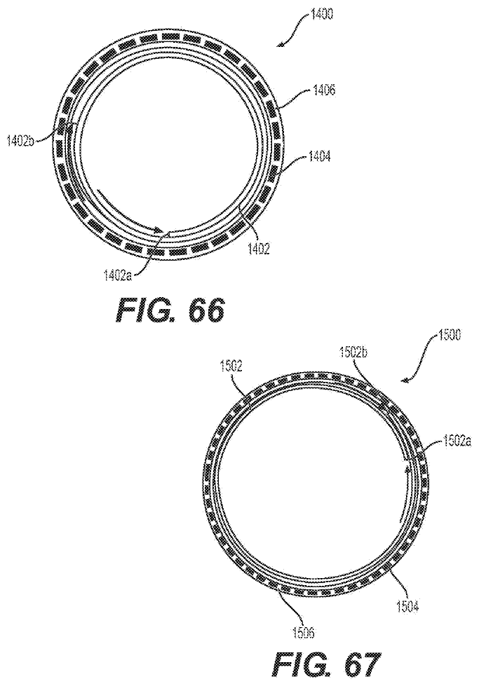

In yet further aspects, as shown in FIG. 66, the sheath 1400, whether with the braid embedded within the elongated tube (as shown in FIG. 66) or with the braid that is not embedded within the layer of the elastomeric polymer (not shown), is configured to expand from a resting configuration to an expanded configuration shown in FIG. 67. In such aspects, the first and the second edges (1502a and 1502b) of the inner liner slide such that a length of the overlaying portion shortens. In some exemplary aspects, this movement can be facilitated by the presence of the first and/or second lubricant, as disclosed above.

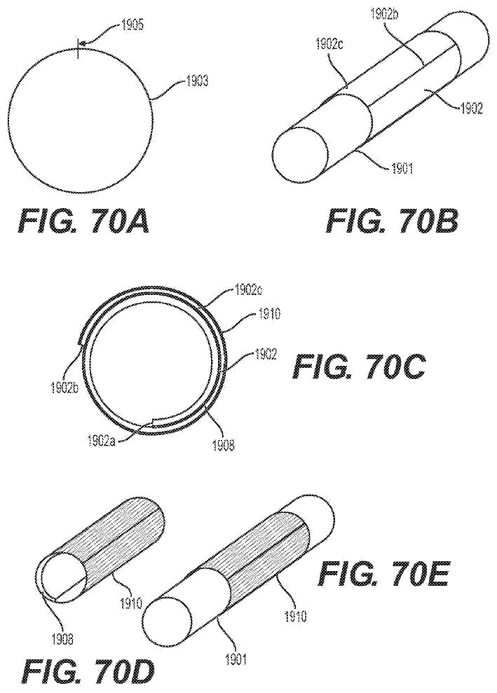

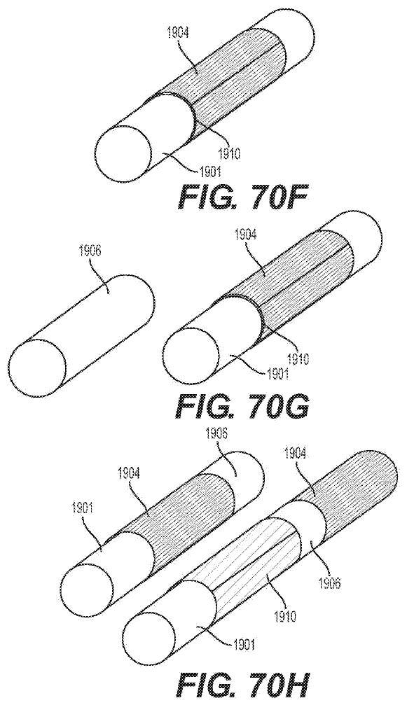

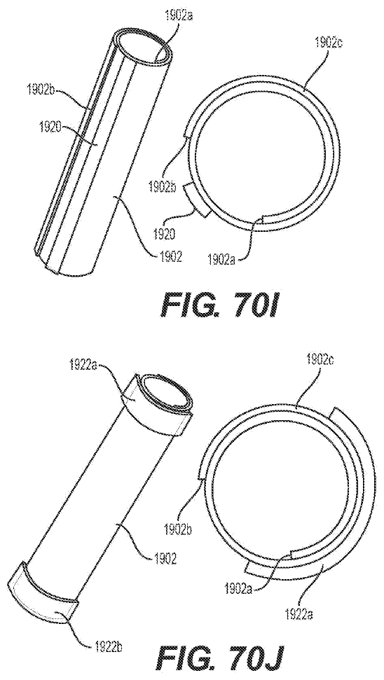

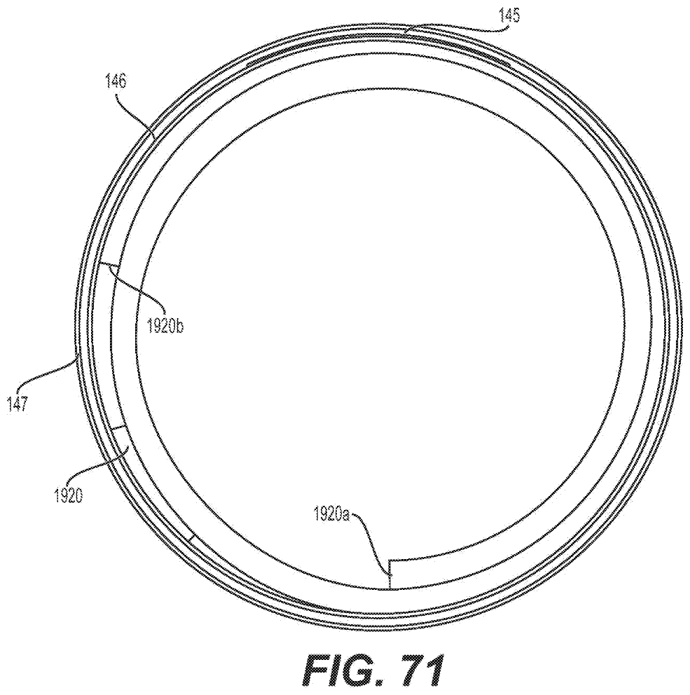

Now referring to FIGS. 71-72. As disclosed herein, the elongated tube can be used as an outer jacket. The outer jacket 140 disclosed herein can comprise at least two polymer layers 146 and 147. In still further aspects, the outer jacket disclosed herein can comprise at least one intermediate reinforcement layer/member 145 is disposed in the first polymer layer, in the second polymer layer, or between the first and second layers. Such an outer jacket can be disposed on an additional optional polymer strip 1920 as shown and further described as referenced to FIG.70.

FIGS. 73-77 illustrate an expandable outer jacket including longitudinally extending reinforcing members 145. The outer jacket 140 can be used with any of the expandable sheaths described herein. The reinforcing members 145 prevent axial bunching of the outer jacket 140 during insertion into the patient's vasculature while not sacrificing the low radial expansion force of the outer jacket 140. The reinforcing members 145 are typically constructed from a stiffer material (e.g., Pebax, polyurethane, nylon, flat wire) than the main body portion of the outer jacket 140. The resistance of the reinforcing members 145 to elongation and/or compression prevents bunching/crumpling of the outer jacket 140 during insertion while still allowing the outer jacket 140 to radially expand.

The wall thickness of the outer jacket can range from 0.0040 inches to 0.0066 inches. In some examples, the thickness of the outer jacket is about 0.0055 inches. The wall thickness of the outer jacket 140 can remain constant along the entire length of the outer jacket 140. However, in some examples, the thickness of the outer jacket 140 at the proximal end (T1) is greater than the thickness of the outer jacket 140 at the distal end (T2).

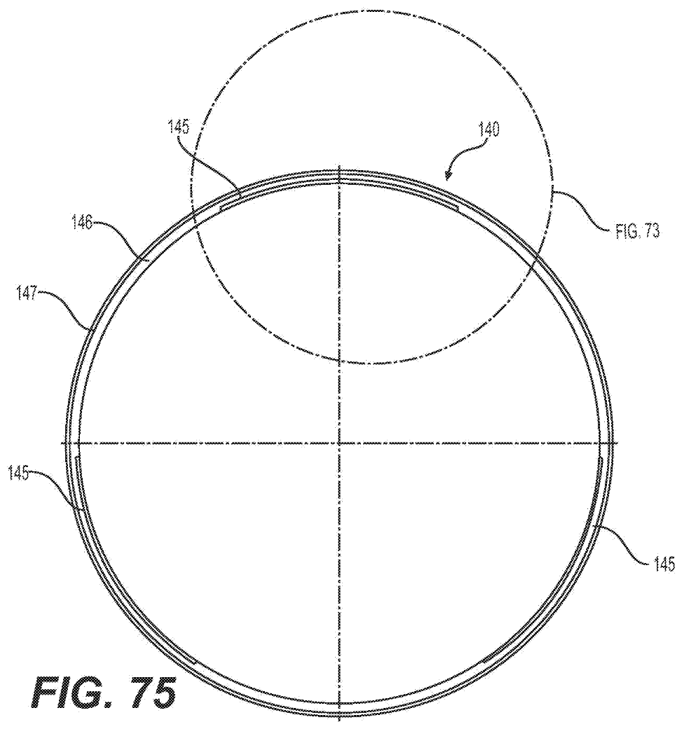

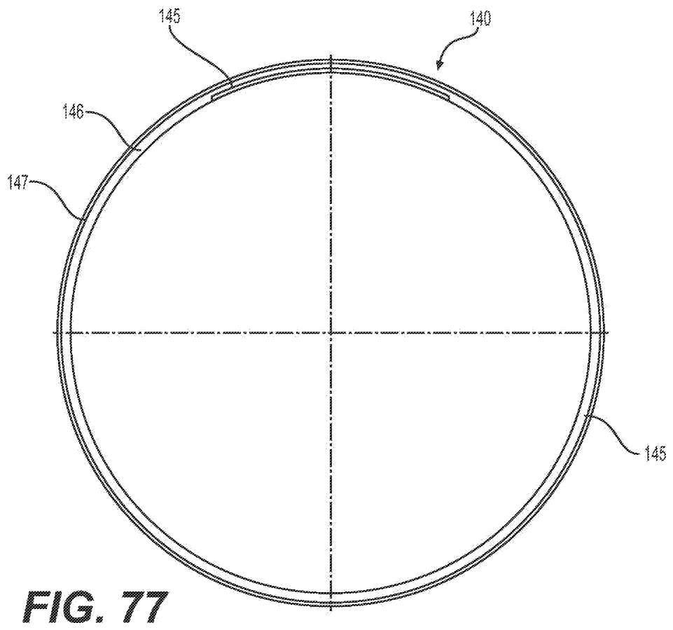

In still further aspects, the outer jacket 140 can comprise two or more reinforcing members 145. In such aspects, the two or more reinforcing members 145 can be disposed, as individual strips, disposed circumferentially in the first polymer layer, in the second polymer layer, or between the first and second layers at a predetermined distance from each other. FIG. 75 is a cross section view of the outer jacket 140 taken along section lines B-B in FIG. 73. As provided in FIG. 75, the outer jacket 140 includes three reinforcing members 145. In some examples, the outer jacket 140 includes only one reinforcing member 145 (FIG. 77). In other examples, the outer jacket includes up to eight reinforcing members 145. When more than one reinforcing member 145 is used, the reinforcing members are spaced evenly around the circumference of the outer jacket 140. As further illustrated in FIG. 75, the reinforcing member 145 can have a rectilinear shape (e.g., rectangular) in the cross section. However, any other regular or irregular shape is contemplated.

In further aspects, the reinforcing member 145 has a finite width that is smaller than the circumference of the outer jacket 140. The total combined width (w) of the reinforcing members 145 can range from 5% to 50% of the circumference of the outer jacket 140. In still further aspects, the total combined width of the strips is about 5%, about 15%, about 20%, about 25%, about 30%, about 35%, about 40%, about 45%, or about 50% of the circumference of the elongated tube.

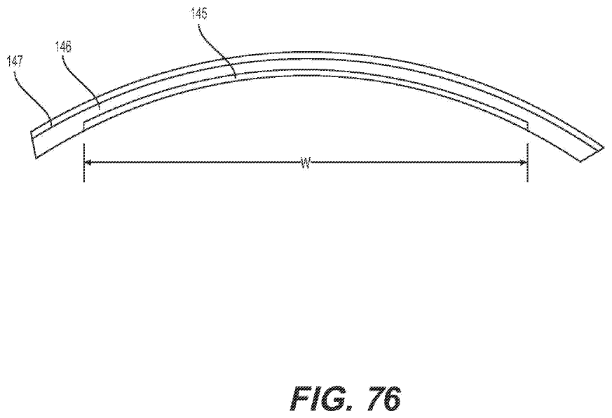

FIG. 76 includes a partial view of the outer jacket 140 of FIG. 75. As provided in FIG. 76, the circumferential width of the reinforcing members 145 can range from 0.010 inches to 0.150 inches. In some examples, the distal end of the outer jacket 140 has a diameter of 0.200 inches, and the circumferential width of the reinforcing members 145 can range from 0.010 inches to 0.150 inches. In some exemplary and unlimiting aspects, the diameter of the outer jacket at the distal end is about 0.200″, the reinforcing member can have a width between about 0.010″ to about 0.150″, including exemplary values of about 0.03″, about 0.035″, about 0.04″, about 0.045″, about 0.05″, about 0.055″, about 0.06″, about 0.065″, about 0.07″, about 0.075″, about 0.08″, about 0.085″, about 0.09″, about 0.095″, about 0.10″, about 0.105″, about 0.110″, about 0.115″, about 0.120″, about 0.125″, about 0.130″, about 0.135″, about 0.140″, and about 0.145″. It is understood that the widths shown above are exemplary, and if the distal outer diameter of the elongated sheath has a size different from 0.200″, the strip width can be adjusted in the same or a different ratio. In yet still further aspects, as described above, the width of the reinforcing member can be measured as a percentage of the elongated tubing circumference.

It is further understood that in the aspects where the reinforcing member 145 is present as one or more strips disposed circumferentially along the length of the outer jacket 140, the width of the reinforcing member 145 can be the same along the length, or it can vary along the length. In aspects where the reinforcing member 145 width varies along the length of the outer jacket 140, such a reinforcing member 145 can have any of the disclosed above width values.

In still further aspects, at least one reinforcing member 145 is configured to provide an axial reinforcement to the outer jacket 140 and, as a result, to the sheath where the outer jacket 140 can be used. In such exemplary aspects, the at least one reinforcing member 145 can be disposed along the length of the outer jacket 140 or along a portion of the length of the outer jacket 140. In some aspects, the portion of the length of the outer jacket 140 where the at least reinforcing member 145 is disposed at the distal end and/or proximal end of the outer jacket 140. In yet other aspects, the reinforcing member 145 can also be positioned anywhere along the length of the outer jacket 140.

As described above, and as illustrated in FIG. 75, the outer jacket 140 includes a two-layer construction, inner layer (first polymer layer) 146 and outer layer (second polymer layer) 147, where the outer layer provides abrasion resistance (for example, between the sheath and a calcific lesion) and better resistance to the hydrophilic coating process, and the inner layer is a more lubricious material (for example, to prevent sticking of the outer jacket against the outer layer of the sheath during expansion) the and provides higher pressure resistance or ballooning resistance and hemostasis. In some aspects, the inner layer 146 (first polymer layer) forms the inner surface of the outer jacket 140 and the outer layer 147 (second polymer layer) forms the outer surface of the outer jacket, the reinforcing members 145 are disposed between the outer surface of the inner layer (liner) 146 and the inner surface of the outer layer 147.

In some examples, the inner layer 146 can be composed of Pebax or polyurethane, having Shore 25 D to 35 D. In some examples, the inner layer 146 includes a PTFE powder, an optional inorganic filler, and an optional tackiness reducing additive to lower friction when outer layer 147 of the sheath expanding by sliding against the outer jacket 140. In some examples, the outer layer 147 of the outer jacket 140 is composed of polyurethane or polyurethane/Styrene Block Copolymer (SBC) having Shore A durometer lower than about 60, e.g., Neusoft 597-50A having Shore A hardness of about 55 A. In certain examples, the inner layer 146 is constructed from Polyether Block Amide, such as Pebax having a Shore D durometer of less than about 35.

As provided in FIGS. 75 and 76, the reinforcing members 145 are at least partially embedded in the inner layer 146. In some examples, the thickness of the reinforcing member 145 is less than the thickness of the inner layer 146. For example, as illustrated in FIG. 76, the reinforcing members 145 have a thickness ranging from 0.0005 inches to 0.0015 inches. In some examples, the reinforcing members 145 have a thickness of about 0.001 inches. In an example configuration, the reinforcing members 145 have a thickness of 0.001 inches, and the inner layer has a thickness of 0.00154 inches. In another example, not shown, the reinforcing member 145 has a thickness corresponding to the thickness of the inner layer 146. In a further example, the reinforcing member 145 has a thickness greater than the thickness of the inner layer 146. In some examples, the inner layer 146 and the reinforcing member 145 are co-extruded. Similarly, the inner layer 146, reinforcing member 145 and the outer layer 147 are co-extruded with the reinforcing member 145 positioned between the inner and outer layers 146, 147. In other examples, the inner layer 146 is provided over the reinforcing member 145, and the two components are bonded or fused together by at least one of heat or compression.

As described above, the reinforcing members 145 are constructed from a stiffer material than the main body portion of the outer jacket 140 (inner layer 146, outer layer 147) and also a material having a low coefficient of friction (e.g., high density polyethylene). In some examples, the reinforcing members 145 are constructed from a polymer compatible with the inner layer and outer layer, including, for example, high durometer Pebax or polyurethane. The reinforcing member 145 can also be constructed from a material having a Shore D durometer ranging from 45 D to 76 D.

Additional examples of the sheath that can be used with the disclosed herein elongated tube can be found in U.S. application No. 63/021,945, the content of which is incorporated herein in whole entirety.

The disclosed herein sheath can be configured such that it locally expands at a particular location corresponding to the location of the medical device along the length of the lumen and then locally contracts once the medical device has passed that particular location. Thus, a bulge may be visible, traveling longitudinally along the length of the sheath as a medical device is introduced through the sheath, representing continuous local expansion and contraction as the device travels the length of the sheath. In some aspects, each segment of the sheath can locally contract after removal of any radial outward (insertion) force such that it regains the original resting diameter of lumen dᵣ.

In some aspects, each segment of the sheath can locally contract after removal of any radial outward force such that it at least partially returns to the original resting diameter of lumen dᵣ.

### Methods

An example method of making the sheath is as follows. These steps are not meant to be limiting. The steps given can be reordered as needed. Other steps can be added, or in other examples, some steps may not be necessary.

Disclosed herein are the methods of making a sheath having a proximal end and a distal end and comprising: a) extruding a tubular body to form an elongated tube comprising a first polymer layer, wherein the first polymer layer comprises a first compound composition comprising from greater than 0 wt % to less than 100 wt % of a first polymer comprising a polyether block amide, a polyurethane, or a combination thereof; less than about 65 wt % of an inorganic filler based on a total weight of the first compound composition; and up to about 20 wt % of a solid lubricant filler based on a total weight of the first compound composition; b) disposing the elongated tube on the sheath such that the elongated tube forms an outer layer of the sheath, and wherein the elongated tube is positioned at at least the proximal end of the sheath and extending along at least a portion of a length of the sheath, wherein the elongated tube is configured to reversibly expand from an initial diameter do in an unexpended position to an expanded diameter dₑ in an expanded position upon passage of a medical device; and wherein the formed sheath exhibits at least a 10% reduction in an insertion force when compared with a substantially identical reference sheath that does not comprise the first polymer layer.

It is understood that any known in the art methods can be utilized to form the composition of the elongated tube. In certain aspects, the components that are present in the elongated tube are provided to form a compound. The compound is then mixed to form a substantially homogeneous mixture. Yet, in other aspects, the mixture is homogeneous. In still further aspects, the mixture is extruded to form an elongated tube having a first polymer layer. The formed first polymer layer can comprise any (and in any combination) of the compositions and characteristics disclosed above.

In yet further aspects, the methods also comprise steps of forming the elongated tube comprising two or more layers, as disclosed above. In such aspects, for example, when the elongated tube comprises any of the disclosed above first polymer and the second polymer layers, such layers can be coextruded to form the elongated tube as disclosed. Any of the known in the art extrusion devices can be used to obtain the desired elongated tube.

The methods disclosed herein produce any of the disclosed above elongated tubes.

In still further aspects, also disclosed are the methods of forming additional parts of the sheath. In certain aspects, the elongated tube can behave as an outer jacket of the sheath. While yet in other aspects, the elongated tube can form an outer layer of the sheath without an additional outer jacket being present.

For example, and without limitation, the inner layer 108, as shown in one of the exemplary sheaths, can be formed to include a first fold and a second fold and an overlapping folded portion 118 extending circumferentially between the first and second folds. The overlapping folded portion 118 can be formed to include overlap in a radial direction of at least two thicknesses of the inner layer 108. The inner layer 108 can be extruded, including the folded portion 118. Alternatively, the folded portion 118 can be formed after the inner layer 108 is extruded (e.g., formed on a cylindrical tubular structure).

A discontinuous outer layer 110 can be further provided (at least partially) around the inner layer 108. The outer layer 110 is formed to include an overlapping portion 120 and an underlaying portion 122 such that at least a portion of the folded portion 118 of the inner layer 108 is positioned between the overlapping portion 120 and the underlaying portion 122. In some aspects, the inner layer 108 and the outer layer 110 are coextruded. In alternative aspects, the inner layer 108 and the outer layer 110 are separately formed and joined together.

An adhesive layer 128 can be provided between the inner layer 108 and the outer layer 110 for bonding (at least partially) the inner layer 108 to the outer layer 110. The adhesive layer 128 can be applied to and bond the inner and outer layers 108, 110 axially along a length of the sheath, e.g., along a portion of the entire length of the sheath or along the entire length of the sheath. In an example, the adhesive layer 128 is coextruded with the outer layer 110. In a further example, the adhesive tie layer 128 is coextruded with the inner layer 108. In an alternate example method, the adhesive layer 142 can be applied to the outer surface of the inner liner 108 and/or an inner surface of the outer layer 110.

After the adhesive layer 128 is applied to the desired locations along the inner and/or outer layer 110, the outer layer 110 is applied over the inner layer 108. The outer layer 110 can then be bonded to the inner layer 108 by heat curing the adhesive layer 128. The adhesive layer 128 can comprise a material curable at a temperature above room temperature, in which case a heat treatment may be applied to the assembled inner layer 108/outer layer 110. The adhesive layer 128 may also be composed of a material that cures at room temperature. Accordingly, after the application of the adhesive layer 128 and assembly of the outer layer 110 over the inner layer 108 (at a temperature below room temperature), the temperature of the combined layers may be increased to room temperature.

As illustrated in FIG. 56, the lubricant 142 can be selectively applied to the desired location along a length of outer layer 110 and/or outer jacket (the elongated tube, as described) 140. It is understood that the outer jacket (elongated tube) 140 can be formed by any of the methods disclosed above.

As described above, the lubricant 142 can be provided on an outer surface of the outer layer 110 proximate to a longitudinally extending edge 126 of the overlapping portion 120, on any portion of the folded portion 118 extending/protruding beyond edge 126, and/or on any portion of the outer surface of the outer layer 110 adjacent the protruding portion of the folded portion 118. The lubricant 142 can be applied as a band extending around a portion of the circumference of the outer layer, the band of lubricant 142 also extending longitudinally along a length of the outer layer 110. After the lubricant 142 is selectively applied to the outer surface of the outer layer 110, the outer jacket 140 can be applied over the outer layer 110. As outlined above, the lubricant 142 can comprise a heat-curable material, in which case a heat treatment can be applied to the outer layer 110/outer jacket (elongated tube) 140. The lubricant 142 may also be composed of a material that cures at room temperature. Accordingly, after application of the lubricant 142 (at a temperature below room temperature), the temperature of the outer layer 110 (separately or in combination with the outer jacket (elongated tube) 140) can be increased to room temperature.

The outer jacket 140, formed by any of the methods disclosed above, can then be applied over/around the outer layer 110 and bonded to the outer layer 110 at at least one of the proximal and distal ends of the outer layer 110. The outer jacket 140 can also be bonded to the outer layer 110 along a length of the outer layer 110. The outer jacket 140 can be bonded to the outer layer 110 via a heat treatment process, e.g., a reflow process where the outer layer 110 and the outer jacket are headed to a temperature high enough such that the outer layer 110 and the outer jacket 140 are at least partially melted and are then fused together are the heat is removed, and the assembly cools. The entire sheath 100 assembly may be reflowed to reduce the overall outer diameter and regain/ensure a circular shape in cross-section.

As described above, select portions of the outer surface of the inner liner 108 can include a surface treatment such as surface etching. In an example method, surface treatment of the inner layer 108 would occur before application of the outer layer 110. It is contemplated that it may be desirable to exclude etching from those surfaces of the inner layer 108 that come into contact with the outer surface of the outer layer 110. For example, etching may not be included between the inner surface of the folded portion 118 of the inner layer 108 and the underlaying portion 122 of the outer layer 110. By excluding etching on the portions where the inner layer 108 and the outer surface of the outer layer 110 are in direct contact helps to facilitate release of the inner surface of the folded portion 118 and the outer layer 110 during expansion of the sheath 100.

In some instances, it may be necessary to release (“break”) any undesirable bonding that occurs between the outer layer 110 and the inner layer 108. This bonding can occur due to the etching on the outer surface of the inner liner 108 that allows it to stick directly to the outer layer 110 (with/without the adhesive layer 128). Undesirable bonding can also occur if the outer layer 110 can flows on top of the folded inner layer 108 and “grabs” it during the reflow process.

However, when the inner layer 108 is unetched along those locations excluding the adhesive layer 128, e.g., those portions adjacent the underlaying portion of the outer layer 110, undesirable bonding between the outer layer 110 and the inner layer 108 at this location is limited. Therefore, it may not be necessary to precondition the sheath to release undesirable bonding between the inner layer 108 and the outer layer 110 because the bonding has not occurred (or is less likely to occur).

Nonetheless, undesirable bonding between the inner layer 108 and the underlaying portion 122 of the outer layer 110 can be released while maintaining desirable bonding at the proximal and distal ends of the sheath 100. For example, a mandrel can be passed at least partially through the lumen 116 of the inner layer 108, expanding the inner layer 108 and the outer layer 110, and breaking/releasing any undesirable bonding between the inner layer 108 and the underlaying portion 122 of the outer layer 110.

Various methods can be used to produce the sheaths discussed above and below throughout the present disclosure. For example, a method of making the sheath shown in FIGS. 2A-2D can comprise providing a mandrel and applying an inner layer on the mandrel, such as by spray coating or dip coating the mandrel. An intermediate layer, such as a mesh structure, can then be mounted on the inner layer. An outer layer can be applied over the intermediate layer, such as by a second spray coating or dip coating step. Methods can comprise etching or surface treating at least a portion of the inner layer. Also, methods can comprise providing one or more notches and/or cuts in the inner layer and/or the outer layer. Cuts and/or notches can be provided by, for example, laser cutting or etching one or more layers.

In some aspects of methods of making a sheath, such as the sheaths illustrated in FIGS. 2A-2D, layers can be pre-formed and mounted on a mandrel and then fused or thermally bonded together. For example, in one method, an inner layer is applied to a mandrel. An intermediate layer can be applied to the outer surface of the inner liner. An outer layer can be applied to the outer surface of the intermediate layer. Heat shrink tubing can be applied and the assembly heated, such that the inner layer, the intermediate layer, and/or the outer layer are thermally bonded and compressed together under the heat shrink tubing.

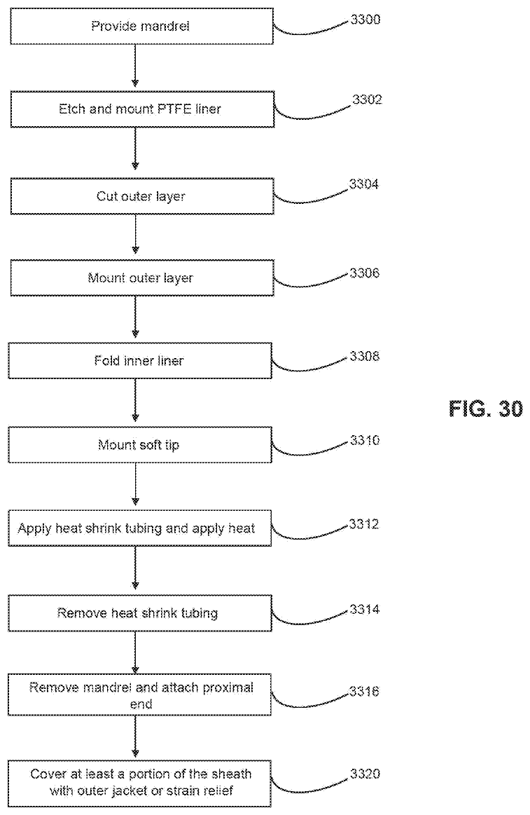

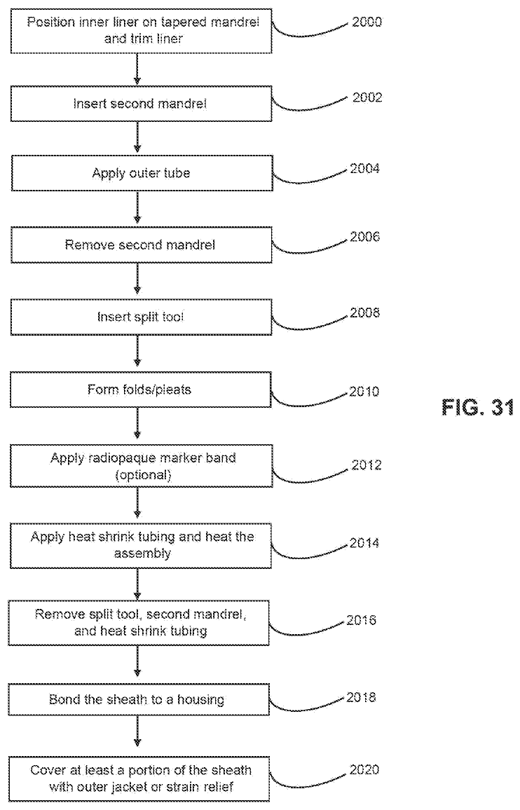

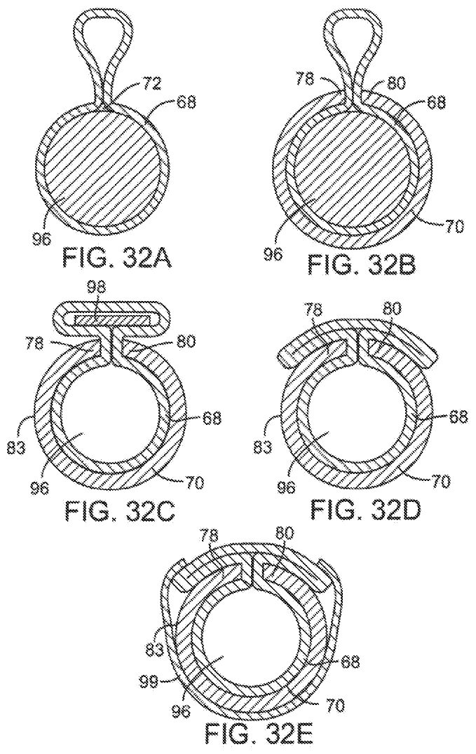

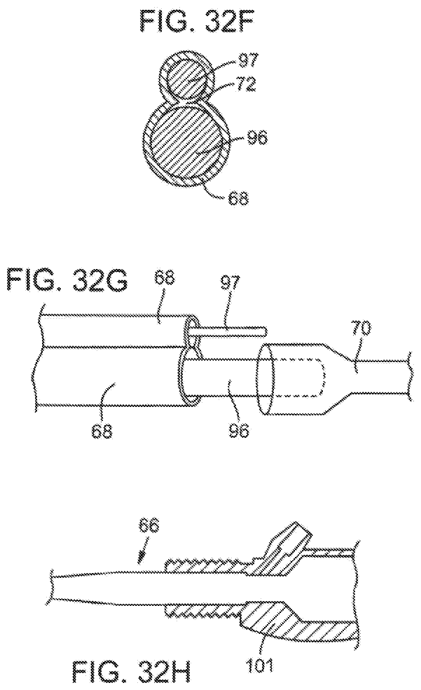

FIG. 30 illustrates a block diagram of one method of producing a sheath for use with a delivery apparatus in minimally invasive surgery. One or more mandrels can be provided (step 3300). The mandrel can be provided with an exterior coating, such as a Teflon® coating, and the mandrel's diameter can be predetermined based on the desired size of the resulting sheath. A liner that will become the inner polymeric layer of the sheath, such as a PTFE or high-density polyethylene liner, can be mounted on the mandrel (step 3302). The liner can be etched and/or surface treated prior to being mounted on the mandrel, according to conventional etching and surface treatment methods. FIG. 32A illustrates a section view of a sheath at steps 3300 and 3302 of FIG. 30. A coated mandrel 96 is inserted within the lumen 72 of the inner polymeric liner 68. The circumference of the inner polymeric liner 68 is larger than the circumference of the mandrel 96, such that an excess portion of the inner polymeric liner 68 can be gathered above the mandrel 96.

A layer of material that will become the outer polymeric tubular layer, such as a layer comprising polyurethane or polyolefin, can be cut or notched through all, substantially all, or a part of the thickness of the layer (step 3304). Such a cut or notch can extend longitudinally along the length of the layer and can extend along substantially the entire length of the outer polymeric tubular layer. In alternative aspects, the cut or notch can be provided along only a portion of the outer polymeric tubular layer. For example, the outer polymeric tubular layer can be cut starting at the distal end of the outer polymeric tubular layer, with the cut ending before the proximal end of the outer polymeric tubular layer. In one aspect, the cut can end at a transition, where the outer diameter of the outer polymeric tubular layer increases or decreases. In one specific aspect, the cut or notch can extend longitudinally along about 75% of the length of the sheath.

The cut or notched outer polymeric tubular layer can be applied, positioned, adhered, mounted, thermally fused or bonded, dip coated, and/or otherwise coupled to the etched inner liner (step 3306). FIG. 32B shows a section view of the sheath at step 3306 of FIG. 30, with outer polymeric tubular layer 70 applied to the inner polymeric liner 68 such that a portion of the inner polymeric liner 68 extends between the cut formed between first and second portions 78, 80 of the outer polymeric tubular layer 70.

In alternative aspects, the outer polymeric tubular layer can be notched or cut after being mounted on the inner liner/mandrel assembly. The outer polymeric tubular layer can optionally be provided with a hydrophilic coating and/or provided with additional layers, such as being dip coated with polyurethane. Some portion of the inner liner can protrude through the cut in the outer polymeric tubular layer after such an outer polymeric tubular layer is mounted onto the inner liner/mandrel arrangement. Using, for example, a split tool, the protruding portion of the inner liner can be folded down onto the outer surface of the outer polymeric tubular layer (step 3308). In some aspects, the protruding portion of the inner liner is folded down along the entire length of the resulting sheath, while in other aspects, the protruding portion of the inner liner is only present along a portion of the length of the sheath or is only folded down along a portion of the length of the resulting sheath. FIG. 32C shows a section view of the sheath at step 3308 of FIG. 30. A split tool 98 is used to fold the excess portion of inner polymeric liner 68 over a portion of the outer surface 83 of the outer polymeric tubular layer 70. FIG. 32D shows a section view of the sheath after completion of step 3308 of FIG. 30. Split tool 98 has been removed, and folding of the excess portion of the inner polymeric liner 68 has been completed. FIG. 32E shows a section view of an outer covering, such as outer polymeric covering 99, that can be applied such that it overlaps a portion of the folded portion of inner polymeric liner 68. The outer polymeric covering 99 contacts at least a portion of the outer surface 83 of the outer polymeric tubular layer 70.

A soft, atraumatic tip can be provided at the distal end of the resulting sheath (step 3310). Additional outer layers can also be applied if desired. Then, a layer of heat shrink tubing, such as fluorinated ethylene propylene (FEP) heat shrink tubing, can be positioned over the entire assembly (step 3312). An appropriate amount of heat is applied, thus shrinking the heat shrink tubing and compressing the layers of the sheath together, such that components of the sheath can be thermally bonded or fused together where desired. Once the components of the sheath have been bonded together, the heat shrink tubing can be removed (step 3314). The proximal end of the sheath can be adhered or otherwise attached to a housing of a catheter assembly, and the sheath can be removed from the mandrel (step 3316). The disclosed herein elongated tube is then positioned (step 3320) on the sheath to form an outer jacket or strain relief jacket. It is understood that the disclosed tube can be positioned at at least a portion of the sheath, for example, and without limitation, the proximal end of the sheath, or it can be positioned along the entire length of the sheath.

FIG. 31 illustrates a block diagram of an alternative aspect of a method of making a sheath. An inner liner, such as an etched PTFE tubing, can be applied to a tapered mandrel, such as a 16 Fr tapered mandrel, and trimmed to an appropriate length (step 2000). A second mandrel, such as a 0.070 inches diameter mandrel, can be inserted in the lumen of the inner liner such that the mandrels are arranged side by side in the inner liner (step 2002). FIG. 32F shows a section view of a sheath at steps 2000 and 2002 of FIG. 31. An inner liner or inner polymeric liner 68 is applied on a first, tapered mandrel 96. A second mandrel 97 is inserted into the lumen 72 of the inner polymeric liner 68 created by the excess portion of the inner polymeric liner 68, as described.

A notched or cut outer polymeric tubular layer, such as high-density polyethylene tubing that has been notched or cut longitudinally, can be slid onto the tapered mandrel and a portion of the inner liner, starting at the distal end of the tapered mandrel (step 2004). The second mandrel can then be removed (step 2006). FIG. 32G illustrates a perspective view of the sheath at steps 2004 and 2006 of FIG. 31. An outer polymeric tubular layer 70 having a longitudinal cut is applied over the tapered mandrel 96 and inner polymeric liner 68. The outer tubular layer conforms to the portion of the inner polymeric layer around the tapered mandrel 96, and the portion of the inner polymeric liner 68 around the second mandrel 97 extends through the longitudinal cut in the outer polymeric tubular layer 70.

A split tool can be inserted into the portion of the lumen of the inner liner that was previously occupied by the second mandrel (step 2008). The split tool can then be used to form folds and/or pleats in the excess portion of the inner liner, which now extends through the longitudinal cut in the outer polymeric tubular layer (step 2010). A radiopaque marker band can optionally be applied at the distal end of the sheath (step 2012). Heat shrink tubing, such as FEP heat shrink tubing, can be applied over the entire sheath, and heat can be applied to compress the components of the sheath and bond or fuse them together (step b). The split tool, heat shrink tubing, and second mandrel can then be removed (step 2016). The sheath can then be utilized with a delivery apparatus, such as by bonding the proximal end of the sheath to a polycarbonate housing of a delivery apparatus or catheter assembly (step 2018).

FIG. 32H illustrates an elevation view of the sheath at step 2018 of FIG. 31. The sheath 66, made according to described methods and processes, can be attached or bonded to a housing 101, such as by bonding the proximal end of the sheath 66 to the polycarbonate housing.

In another example, disclosed expandable sheaths can be made using a reflowed mandrel process. A mandrel can be provided, with the size of the mandrel defining the inner diameter of the sheath lumen in its resting configuration. A tube of material, such as a PTFE tube that will become the sheath's inner liner, can be provided with an inner diameter greater than that of the mandrel (e.g., a 9 mm PTFE tube can be mounted on a 6 mm mandrel). The PTFE tube can be mounted on the mandrel and prepared into the final folded configuration by folding the excess material of the PTFE tube over to one or both sides. An HDPE tube that will serve as the outer layer can then be placed over the PTFE liner. The two-layer assembly can then be thermally fused together. For example, a reflow process can be performed where the assembly is heated to a temperature high enough such that the inner and/or outer layers are at least partially melted and are then fused together as the heat is removed and the assembly cools.

An elongated tube, as described herein, is then placed over at least part of the fused layers (e.g., over a proximal section of the sheath) and held in place using a thermal process (step 2020) to form an outer jacket or a strain relief jacket. In some aspects, the same thermal process can bond the layers of the sheath and the elongated tube. In other aspects, a first thermal process can be used to fuse the layers of the sheath, and a second thermal process can be used to secure the elongated tube to other layers of the sheath. In still further aspects, the elongated tube can cover the whole length of the sheath or at least a portion of the sheath. In some aspects, a distal soft tip can then be attached to the shaft of the expandable sheath.

In some aspects, the outer layer can be co-extruded with an adhesive layer, such as a layer formed from Tecoflex™, such that the Tecoflex™ is positioned on an inner surface of the outer layer—in this manner, the Tecoflex™ will be positioned between the inner and outer layers in the completed sheath. In these aspects, an HDPE tube can be provided with a coating of Tecoflex™ on the inner surface. The HDPE tube can be slit along the length of the tube to open and flatten it and then cut using a template in some aspects. For example, for specific applications, portions of the outer layer can be cut and removed using a template. The cut HDPE can then be placed on the inner layer on the mandrel. In some aspects, only a portion of the outer layer will have the adhesive Tecoflex™. In these aspects, the sections without Tecoflex™ will only be partially fused to the inner layer. In some aspects, the entire inner surface of the outer layer will have the Tecoflex™, and the inner surface of the outer layer can be positioned so that it contacts the inner layer on the mandrel. To position the inner and outer layers, as shown in the sheath of FIG. 39, the folded portion of the inner layer can be lifted up, and an edge of the outer layer can be tucked beneath the fold.

Some additional sheaths, as disclosed herein, can also be prepared according to the methods disclosed below. For example, also disclosed are aspects where the sheath is formed by forming a variable diameter inner liner by rolling a sheet having a first edge and a second edge and wherein the sheet is defined by an inner surface and an outer surface in a spiral configuration such that at least a portion of the inner surface of the sheet overlays at least a portion of the outer surface of the sheet thereby forming an overlying portion and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis. The elongated tube formed as disclosed above can be used to form an outer layer of the sheath. In such aspects, the outer layer will comprise and the disclosed herein elongated tube and a braid.

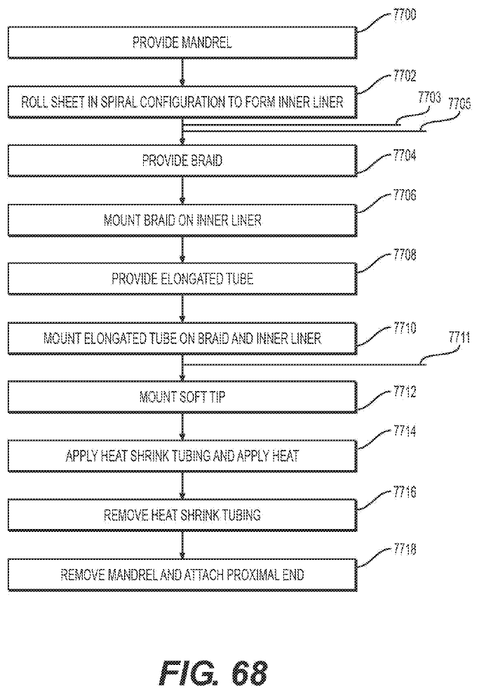

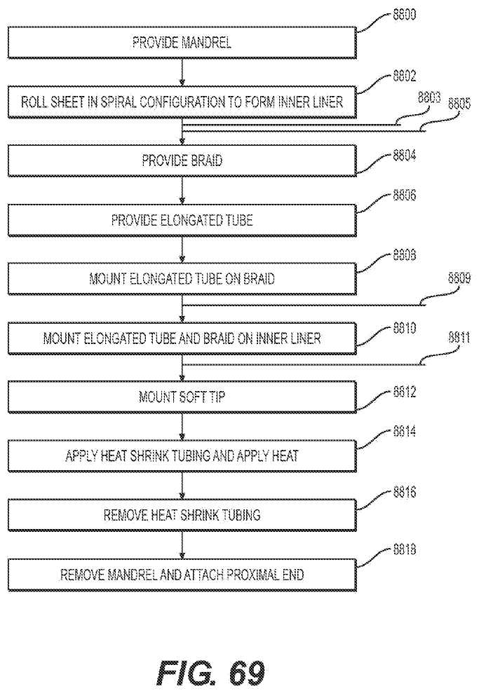

FIGS. 68 and 69 exemplify block diagrams of exemplary methods of producing the sheath in various aspects. The various methods steps are also depicted in FIGS. 70A-70J. In certain aspects, and as shown in FIG. 70A, the inner liner can be formed from an extruded tube 1903, having an inner surface and outer surface and having any thickness that is described above. This extruded tube can be cut 1905 along the length to form a sheet. In certain aspects, the inner surface and/or outer surface of the tube can be surface-treated, such as, for example, by plasma etching, chemical etching, or other suitable methods of surface treatment. In some exemplary aspects, where the outer surface of the inner liner is treated, the treatment can provide for better bonding with the outer layer when formed. In yet other aspects, the inner surface of the inner liner can be ribbed. In such exemplary aspects, the ribbed surface facilitates a reduction of contact points with the prosthetic device and can reduce friction. In still further aspects, the initial extruded tube 1903 can be produced by co-extrusion with multiple layers of the same or different polymers as described herein. It is understood that one of ordinary skill in the art can choose the composition of the inner liner depending on the desired application. In certain aspects, the decision to use a specific material for the inner liner can be dependent on the desired stiffness, wall-thickness, and lubricious optimization.

In still further aspects, one or more mandrels can be provided (step 7700 or 8800 in FIGS. 68 and 69, respectively). The mandrel can be provided with an exterior coating, such as a Teflon® coating, and the mandrel's diameter can be predetermined based on the desired rest diameter dᵣ of the resulting sheath. As shown in FIG. 70B, the sheet formed by cutting 1905 the extruded tube 1903 can be rolled in a spiral configuration (steps 7702 and 8802 in FIGS. 68 and 69 respectively) around the mandrel 1901 to form the inner liner 1902a such that at least a portion of the inner surface of the sheet overlays at least a portion of the outer surface of the sheet thereby forming an overlaying portion 1902c and wherein the first edge (not shown) of the sheet is slidable along at least a portion the inner surface of the sheet and the second edge 1902b is slidable along at least a portion of the outer surface of the sheet.

In still further exemplary aspects, in steps 7705 and 8805 (FIGS. 68 and 69 respectively), an amount of a first lubricant 1910 (FIGS. 70C-70E) can be optionally applied on the outer surface of the inner liner. The presence of this lubricant material can reduce the friction between the inner liner and the outer layer of the final sheath. In yet other aspects, in steps 7703 and 8803, an amount of a second lubricant 1908 can be applied between the overlaying and sliding portions of the inner liner to further improve slidability and decrease friction. (FIG. 70D depicts the inner liner with the two optional lubricants present with the mandrel hidden from the view). In still further aspects, it is understood that the inner liner formed with the use of a mandrel can have any rest diameter, as described above. In certain aspects, the rest diameter dᵣ is substantially uniform along the longitudinal axis of the lumen. While in the other aspects, the rest diameter dᵣ varies along the longitudinal axis of the lumen and wherein the rest diameter dᵣ at the proximal end that is larger than the rest diameter dᵣ at the distal end.

In still further aspects, the methods can further comprise a step of providing a braid (steps 7704 and 8804). It is understood that any of the described above braids can be used in this step. In still further aspects, and as shown in step 7706 of FIG. 68, the braid is mounted on the inner liner. In some exemplary aspects, and as shown in FIG. 70F, the braid 1904 can be mounted on the first lubricant 1910 that can be present on the outer surface of the inner liner. It is understood that in some aspects, the second lubricant can be present only at a portion of the outer surface of the inner liner. In yet other aspects, the disclosed sheath can have segments where the first lubricant is present, and the braid is mounted over it, while it can have other segments where the second lubricant is not present, and the braid is mounted directly on the outer surface of the inner liner. It is understood that the location of these specific segments can be determined by one of ordinary skill in the art depending on the desired application. It is understood that the mounting of the braid can be done by any known in the art methods. In some unlimiting aspects, the braid can be provided as a cylindrical tube, and it can be slid on top of the inner liner or the first lubricant if it is present.

In yet further aspects and as shown in step 7708, the method can further comprise a step of providing the disclosed herein elongated tube. In certain and unlimiting aspects, the elongated tube is extruded from the disclosed herein compositions and is provided as a cylindrical tube 1906 (FIG. 70G). In a still further aspect, the disclosed elongated tube can be mounted on the inner liner and the braid (step 7710). FIG. 70G, for example, depicts an aspect where the disclosed herein elongated tube 1906 is used to slide on the inner liner having a first lubricant 1910 overlaying the inner liner's outer surface and the braid 1904.

In yet further aspects, the disclosed method can comprise a step of embedding (step 7711, FIG. 68) the braid into the elongated tube. It is understood that the sheath can comprise various segments. In some aspects, some of the segments can comprise the braid embedded within the elongated tube, while in other segments, the braid and the elongated tube are separate. It is further understood that in some aspects, the sheath can have a braid embedded within the elongated tube over the whole length of the sheath, while in other aspects, the braid is not embedded within the elongated tube over the whole length of the sheath. It is further understood that any methods known in the art can be used to embed the braid within the elongated tube. In some aspects, application of the heat can be utilized. In certain aspects, the use of heat shrink tubing can be utilized to embed the braid within the disclosed herein elongated tube. It is understood that after the step of embedment is complete, the heat shrink tubing is removed.

In still further aspects, a soft, atraumatic tip can be provided at the distal end of the resulting sheath (step 7712). In yet further aspects, the outer layer comprising the braid and the layer of the elastomeric polymer is at least partially bonded to the inner liner. It is understood that this bonding can also be achieved by any known in the art methods. In certain aspects, and as shown in step 7714, a heat shrink is applied to the portion that is being bound and heated to form a bonding between the inner liner and the outer layer. In yet other aspects, the bonding between the inner liner and the outer layer can be achieved by placing the assembly in an oven or otherwise heating it. In still further aspects, the bonding is performed by heating at a temperature from about 350° F. to about 550° F. for a time period effective to form a bond between at least a portion of the outer layer and at least a portion of the inner liner. In yet further aspects, the heating can be done at a temperature of about 375° F., about 400° F., about 425° F., about 450° F., about 475° F., about 500° F., or about 525° F. In yet other aspects, the time period effective to form a bond can comprise from about 1 second to about 60 seconds, including exemplary values of about 5 seconds, about 10 seconds, about 15 seconds, about 20 seconds, about 25 seconds, about 30 seconds, about 35 seconds, about 40 seconds, about 45 seconds, about 50 seconds, and about 55 seconds. However, it is further understood that this time period is not limiting, and it can have any value needed to provide for an effective bond, for example, it can have any value from about 1 second to about 5 hours. It is further understood that if the heat shrink tubing is used to obtain the desired bonding, the heat shrink tubing is removed (step 7716, FIG. 68).

In still further aspects and as shown in FIG. 701, the bonding step can also comprise applying a first strip 1920 of a polymer along at least a portion of the longitudinal axis of the lumen to at least a portion of the outer surface of the sheet that does not comprise the overlaying portion 1902c prior to or during the step of bonding the at least a portion of the inner surface of the elongated tube to at least a portion of the outer surface of the sheet of the inner liner. It is understood that in some aspects, the first strip of the polymer can be made of the same material as the elongated tube itself. While in other aspects, the first strip of the polymer can be made of any material that allows an efficient bonding between the inner liner and the elongated tube. It is understood that in some exemplary aspects and as shown in the FIG. 70I, this first strip can be applied prior to mounting the braid. In still further aspects, the location where the first strip is applied does not comprise the first lubricant. However, it is understood that in such aspects, the first lubricant can be present in other locations.

In still further exemplary aspects, the methods can comprise applying a second strip 1922a of a polymer to at least a portion of the outer surface of the sheet at the proximal end of the sheath prior to or during the step of bonding the at least a portion of the inner surface of the disclosed herein elongated tube to at least a portion of the outer surface of the sheet of the inner liner. It is understood that in some aspects, the second strip of the polymer can be made of the same material as the elongated tube itself. While in other aspects, the second strip of the polymer can be made of any material that allows an efficient bonding between the inner liner and the elongated tube. It is further understood that the first and the second strips can be made from the same or different polymers.

It is understood that in some exemplary aspects and as shown in the FIG. 70J, this second strip can be applied prior to mounting the braid. In still further aspects, the location where the second strip is applied does not comprise the first lubricant. However, it is understood that in such aspects, the first lubricant can be present in other locations. In yet other aspects, the method can comprise a third strip of polymer 1922b that can be applied to at least a portion of the outer surface of the sheet at the distal end of the sheath prior to or during the step of bonding the at least a portion of the inner surface of the disclosed herein elongated tube to at least a portion of the outer surface of the sheet of the inner liner. It is understood that in some aspects, the third strip of the polymer can be made of the same material as the elongated tube itself. While in other aspects, the third strip of the polymer can be made of any material that allows an efficient bonding between the inner liner and the elongated tube. It is further understood that the first, the second, and/or third strips can be made from the same or different polymers.

It is understood that in some exemplary aspects and as shown in the FIG. 70J, this third strip can be applied prior to the mounting the braid. In still further aspects, the location where the third strip is applied does not comprise the first lubricant. However, it is understood that in such aspects, the first lubricant can be present in other locations. In still further aspects, both the second and third strips are present. While in other aspects, only one of the second or the third strips is present. It is further understood that the first, second, and third strips can be made from the same or different polymers.

Some alternative aspects are shown in FIG. 69 and FIG. 70H. In such alternative aspects, the outer layer is pre-formed by combining the elongated tube and braid together and then mounted on the inner liner positioned on the mandrel. In such aspects, the disclosed elongated tube is first mounted on the braid (step 8808) prior to the mounting it on the inner liner. In yet other aspects, the method can also comprise a step of partially embedding the braid within the elongated tube before mounting both of them on the inner liner (step 8809). However, the step of partially embedding the braid with the elongated tube can be done after the braid and the elongated tube are mounted on the inner liner (step 8811). Steps 8812-8818 can be performed analogously to the steps 7712-7718.

In still further aspects, and as shown in FIG. 701, the bonding step can also comprise a first strip 1920 being applied along at least a portion of the longitudinal axis of the lumen to at least a portion of the outer surface of the sheet that does not comprise the overlaying portion 1902c prior to or during the step of bonding the at least a portion of the inner surface of the elongated tube to at least a portion of the outer surface of the sheet of the inner liner. It is understood that in some exemplary aspects and as shown in the FIG. 701, this first strip can be applied prior to the mounting pre-formed outer layer comprising the braid and the elongated tube. In still further aspects, the location where the first strip is applied does not comprise the first lubricant. However, it is understood that in such aspects, the first lubricant can be present in other locations.

It is also understood that these alternative aspects can also include a step where a second strip 1922a can be applied to at least a portion of the outer surface of the sheet at the proximal end of the sheath prior to or during the step of bonding the at least a portion of the inner surface of the elongated tube to at least a portion of the outer surface of the sheet of the inner liner. It is understood that in some exemplary aspects and as shown in the FIG. 70J, this second strip can be applied prior to the mounting pre-formed outer layer comprising the braid and the elongated tube. In still further aspects, the location where the second strip is applied does not comprise the first lubricant. However, it is understood that in such aspects, the first lubricant can be present in other locations. In yet other aspects, these methods can also comprise a third strip 1922b that can be applied to at least a portion of the outer surface of the sheet at the distal end of the sheath prior to or during the step of bonding the at least a portion of the inner surface of the disclosed elongated tube to at least a portion of the outer surface of the sheet of the inner liner. It is understood that in some exemplary aspects and as shown in the FIG. 70J, this third strip can be applied prior to the mounting pre-formed outer layer comprising the braid and the elongated tube. In still further aspects, the location where the third strip is applied does not comprise the first lubricant. However, it is understood that in such aspects, the first lubricant can be present in other locations. In still further aspects, both the second and the third elastomeric polymers are present.

Again, it is understood that the method that does not include the application of the braid is also disclosed. In such methods, the elongated braid as disclosed herein forms the outer layer of the sheath, and the braid is not present.

Also disclosed herein are aspects where the disclosed herein methods can comprise a step of disposing a hydrophilic coating layer on the outer surface of the elongated tube of any of the exemplary sheaths. Any disclosed herein hydrophilic coating can be used.

Any of the disclosed herein sheaths can also be attached to the housing 101, as shown in FIG. 32H or a soft tip 102 as, for example, shown in FIG.36.

Sheaths of the present disclosure can be used with various methods of introducing a prosthetic device into a patient's vasculature. One such method comprises positioning an expandable sheath in a patient's vessel, passing a device through the introducer sheath, which causes a portion of the sheath surrounding the device to expand and accommodate the profile of the device, and automatically retracting the expanded portion of the sheath to its original size after the device has passed through the expanded portion. In some methods, the expandable sheath can be sutured to the patient's skin at the insertion site so that once the sheath is inserted the proper distance within the patient's vasculature, it does not move once the implantable device starts to travel through the sheath.

Disclosed aspects of an expandable sheath can be used with other delivery and minimally invasive surgical components, such as an introducer and loader. In one aspect, the expandable sheath can be flushed to purge any air within the sheath, using, for example, flush port 103 (FIG. 35). An introducer can be inserted into the expandable sheath, and the introducer/sheath combination can be fully inserted into vasculature over a guiding device, such as a 0.35″ guidewire. Preferably, the seam formed by the intersection of the folded portion of the inner layer and the overlapping portion of the outer layer can be positioned such it is oriented downward (posterior). Once the sheath and introducer are fully inserted into a patient's vasculature, in some aspects, the expandable sheath can be sutured in place at the insertion site. In this manner, the expandable sheath can be substantially prevented from moving once positioned within the patient.

The introducer can then be removed, and a medical device, such as a transcatheter heart valve, can be inserted into the sheath, in some instances, using a loader. Such methods can additionally comprise placing the tissue heart valve in a crimped state on the distal end portion of an elongated delivery apparatus and inserting the elongated delivery device with the crimped valve into and through the expandable sheath. Next, the delivery apparatus can be advanced through the patient's vasculature to the treatment site, where the valve can be implanted.

Typically, the medical device has a greater outer diameter than the diameter of the sheath in its original configuration. The medical device can be advanced through the expandable sheath towards the implantation site, and the expandable sheath can locally expand to accommodate the medical device as the device passes through. The radial force exerted by the medical device can be sufficient to locally expand the sheath to an expanded diameter (e.g., the expanded configuration) just in the area where the medical device is currently located. Once the medical device passes a particular location of the sheath, the sheath can at least partially contract to the smaller diameter of its original configuration. The expandable sheath can thus be expanded without the use of inflatable balloons or other dilators. Once the medical device is implanted, the sheath and any sutures holding in place can be removed. In some aspects, it is preferable to remove the sheath without rotating it.

### EXEMPLARY ASPECTS

### EXAMPLE 1

A sheath for delivering a medical device, wherein the sheath has a proximal and a distal end and comprises an elongated tube forming an outer layer of the sheath that is positioned at at least the proximal end of the sheath and extending along at least a portion of a length of the sheath, having an inner surface and an outer surface, and wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; wherein the elongated tube is configured to reversibly expand from an initial diameter do in an unexpended position to an expanded diameter dₑ in an expanded position upon passage of a medical device; and wherein the sheath exhibits at least a 10% reduction in an insertion force when compared with a substantially identical reference sheath that does not comprise the first polymer layer.

### EXAMPLE 2

The sheath of any examples herein, particularly example 1, wherein the first polymer has a substantially same durometer along a total length of the elongated tube.

### EXAMPLE 3

The sheath of any examples herein, particularly example 1, wherein a durometer of the first polymer at a proximal end of the elongated tube is different from a durometer of the first polymer at a distal end of the elongated tube.

### EXAMPLE 4

The sheath of any one of examples herein, particularly examples 1-3, wherein the first polymer has a Shore D from about 20 D to about 35 D.

### EXAMPLE 5

The sheath of any examples herein, particularly examples 1-4, wherein the first polymer comprises PEBAX®.

### EXAMPLE 6

The sheath of any examples herein, particularly examples 1-4, wherein the first polymer comprises polyurethane.

### EXAMPLE 7

The sheath of any examples herein, particularly examples 1-6, wherein the inorganic filler comprises bismuth oxychloride, barium sulfate, bismuth subcarbonate, calcium carbonate, aluminum trihydrate, barite, kaolin clay, limestone, or any combination thereof.

### EXAMPLE 8

The sheath of any examples herein, particularly examples 1-7, wherein the inorganic filler is present in an amount of at least about 10% based on a total weight of the first compound composition.

### EXAMPLE 9

The sheath of any examples herein, particularly examples 1-8, wherein the inorganic filler is present in an amount of less than about 50% based on a total weight of the first compound composition.

### EXAMPLE 10

The sheath of any examples herein, particularly examples 1-9, wherein the solid lubricant comprises a PTFE filler.

### EXAMPLE 11

The sheath of any examples herein, particularly example 10, wherein the PTFE filler is a powder.

### EXAMPLE 12

The sheath of any examples herein, particularly examples 1-11, wherein the first compound composition further comprises at least one tackiness reducing compound.

### EXAMPLE 13

The sheath of any examples herein, particularly example 12, wherein the at least one tackiness reducing compound is present in an amount from about 1% to about 20% based on a total weight of the first compound composition.

### EXAMPLE 14

The sheath of any examples herein, particularly example 12 or 13, wherein the at least one tackiness reducing compound comprises ProPell™.

### EXAMPLE 15

The sheath of any examples herein, particularly examples 1-14, wherein the elongated tube comprises two or more polymer layers.

### EXAMPLE 16

The sheath of any examples herein, particularly example 15, wherein the elongated tube comprises at least a second polymer layer comprising a second compound composition comprising from greater than 0 wt % to 100 wt % of a second polymer comprising polyether block amide, a polyurethane, or a composition thereof.

### EXAMPLE 17

The sheath of any examples herein, particularly example 16, wherein the second compound composition further comprises up to 20% of tackiness reducing additive based on a total weight of the second compound composition.

### EXAMPLE 18

The sheath of any examples herein, particularly examples 15-17, wherein the second polymer layer comprises PEBAX®.

### EXAMPLE 19

The sheath of any examples herein, particularly examples 15-17, wherein the second polymer layer comprises polyurethane.

### EXAMPLE 20

The sheath of any examples herein, particularly examples 15-19, wherein the second polymer has a Shore A Durometer from about 20 A to about 60 A.

### EXAMPLE 21

The sheath of any examples herein, particularly examples 15-20, wherein the second compound composition is substantially free of an inorganic filler.

### EXAMPLE 22

The sheath of any examples herein, particularly examples 15-21, wherein the second compound composition is substantially free of a lubricant solid.

### EXAMPLE 23

The sheath of any examples herein, particularly examples 15-22, wherein the elongated tube has a predetermined thickness and wherein at least about 50% of the predetermined thickness comprises the first and/or the second compound composition comprising the first and/or the second polymer having a Shore D Durometer from about 20 D to about 35 D.

### EXAMPLE 24

The sheath of any examples herein, particularly example 23, wherein the predetermined thickness is up to 6 mils.

### EXAMPLE 25

The sheath of any examples herein, particularly example 24, wherein the predetermined thickness of the elongated tube varies along a length of the sheath.

### EXAMPLE 26

The sheath of any examples herein, particularly example 25, wherein the predetermined thickness of the elongated tube is greater at the proximal end.

### EXAMPLE 27

The sheath of any examples herein, particularly example 25 or 26, wherein the predetermined thickness of the elongated tube is smaller at the distal end as compared to the predetermined thickness of the elongated tube at the proximal end.

### EXAMPLE 28

The sheath of any examples herein, particularly examples 1-27, wherein the first polymer layer has a thickness of about 1 mil to about 3 mils.

### EXAMPLE 29

The sheath of any examples herein, particularly examples 15-28, wherein the second polymer layer has a thickness of about 2 mils to about 4 mils.

### EXAMPLE 30

The sheath of any examples herein, particularly examples 15-29, wherein the first polymer layer defines the inner surface of the elongated tube.

### EXAMPLE 31

The sheath of any examples herein, particularly examples 15-30, wherein the second polymer layer defines the outer surface of the elongated tube.

### EXAMPLE 32

The sheath of any examples herein, particularly examples 15-31, wherein the first polymer layer defines the outer surface of the elongated tube.

### EXAMPLE 33

The sheath of any examples herein, particularly example 32, wherein the second polymer layer defines the inner surface of the elongated tube.

### EXAMPLE 34

The sheath of any examples herein, particularly examples 15-33, wherein one or more additional polymer layers are disposed between the first polymer layer and the second polymer layer.

### EXAMPLE 35

The sheath of any examples herein, particularly examples 1-34, wherein the sheath exhibits at least about 20% reduction in an insertion force when compared with a substantially identical reference sheath that does not comprise the first polymer layer.

### EXAMPLE 36

The sheath of any examples herein, particularly examples 1-35, wherein the elongated tube is extruded.

### EXAMPLE 37

The sheath of any examples herein, particularly example 36, wherein the first polymer layer and the second polymer layer are co-extruded.

### EXAMPLE 38

The sheath of any examples herein, particularly examples 16-37, wherein the first polymer layer is substantially bonded to the second polymer layer.

### EXAMPLE 39

The sheath of any examples herein, particularly examples 1-38, wherein the elongated tube exhibits a friction force of less than about 10 N in the dry state against a substrate surface comprising one or more of polytetrafluoroethylene, fluorinated ethylene propylene, or high-density polyethylene.

### EXAMPLE 40

The sheath of any examples herein, particularly examples 1-39, wherein the elongated tube exhibits a friction force of less than about 7 N in the dry state against a substrate surface comprising one or more of polytetrafluoroethylene, fluorinated ethylene propylene, or high-density polyethylene.

### EXAMPLE 41

The sheath of any examples herein, particularly examples 1-40, wherein the elongated tube exhibits a hoop direction force at a 10 mm extension of less than about 8 N.

### EXAMPLE 42

The sheath of any examples herein, particularly examples 1-41, the elongated tube exhibits an elongation at break ranging between about 650% and about 800%.

### EXAMPLE 43

The sheath of any examples herein, particularly examples 1-42, wherein the elongated tube is substantially kink resistant.

### EXAMPLE 44

The sheath of any examples herein, particularly examples 1-43, where the elongated tube extends along the length of the sheath.

### EXAMPLE 45

The sheath of any examples herein, particularly examples 1-44, wherein the elongated tube is positioned at the proximal end of the sheath and extends to the distal end of the sheath.

### EXAMPLE 46

The sheath of any examples herein, particularly examples 1-45, further comprising an expandable tubular inner liner extending along the length of the sheath and comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion; and a first outer tubular layer extending at least partially along the length of the sheath and having an inner surface and an outer surface, wherein the inner surface of the first outer tubular layer further extends at least partially around the outer surface of the inner liner such that at least a portion of the inner surface of the first outer tubular layer is positioned adjacent to the outer surface of the at least one folded portion of the inner liner; wherein the elongated tube is positioned such that at least a portion of the inner surface of the elongated tube overlies at least a portion of the outer surface of the first outer tubular layer.

### EXAMPLE 47

The sheath of any examples herein, particularly example 46, wherein the sheath exhibits an insertion force of less than about 55 N when a medical device is pushed through the sheath.

### EXAMPLE 48

The sheath of any examples herein, particularly examples 46-47, wherein the expandable tubular inner liner comprises polytetrafluoroethylene.

### EXAMPLE 49

The sheath of any examples herein, particularly examples 46-48, wherein the first outer tubular layer comprises a high-density polyethylene.

### EXAMPLE 50

The sheath of any examples herein, particularly examples 46-49, wherein the outer surface of the first outer tubular layer is at least partially selectively etched.

### EXAMPLE 51

The sheath of any examples herein, particularly example 50, wherein the outer surface of the inner liner is selectively etched around the circumference, linearly along at least a portion of the length of the sheath, or a combination thereof.

### EXAMPLE 52

The sheath of any examples herein, particularly examples 50-51, wherein at least a portion of the outer surface of the at least one folded portion of the inner liner is not etched along at least a portion of the sheath length.

### EXAMPLE 53

The sheath of any examples herein, particularly examples 50-52, wherein the outer surface of the inner liner comprises one or more nonetched portions along the sheath length.

### EXAMPLE 54

The sheath of any examples herein, particularly example 53, wherein each of the one or more nonetched portions is followed by an etched portion.

### EXAMPLE 55

The sheath of any examples herein, particularly example 53 or 54, wherein the one or more nonetched portions comprise the outer surface of the at least one folded portion.

### EXAMPLE 56

The sheath of any examples herein, particularly examples 50-55, wherein the sheath exhibits an insertion force of less than about 55 N.

### EXAMPLE 57

The sheath of any examples herein, particularly examples 50-56, wherein the sheath exhibits a reduction in an insertion force of at least about 25% when compared to a substantially identical reference sheath that does not comprise the first compound composition and the selectively etched inner liner.

### EXAMPLE 58

The sheath of any examples herein, particularly examples 46-57, wherein the inner liner comprises two or more folded portions.

### EXAMPLE 59

The sheath of any examples herein, particularly examples 46-58, wherein the at least one folded portion comprises a first folded edge and a second folded edge and an overlapping portion extending circumferentially between the first and second folded edges, the overlapping portion comprising an overlap in a radial direction of at least two thicknesses of the inner liner, and wherein the first folded edge is configured to move closer to the second folded edge to shorten the overlapping portion at a local axial location during application of a radial outward force by passage of the medical device and wherein shortening of the overlapping portion corresponds with a local expansion of the lumen.

### EXAMPLE 60

The sheath of any examples herein, particularly examples 46-59, wherein the at least one folded portion comprises a first folded edge and a second folded edge and an overlapping portion extending circumferentially between the first and second folded edges, the overlapping portion comprising an overlap in a radial direction of at least two thicknesses of the inner liner, wherein the first folded edge is configured to move closer to the second folded edge to shorten the overlapping portion at a local axial location during application of a radial outward force by passage of the medical device and wherein shortening of the overlapping portion corresponds with a local expansion of the lumen, and wherein the outer layer includes a first longitudinally extending edge and a second longitudinally extending edge configured to separate as the sheath expands, the first longitudinal extending edge and an overlapping portion of the outer layer extending over the second longitudinally extending edge when the sheath is not expanded.

### EXAMPLE 61

The sheath of any examples herein, particularly examples 46-60, wherein the inner liner is configured to expand to a substantially cylindrical tube.

### EXAMPLE 62

A sheath for delivering a medical device, wherein the sheath has a proximal and a distal end and comprises: an expandable tubular inner liner comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion; a first outer tubular layer having an inner surface and an outer surface, wherein the inner surface of the first outer tubular layer extends at least partially around the outer surface of the inner liner such that at least a portion of the inner surface of the first outer tubular layer is positioned adjacent to the outer surface of the at least one folded portion of the inner liner; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition.

### EXAMPLE 63

A sheath for delivering a medical device, wherein the sheath has a proximal and a distal end and comprises: an expandable tubular inner liner comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion and wherein the outer surface of the inner liner is selectively etched; a first outer tubular layer having an inner surface and an outer surface, wherein the inner surface of the outer layer extends at least partially around the outer surface of the inner liner such that at least a portion of the inner surface of the outer layer is positioned adjacent to at least a portion of the outer surface of the at least one folded portion of the inner liner; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition.

### EXAMPLE 64

The sheath of any examples herein, particularly examples 1-45, further comprising a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and wherein the elongated tube is positioned such that the inner surface of the elongated tube overlies at least a portion of the outer surface of the inner liner.

### EXAMPLE 65

The sheath of any examples herein, particularly example 64, further comprising a braid positioned between the inner liner and the elongated tube.

### EXAMPLE 66

The sheath of any examples herein, particularly example 64 or 65, further comprising an additional outer layer positioned in between the inner liner and the elongated tube.

### EXAMPLE 67

The sheath of any examples herein, particularly example 66, wherein the sheath contracts to the predetermined rest diameter dᵣ after passage of the medical device through the lumen.

### EXAMPLE 68

The sheath of any examples herein, particularly examples 64-67, wherein the sheet comprises a high-density polyethylene, polypropylene, polyamide, fluoropolymer, copolymers thereof, or blends thereof.

### EXAMPLE 69

The sheath of any examples herein, particularly example 68, wherein the sheet has a multilayer structure.

### EXAMPLE 70

The sheath of any examples herein, particularly examples 64-69, wherein the internal surface of the sheet is at least partially ribbed.

### EXAMPLE 71

The sheath of any examples herein, particularly examples 64-70, wherein the sheet is lubricious and has a coefficient of friction less than about 0.5.

### EXAMPLE 72

The sheath of any examples herein, particularly examples 64-71, wherein an amount of a first lubricant is disposed between at least a portion of the inner liner and at least a portion of the inner surface of the elongated tube.

### EXAMPLE 73

The sheath of any examples herein, particularly examples 64-72, wherein an amount of a second lubricant is disposed between at least a portion of the overlying portion of the sheet and at least a portion of the sliding portions of the sheet.

### EXAMPLE 74

The sheath of any examples herein, particularly examples 65-73, wherein the braid comprises at least one filament comprising stainless steel, nitinol, a polymer material, or a composite material.

### EXAMPLE 75

The sheath of any examples herein, particularly examples 65-7, wherein the braid has a per-inch crosses (PIC) count of less than 50.

### EXAMPLE 76

A sheath for delivering a medical device, wherein the sheath has a proximal and a distal end and comprises: a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and an elongated tube forming an outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the inner liner, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition.

### EXAMPLE 77

The sheath of any examples herein, particularly examples 1-45, further comprising an inner tubular layer comprising a longitudinal slit and partially defining an inner lumen, a first outer tubular layer enveloping the inner layer, the outer tubular layer comprising a longitudinally extending, folded flap that overlies a portion of an outer surface of the outer layer when the sheath is in an unexpanded state, and the elongated tube positioned such that the inner surface of the elongated tube overlies at least a portion of the outer surface of the first outer tubular layer.

### EXAMPLE 78

The sheath of any examples herein, particularly example 77, wherein a base of the folded flap is positioned radially outwardly from the longitudinal slit.

### EXAMPLE 79

The sheath of any examples herein, particularly example 77 or 78, wherein the folded flap includes a longitudinally extending overlying portion separated from a longitudinally extending underlaying portion by a longitudinally extending crease.

### EXAMPLE 80

The sheath of any examples herein, particularly example 79, wherein the underlaying portion contacts an outer surface of the outer tubular layer when the sheath is in the unexpanded state.

### EXAMPLE 81

The sheath of any examples herein, particularly examples 78-80, wherein a base of the folded flap extends the length of the outer tubular layer and wherein the overlying portion and the underlaying portion extend between the base and the crease.

### EXAMPLE 82

The sheath of any examples herein, particularly examples 78-81, wherein the overlying portion, the underlaying portion, or both have a wall thickness that is thinner than the remainder of the outer tubular layer.

### EXAMPLE 83

The sheath of any examples herein, particularly examples 78-82, wherein the longitudinally extending flap extends around about 20% to about 40% of an outer circumference of the outer tubular layer when the sheath is in an unexpanded state.

### EXAMPLE 84

The sheath of any examples herein, particularly examples 76-83, wherein the first outer tubular layer comprises a material having a tensile modulus of at least 300 MPa.

### EXAMPLE 85

The sheath of any examples herein, particularly example 84, wherein the first outer tubular layer comprises a material having a tensile modulus from 300 MPa to 2,000 MPa.

### EXAMPLE 86

The sheath of any examples herein, particularly examples 77-85, wherein the first outer tubular layer comprises a material having an ultimate tensile strength of at least 50 MPa.

### EXAMPLE 87

The sheath of any examples herein, particularly examples 77-86, wherein the first outer tubular layer comprises a shape memory material.

### EXAMPLE 88

The sheath of any examples herein, particularly examples 77-87, wherein the first outer tubular layer comprises a polyamide, co-polyamide, polyether block amide (PEBAX®), or a blend.

### EXAMPLE 89

The sheath of any examples herein, particularly examples 76-88, wherein the first outer tubular layer further comprises at least one additional longitudinally extending folded flap that overlies a portion of the outer surface of the outer layer when the sheath is in an unexpanded state.

### EXAMPLE 90

The sheath of any examples herein, particularly examples 76-89, wherein the longitudinal slit extends the full length of the inner tubular layer.

### EXAMPLE 91

The sheath of any examples herein, particularly examples 76-90, wherein the inner tubular layer comprises a first longitudinally extending end and a second longitudinally extending end, the first and second longitudinally extending ends defining the longitudinal slit.

### EXAMPLE 92

The sheath of any examples herein, particularly examples 76-91, wherein the inner tubular layer comprises a material with a static or dynamic coefficient of friction less than 0.3.

### EXAMPLE 93

The sheath of any examples herein, particularly examples 76-92, wherein the inner tubular layer extends around at least 80% of a circumference of the inner lumen when the sheath is in an unexpanded state.

### EXAMPLE 94

The sheath of any examples herein, particularly examples 76-93, wherein the inner tubular layer is formed of a material having a tensile modulus of at least 300 MPa.

### EXAMPLE 95

The sheath of any examples herein, particularly examples 76-94, wherein the inner tubular layer comprises HDPE or a fluoropolymer.

### EXAMPLE 96

The sheath of any examples herein, particularly examples 76-95, further comprising a tie layer positioned between and adhering the inner tubular layer to the first outer tubular layer.

### EXAMPLE 97

The sheath of any examples herein, particularly example 96, wherein the tie layer comprises a polyurethane or functionalized polyolefin.

### EXAMPLE 98

An expandable sheath comprising: an inner tubular layer comprising a longitudinal slit and partially defining an inner lumen, a first outer tubular layer enveloping the inner layer, the outer tubular layer comprising a longitudinally extending, folded flap that overlies a portion of an outer surface of the outer layer when the sheath is in an unexpanded state, and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; wherein an outwardly directed radial force from a prosthetic device moving through the inner lumen widens the longitudinal slit and unfolds the folded flap to allow expansion of the sheath.

### EXAMPLE 99

The sheath of any examples herein, particularly examples 1-45, further comprising a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; and wherein the elongated tube is positioned such that the inner surface of the elongated tube overlies at least a portion of the outer surface of the discontinuous first outer tubular layer ; wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

### EXAMPLE 100

The sheath of any examples herein, particularly example 99, wherein the first fold is configured to move closer to the second fold to shorten the folded portion at a local axial location during passage of the medical device through the lumen and wherein shortening of the folded portion corresponds with a local expansion of the lumen.

### EXAMPLE 101

The sheath of any examples herein, particularly examples 99-100, wherein the inner layer extends through a longitudinally extending opening provided in the outer layer when the outer layer is expanded.

### EXAMPLE 102

The sheath of any examples herein, particularly example 101, wherein the longitudinally extending opening is provided between a longitudinally extending edge of the overlapping portion and a longitudinally extending edge of the underlaying portion.

### EXAMPLE 103

The sheath of any examples herein, particularly examples 99-102, wherein the inner layer comprises at least partially etched PTFE.

### EXAMPLE 104

The sheath of any examples herein, particularly example 103, wherein the inner layer comprises a fully etched PTFE.

### EXAMPLE 105

The sheath of any examples herein, particularly example 104, wherein unetched portions of an outer surface of the inner liner extend longitudinally along a length of the inner layer and/or circumferentially around a length of a circumference of the inner layer.

### EXAMPLE 106

The sheath of any examples herein, particularly examples 103 or 105, wherein unetched portions of the inner layer are provided along portions of the inner layer that contact an outer surface of the outer layer.

### EXAMPLE 107

The sheath of any examples herein, particularly examples 99-106, wherein the sheath further comprises a tie layer disposed between the inner layer and the first outer tubular layer and at least partially adhering the inner layer to the first outer tubular layer.

### EXAMPLE 108

The sheath of any examples herein, particularly example 107, wherein unetched portions of the inner layer are provided along those locations excluding the tie layer, wherein the surface of the inner layer adjacent the underlaying portion of the outer layer when the sheath is not expanded, are unetched.

### EXAMPLE 109

The sheath of any examples herein, particularly examples 99-108, wherein the inner layer has a wall thickness ranging between about 0.002 inches and about 0.006 inches.

### EXAMPLE 110

The sheath of any one of any examples herein, particularly examples 107-109, wherein the tie layer extends at least partially around an outer surface of the inner liner.

### EXAMPLE 111

The sheath of any examples herein, particularly example 110, wherein the tie layer extends around an entirety of the outer surface of the inner liner.

### EXAMPLE 112

The sheath of any one of any examples herein, particularly examples 107-111, wherein the tie layer extends at least partially around an inner surface of the first outer tubular layer.

### EXAMPLE 113

The sheath of any one of any examples herein, particularly example 112, wherein the tie layer extends around an entirety of an inner surface of the first outer tubular layer.

### EXAMPLE 114

The sheath of any one of any examples herein, particularly examples 107-113, wherein the tie layer extends between the first outer tubular layer and the overlapping folded portion of the inner layer.

### EXAMPLE 115

The sheath of any examples herein, particularly example 114, wherein the tie layer extends between an outer surface of the overlapping folded portion of the inner layer and a corresponding surface of the overlapping portion of the first outer tubular layer.

### EXAMPLE 116

The sheath of any examples herein, particularly examples 114-115, wherein the tie layer does not extend between an inner surface of the overlapping folded portion of the inner layer and a corresponding surface of the underlaying portion of the outer surface of the first outer tubular layer.

### EXAMPLE 117

The sheath of any one of any examples herein, particularly examples 107-116, wherein the tie layer adheres at least a portion of the inner layer to a corresponding portion of the first outer tubular layer.

### EXAMPLE 118

The sheath of any one of any examples herein, particularly examples 107-117, wherein the tie layer comprises a material having a Shore A durometer less than about 90 Shore A.

### EXAMPLE 119

The sheath of any examples herein, particularly examples 107-118, wherein the tie layer comprises thermoplastic polyurethane.

### EXAMPLE 120

The sheath of any examples herein, particularly example 119, wherein the tie layer comprises an aliphatic polyether-based thermoplastic polyurethane (TPU).

### EXAMPLE 121

The sheath of any examples herein, particularly example 120, wherein the tie layer comprises of Tecoflex™ 80A.

### EXAMPLE 122

The sheath of any examples herein, particularly example 118, wherein the tie layer comprises an aromatic polyether or polyester-based thermoplastic polyurethane.

### EXAMPLE 123

The sheath of any examples herein, particularly example 124, wherein the tie layer comprises of Pellethane™ 80A.

### EXAMPLE 124

The sheath of any one of any examples herein, particularly examples 107-118, wherein the tie layer comprises a polyolefin or polyamide.

### EXAMPLE 125

The sheath of any examples herein, particularly example 124, wherein the tie layer comprises a polyolefin comprising polyethylene, polypropylene, or ethylene vinyl acetate PE, PP, or EVA modified with maleic anhydride.

### EXAMPLE 126

The sheath of any examples herein, particularly example 125, wherein the tie layer comprises an Orevac™ resin.

### EXAMPLE 127

The sheath of any one of any examples herein, particularly examples 107-126, wherein the tie layer has a wall thickness ranging between about 0.002 inches and about 0.005 inches.

### EXAMPLE 128

The sheath of any examples herein, particularly examples 99-127, wherein the first outer tubular layer exerts a radially inward force on the inner layer.

### EXAMPLE 129

The sheath of any examples herein, particularly examples 99-128, wherein the first outer tubular layer comprises of at least one polymeric material.

### EXAMPLE 130

The sheath of any examples herein, particularly examples 99-129, wherein the first outer tubular layer comprises at least one of HDPE, nylon, and polypropylene.

### EXAMPLE 131

The sheath of any examples herein, particularly examples 99-130, wherein the first outer tubular layer has a wall thickness ranging between about 0.007 inches and about 0.013 inches.

### EXAMPLE 132

The sheath of any examples herein, particularly examples 99-131, wherein the elongated tube is bonded to the first outer tubular layer.

### EXAMPLE 133

The sheath of any examples herein, particularly examples 99-132, wherein the elongated tube is bonded to the first outer tubular layer at a proximal end of the first outer tubular layer.

### EXAMPLE 134

The sheath of any examples herein, particularly examples 99-133, wherein the elongated tube is bonded to the outer layer at a distal end of the first outer tubular layer.

### EXAMPLE 135

The sheath of any examples herein, particularly examples 99-134, wherein the elongated tube is bonded to the first outer tubular layer along a length of the first outer tubular layer between the proximal and distal ends of the first outer tubular layer.

### EXAMPLE 136

The sheath of any examples herein, particularly examples 99-135, wherein a distal end of the elongated tube is bonded to the inner layer.

### EXAMPLE 137

The sheath of any examples herein, particularly example 136, wherein the elongated tube is bonded to a distal end surface of the inner layer.

### EXAMPLE 138

The sheath of any examples herein, particularly examples 99-137, wherein the elongated tube is bonded to at least one of a proximal end of the first outer tubular layer, a distal end of the first outer tubular layer, and a distal end of the inner layer by a chemical and/or mechanical fastener.

### EXAMPLE 139

The sheath of any examples herein, particularly example 138, wherein the mechanical fastener includes thermally-bonded coupling between the elongated tube and at least one of the first outer tubular layer and the inner layer.

### EXAMPLE 140

The sheath of any examples herein, particularly examples 99-139, wherein the elongated tube extends over an entire length of the first outer tubular layer.

### EXAMPLE 141

The sheath of any examples herein, particularly examples 99-140, further comprising a lubricant disposed between the elongated tube and the first outer tubular layer.

### EXAMPLE 142

The sheath of any examples herein, particularly example 141, wherein the lubricant is provided along the portion of the folded portion of the inner layer extending along the outer surface of the first outer tubular layer.

### EXAMPLE 143

The sheath of any examples herein, particularly example 141, wherein the lubricant is applied as a band around a portion of the circumference of the first outer tubular layer, the band of lubricant extending longitudinally along a length of the first outer tubular layer.

### EXAMPLE 144

The sheath of any examples herein, particularly examples 141-143, wherein the lubricant comprises a curable material.

### EXAMPLE 145

The sheath of any examples herein, particularly examples 141-143, wherein the lubricant comprises a medical-grade silicone.

### EXAMPLE 146

The sheath of any examples herein, particularly examples 141-143 wherein the lubricant comprises at least one of Med10-6670, Duraglide™, and/or Christo-Lube™.

### EXAMPLE 147

A sheath for delivering a medical device, the sheath comprising: a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

### EXAMPLE 148

A sheath of any examples herein, particularly examples 1-45, further comprising: a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; a coiled wire along a length of the sheath, the coil wire providing uniform bending of the sheath to prevent kinking: and wherein the elongated tube is positioned such that the inner surface of the elongated tube overlies at least a portion of the outer surface of the first outer tubular layer, and wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpended configuration as the prosthetic device passes through the lumen.

### EXAMPLE 149

The sheath of any examples herein, particularly example 148, further comprising a tie layer provided between the inner layer and the first outer tubular layer and at least partially adhering the inner layer to the first outer tubular layer,

### EXAMPLE 150

The sheath of any examples herein, particularly example 148 or 149, wherein the coiled wire is embedded in the first outer tubular layer.

### EXAMPLE 151

The sheath of any examples herein, particularly examples 148-150, wherein the coiled wire is co-extruded with the first outer tubular layer.

### EXAMPLE 152

The sheath of any examples herein, particularly example 151, wherein the coiled wire is provided between the first outer tubular layer and the tie layer.

### EXAMPLE 153

The sheath of any examples herein, particularly example 152, wherein the coiled wire is embedded at least partially within both the first outer tubular layer and the tie layer.

### EXAMPLE 154

The sheath of any examples herein, particularly example 153, wherein the coiled wire is provided to an outer surface of the tie layer, and the first outer tubular layer is reflowed over.

### EXAMPLE 155

The sheath of any examples herein, particularly examples 148-154, wherein the coiled wire is composed of a metal or a polymer wire.

### EXAMPLE 156

The sheath of any examples herein, particularly examples 148-155, wherein the coiled wire is composed of at least one of PET, PEEK, stainless steel, or nitinol.

### EXAMPLE 157

The sheath of any examples herein, particularly examples 148-156, wherein the coiled wire defines a helical-shaped path around the longitudinal axis of the sheath.

### EXAMPLE 158

The sheath of any examples herein, particularly examples 148-157, wherein the coiled wire defines an overlapping helical-shaped path around the longitudinal axis of the sheath.

### EXAMPLE 159

The sheath of any examples herein, particularly example 158, wherein the overlapping helical-shaped path defines a continuous diamond pattern along a length of the sheath.

### EXAMPLE 160

The sheath of any examples herein, particularly examples 148-159, wherein the coiled wire is a flat wire.

### EXAMPLE 161

The sheath of any examples herein, particularly examples 148-160, wherein the coiled wire is a round wire.

### EXAMPLE 162

The sheath of any examples herein, particularly examples 148-161, wherein the coiled wire has a thickness of about 0.002″ to about 0.008″.

### EXAMPLE 163

The sheath of any examples herein, particularly examples 148-162, wherein a distance between adjacent coils of the coiled wire corresponds to a diameter of the coiled wire.

### EXAMPLE 164

The sheath of any examples herein, particularly examples 148-163, wherein a distance between adjacent coils of the coiled wire is about 0.006″.

### EXAMPLE 165

The sheath of any examples herein, particularly example 164, wherein the coiled wire has a diameter of about 0.006″.

### EXAMPLE 166

The sheath of any one of any examples herein, particularly examples 148-165, wherein a lubricant is provided between the elongated tube and the first outer tubular layer for reducing friction during expansion of the sheath.

### EXAMPLE 167

The sheath of any examples herein, particularly example 166, wherein the lubricant is provided on an outer surface of the outer layer proximate to a longitudinally extending edge of the overlapping portion.

### EXAMPLE 168

The sheath of any examples herein, particularly example 167, wherein at least a portion of the folded portion of the inner layer extends beyond the longitudinally extending edge of the overlapping portion, and along an outer surface of the first outer tubular layer, wherein the lubricant is provided along the portion the folded portion of the inner layer extending along the outer surface of the first outer tubular layer for reducing friction between the inner layer and the elongated tube during expansion of the sheath.

### EXAMPLE 169

A sheath for delivering a medical device, the sheath comprising: a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; a coiled wire along a length of the sheath, the coil wire providing uniform bending of the sheath to prevent kinking; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpended configuration as the prosthetic device passes through the lumen,

### EXAMPLE 170

The sheath of any examples herein, particularly examples 1-159, further comprising a hydrophilic coating disposed on an outer surface of the elongated tube.

### EXAMPLE 171

The sheath of any examples herein, particularly examples 1-170, wherein the sheath comprises a radiopaque material.

### EXAMPLE 172

A method of making a sheath having a proximal end and a distal end and comprising: a) extruding a tubular body to form an elongated tube comprising a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; b) disposing the elongated tube on the sheath such that the elongated tube forms an outer layer of the sheath, and wherein the elongated tube is positioned at at least the proximal end of the sheath and extending along at least a portion of a length of the sheath, wherein the elongated tube is configured to reversibly expand from an initial diameter do in an unexpended position to an expanded diameter dₑ in an expanded position upon passage of a medical device; and wherein the formed sheath exhibits at least a 10% reduction in an insertion force when compared with a substantially identical reference sheath that does not comprise the first polymer layer.

### EXAMPLE 173

The method of any examples herein, particularly example 172, wherein the elongated tube comprises a second polymer layer comprising a second compound composition comprising from 0 wt % to 100 wt % of a second polymer comprising polyether block amide, a polyurethane, or a composition thereof.

### EXAMPLE 174

The method of any examples herein, particularly example 173, wherein a step of extruding comprises co-extruding the first polymer layer and the second polymer layer.

### EXAMPLE 175

The method of any examples herein, particularly example 173 or 174, wherein the second polymer layer is substantially free of an inorganic filler.

### EXAMPLE 176

The method of any examples herein, particularly examples 173-175, wherein the second polymer layer is substantially free of a solid lubricant filler.

### EXAMPLE 177

The method of any examples herein, particularly examples 173-176, wherein the second polymer layer has an average durometer larger than the first polymer layer.

### EXAMPLE 178

The method of any examples herein, particularly examples 173-177, wherein the first polymer layer defines the inner surface of the elongated tube.

### EXAMPLE 179

The method of any examples herein, particularly examples 173-178, wherein the second polymer layer defines the outer surface of the elongated tube.

### EXAMPLE 180

The method of any examples herein, particularly examples 173-179, wherein the first polymer layer defines the outer surface of the elongated tube.

### EXAMPLE 181

The method of any examples herein, particularly examples 173-180, wherein one or more additional polymer layers are disposed between the first polymer layer and the second polymer layer.

### EXAMPLE 182

The method of any examples herein, particularly examples 172-181, further comprising providing a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; disposing the elongated tube such that the inner surface of the elongated tube overlies at least a portion of the outer surface of the inner liner and forms the outer layer of the sheath.

### EXAMPLE 183

The method of any examples herein, particularly examples 172-182, further comprising: providing an inner tubular layer comprising a longitudinal slit and partially defining an inner lumen and a first outer tubular layer enveloping the inner layer, the outer tubular layer comprising a longitudinally extending, folded flap that overlies a portion of an outer surface of the outer layer when the sheath is in an unexpanded state, and disposing the elongated tube such that the inner surface of the elongated tube overlies at least a portion of the outer surface of the first outer tubular layer.

### EXAMPLE 184

The method of any examples herein, particularly examples 172-182, further comprising: providing a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer and a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; disposing the elongated tube such that the inner surface of the elongated tube overlies at least a portion of the outer surface of the discontinuous first outer tubular layer ; wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

### EXAMPLE 185

The method of any examples herein, particularly examples 172-182, further comprising: providing: a) a continuous inner layer defining a lumen therethrough, the inner layer including a first fold and a second fold and an overlapping folded portion extending circumferentially between the first and second folds, the folded portion comprising overlap in a radial direction of at least two thicknesses of the inner layer; b) a discontinuous first outer tubular layer extending at least partially around the inner layer, the first outer tubular layer having an overlapping portion and an underlaying portion, at least a portion of the folded portion of the inner layer is positioned between the overlapping portion and the underlaying portion; and c) a coiled wire along a length of the sheath, the coil wire providing uniform bending of the sheath to prevent kinking; and disposing the elongated tube such that the inner surface of the elongated tube overlies at least a portion of the outer surface of the first outer tubular layer, and wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

### EXAMPLE 186

The method of any examples herein, particularly examples 173-182, further comprising providing: a) an expandable tubular inner liner extending along the length of the sheath and comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion; b) a first outer tubular layer extending at least partially along the length of the sheath and having an inner surface and an outer surface, wherein the inner surface of the first outer tubular layer further extends at least partially around the outer surface of the inner liner such that at least a portion of the inner surface of the first outer tubular layer is positioned adjacent to the outer surface of the at least one folded portion of the inner liner; and disposing the elongated tube such that the inner surface of the elongated tube overlies at least a portion of the outer surface of the first outer tubular layer, and wherein at least a portion of the sheath is configured to locally expand from an unexpanded configuration in which the lumen has a first diameter to an expanded configuration in which the lumen has a second diameter larger than the first diameter due to an outwardly directed radial force exerted by a medical device against the inner layer, and then locally contract at least partially back to the unexpanded configuration as the prosthetic device passes through the lumen.

### EXAMPLE 187

A sheath for delivering a medical device, wherein the sheath has a proximal and a distal end and comprises an elongated tube forming an outer layer of the sheath that is positioned at at least the proximal end of the sheath and extending along at least a portion of a length of the sheath, having an inner surface and an outer surface, and wherein the elongated tube comprises a first polymer layer, wherein the first polymer layer comprises a first compound composition comprising from greater than 0 wt % to less than 100 wt % of a first polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; wherein the elongated tube is configured to reversibly expand from an initial diameter do in an unexpended position to an expanded diameter dₑ in an expanded position upon passage of a medical device; and wherein the sheath exhibits at least a 10% reduction in an insertion force when compared with a substantially identical reference sheath that does not comprise the first polymer layer; and wherein the elongated tube is substantially kink resistant.

### EXAMPLE 188

The sheath of any examples herein, particularly example 187, wherein a durometer of the first polymer at a proximal end of the elongated tube is different from a durometer of the first polymer at a distal end of the elongated tube and has a Shore D from about 20 D to about 35 D.

### EXAMPLE 189

The sheath of any examples herein, particularly examples 187-188, wherein the first polymer comprises polyether block amide elastomer.

### EXAMPLE 190

The sheath of any examples herein, particularly examples 187-188, wherein the first polymer comprises polyurethane.

### EXAMPLE 191

The sheath of any examples herein, particularly examples 187-191, wherein the inorganic filler comprises bismuth oxychloride, barium sulfate, bismuth subcarbonate, calcium carbonate, aluminum trihydrate, barite, kaolin clay, limestone, or any combination thereof and is present in an amount of at least about 10% based on a total weight of the first compound composition.

### EXAMPLE 192

The sheath of any examples herein, particularly examples 187-191, wherein the inorganic filler is present in an amount of less than about 50% based on a total weight of the first compound composition.

### EXAMPLE 193

The sheath of any examples herein, particularly examples 187-192, wherein the solid lubricant comprises a PTFE filler.

### EXAMPLE 194

The sheath of any examples herein, particularly examples 187-193, wherein the first compound composition further comprises at least one tackiness reducing compound present in an amount from about 1% to about 20% based on a total weight of the first compound composition.

### EXAMPLE 195

The sheath of any examples herein, particularly examples 187-194, wherein the elongated tube comprises two or more polymer layers.

### EXAMPLE 196

The sheath any examples herein, particularly example 195, wherein the elongated tube comprises at least a second polymer layer comprising a second compound composition comprising from greater than 0 wt % to 100 wt % of a second polymer comprising polyether block amide, a polyurethane, or a composition thereof; and wherein the second polymer has a Shore A Durometer from about 20 A to about 65 A.

### EXAMPLE 197

The sheath of any examples herein, particularly example 196, wherein the second compound composition further comprises up to 20% of tackiness reducing additive based on a total weight of the second compound composition.

### EXAMPLE 198

The sheath of any examples herein, particularly examples 196-197, wherein the second polymer layer comprises polyurethane.

### EXAMPLE 199

The sheath of any examples herein, particularly examples 196-198, wherein the elongated tube has a predetermined thickness and wherein at least about 50% of the predetermined thickness comprises the first and/or the second compound composition.

### EXAMPLE 200

The sheath of any examples herein, particularly examples 196-199, wherein one or more additional polymer layers are disposed between the first polymer layer and the second polymer layer.

### EXAMPLE 201

The sheath of any examples herein, particularly example 200, wherein the one or more additional polymer layers comprise at least one intermediate reinforcement layer extending axially at at least a portion of a length of the elongated tube.

### EXAMPLE 202

The sheath of any examples herein, particularly example 201, wherein the at least one intermediate reinforcement layer comprises the first polymer, the second polymer, a polyolefin-based polymer, or a combination thereof.

### EXAMPLE 203

The sheath of any examples herein, particularly example 201 or 202, wherein the at least one intermediate reinforcement layer comprises a material having a Shore D durometer from about 45 D to about 76 D.

### EXAMPLE 204

The sheath of any examples herein, particularly examples 201-203, wherein the at least one intermediate reinforcement layer is configured to thermally bond with the first polymer layer, the second polymer layer, or a combination thereof.

### EXAMPLE 205

The sheath of any examples herein, particularly examples 187-204, wherein the elongated tube exhibits a friction force of less than about 7 N in the dry state against a substrate surface comprising one or more of polytetrafluoroethylene or high-density polyethylene.

### EXAMPLE 206

The sheath of any examples herein, particularly examples 187-205, wherein the elongated tube exhibits a hoop direction force at a 10 mm extension of less than about 8 N.

### EXAMPLE 207

The sheath of any examples herein, particularly examples 187-206, the elongated tube exhibits an elongation at break of ranging between about 600% and about 800%.

### EXAMPLE 208

The sheath of any examples herein, particularly examples 187-207, further comprising an expandable tubular inner liner extending along the length of the sheath and comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion; and a first outer tubular layer extending at least partially along the length of the sheath and having an inner surface and an outer surface, wherein the inner surface of the first outer tubular layer further extends at least partially around the outer surface of the inner liner such that at least a portion of the inner surface of the first outer tubular layer is positioned adjacent to the outer surface of the at least one folded portion of the inner liner; wherein the elongated tube is positioned such that at least a portion of the inner surface of the elongated tube overlies at least a portion of the outer surface of the first outer tubular layer; and wherein the sheath exhibits an insertion force of less than about 55 N when a medical device is pushed through the sheath.

### EXAMPLE 209

The sheath of any examples herein, particularly example 208, wherein the expandable tubular inner liner comprises polytetrafluoroethylene; or wherein the first outer tubular layer comprises a high-density polyethylene.

### EXAMPLE 210

A sheath for delivering a medical device, wherein the sheath has a proximal and a distal end and comprises: an expandable tubular inner liner comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion; a first outer tubular layer having an inner surface and an outer surface, wherein the inner surface of the first outer tubular layer extends at least partially around the outer surface of the inner liner such that at least a portion of the inner surface of the first outer tubular layer is positioned adjacent to the outer surface of the at least one folded portion of the inner liner; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition.

### EXAMPLE 211

A sheath for delivering a medical device, wherein the sheath has a proximal and a distal end and comprises: an expandable tubular inner liner comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion and wherein the outer surface of the inner liner is selectively etched; a first outer tubular layer having an inner surface and an outer surface, wherein the inner surface of the outer layer extends at least partially around the outer surface of the inner liner such that at least a portion of the inner surface of the outer layer is positioned adjacent to at least a portion of the outer surface of the at least one folded portion of the inner liner; and an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition.

### EXAMPLE 212

The sheath of any examples herein, particularly examples 187-211, further comprising a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and wherein the elongated tube is positioned such that the inner surface of the elongated tube overlies at least a portion of the outer surface of the inner liner.

### EXAMPLE 213

The sheath of any examples herein, particularly example 212, further comprising a braid positioned between the inner liner and the elongated tube.

### EXAMPLE 214

The sheath of any examples herein, particularly examples 212 or 213, further comprising an additional outer layer positioned in between the inner liner and the elongated tube.

### EXAMPLE 215

The sheath of any examples herein, particularly examples 212-214, wherein the sheet comprises a high-density polyethylene, polypropylene, polyamide, fluoropolymer, copolymers thereof, or blends thereof.

### EXAMPLE 216

The sheath of any examples herein, particularly examples 212-215, wherein an amount of a first lubricant is disposed between at least a portion of the inner liner and at least a portion of the inner surface of the elongated tube.

### EXAMPLE 217

The sheath of any examples herein, particularly examples 212-216, wherein an amount of a second lubricant is disposed between at least a portion of the overlying portion of the sheet and at least a portion of the sliding portions of the sheet.

### EXAMPLE 218

The sheath of any examples herein, particularly examples 213-217, wherein the braid comprises at least one filament comprising stainless steel, nitinol, a polymer material, or a composite material.

### EXAMPLE 219

A sheath for delivering a medical device, wherein the sheath has a proximal and a distal end and comprises: a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of the cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and an elongated tube forming an outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the inner liner, wherein the elongated tube comprises a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition.

### EXAMPLE 220

A method of making a sheath having a proximal end and a distal end and comprising: a) extruding a tubular body to form an elongated tube comprising a first polymer layer, wherein the first polymer layer comprises a first compound composition comprising from greater than 0% to less than 100% of a polymer comprising a polyether block amide, a polyurethane, or a combination thereof based on a total weight of the first compound composition; less than about 65% of an inorganic filler based on a total weight of the first compound composition; and up to about 20% of a solid lubricant filler based on a total weight of the first compound composition; b) disposing the elongated tube on the sheath such that the elongated tube forms an outer layer of the sheath, and wherein the elongated tube is positioned at at least the proximal end of the sheath and extending along at least a portion of a length of the sheath, wherein the elongated tube is configured to reversibly expand from an initial diameter do in an unexpended position to an expanded diameter dₑ in an expanded position upon passage of a medical device; and wherein the formed sheath exhibits at least a 10% reduction in an insertion force when compared with a substantially identical reference sheath that does not comprise the first polymer layer.

### EXAMPLE 221

The method of any examples herein, particularly example 220, wherein the elongated tube comprises a second polymer layer comprising a second compound composition comprising from 0 wt % to 100 wt % of a second polymer comprising polyether block amide, a polyurethane, or a composition thereof.

### EXAMPLE 222

The method of any examples herein, particularly example 221, wherein a step of extruding comprises co-extruding the first polymer layer and the second polymer layer.

### EXAMPLE 223

The method of any examples herein, particularly examples 220-222, wherein one or more additional polymer layers are disposed between the first polymer layer and the second polymer layer.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

We claim:

1. A sheath for delivering a medical device, wherein the sheath has a proximal and a distal end and comprises
an elongated tube forming an outer layer of the sheath that is positioned at at least the proximal end of the sheath and extending along at least a portion of a length of the sheath, having an inner surface and an outer surface, and wherein the elongated tube comprises a first polymer layer, wherein the first polymer layer comprises a first compound composition comprising
from greater than 0 wt % to less than 100 wt % of a first polymer comprising a polyether block amide, a polyurethane, or a combination thereof based on a total weight of the first compound composition;
less than about 65% of an inorganic filler based on a total weight of the first compound composition; and
up to about 20% of a solid lubricant filler based on a total weight of the first compound composition;
wherein the elongated tube is configured to reversibly expand from an initial diameter do in an unexpended position to an expanded diameter de in an expanded position upon passage of a medical device; and
wherein the sheath exhibits at least a 10% reduction in an insertion force when compared with a substantially identical reference sheath that does not comprise the first polymer layer; and wherein the elongated tube is substantially kink resistant.

2. The sheath of claim 1, wherein a durometer of the first polymer at a proximal end of the elongated tube is different from a durometer of the first polymer at a distal end of the elongated tube and has a Shore D from about 20 D to about 35 D.

3. The sheath of claim 1, wherein the first polymer comprises polyether block amide elastomer, polyurethane, or a combination thereof.

4. The sheath of claim 1, wherein the inorganic filler comprises bismuth oxychloride, barium sulfate, bismuth subcarbonate, calcium carbonate, aluminum trihydrate, barite, kaolin clay, limestone, or any combination thereof and is present in an amount of at least about 10% to less than about 50% based on a total weight of the first compound composition.

5. The sheath of claim 1, wherein the solid lubricant filler comprises a PTFE filler.

6. The sheath of claim 1, wherein the first compound composition further comprises at least one tackiness reducing compound present in an amount from about 1% to about 20% based on a total weight of the first compound composition.

7. The sheath of claim 1, wherein the elongated tube comprises two or more polymer layers, and wherein at least a second polymer layer comprising a second compound composition comprising from greater than 0 wt % to 100 wt % of a second polymer comprising polyether block amide, a polyurethane, or a composition thereof; and wherein the second polymer has a Shore A Durometer from about 20 A to about 65 A.

8. The sheath of claim 7, wherein the second compound composition further comprises up to 20% of tackiness reducing additive based on a total weight of the second compound composition.

9. The sheath of claim 7, wherein the second polymer comprises polyurethane.

10. The sheath of claim 7, wherein the elongated tube has a predetermined thickness and wherein at least about 50% of the predetermined thickness comprises the first and/or the second compound composition.

11. The sheath of claim 7, wherein one or more additional polymer layers are disposed between the first polymer layer and the second polymer layer.

12. The sheath of claim 11, wherein the one or more additional polymer layers comprise at least one intermediate reinforcement layer extending axially at at least a portion of a length of the elongated tube, and wherein the at least one intermediate reinforcement layer comprises the first polymer, the second polymer, a polyolefin-based polymer, or a combination thereof.

13. The sheath of claim 12, wherein the at least one intermediate reinforcement layer comprises a material having a Shore D durometer from about 45 D to about 76 D.

14. The sheath of claim 1, wherein the elongated tube exhibits a friction force of less than about 10 N in the dry state against a substrate surface comprising one or more of polytetrafluoroethylene or high-density polyethylene, and/or wherein the elongated tube exhibits a hoop direction force at 10 mm extension of less than about 8 N; and/or the elongated tube exhibits an elongation at break of ranging between about 600% and about 800%.

15. A sheath for delivering a medical device, wherein the sheath has a proximal and a distal end and comprises:
an expandable tubular inner liner comprising at least one folded portion, wherein the expandable inner liner has an inner surface, and an outer surface, wherein the inner surface of the expandable inner liner defines a lumen and forms an inner surface of the at least one folded portion, and wherein the outer surface extends circumferentially to form an outer surface of the at least one folded portion;
a first outer tubular layer having an inner surface and an outer surface, wherein the inner surface of the first outer tubular layer extends at least partially around the outer surface of the inner liner such that at least a portion of the inner surface of the first outer tubular layer is positioned adjacent to the outer surface of the at least one folded portion of the inner liner; and
an elongated tube forming a second outer layer having an inner surface and an outer surface and wherein the elongated tube is positioned at at least the proximal end of the sheath and extending along at least a portion of a length of the sheath, such that the inner surface of the elongated tube overlies at least a portion of the outer surface of the first outer tubular layer, wherein
the elongated tube comprises a first polymer layer, wherein the first polymer layer comprises a first compound composition comprising
from greater than 0% to less than 100% of a polymer comprising a polyether block amide, a polyurethane, or a combination thereof based on a total weight of the first compound composition;
less than about 65% of an inorganic filler based on a total weight of the first compound composition; and
up to about 20% of a solid lubricant filler based on a total weight of the first compound composition.

16. The sheath of claim 1, further comprising
a variable diameter inner liner comprising a sheet having a first edge and a second edge and is defined by an inner surface and an outer surface, wherein the sheet is wound in a spiral configuration such that at least a portion of the inner surface of the sheet overlays at least a portion of the outer surface of the sheet and wherein the first edge of the sheet is slidable along at least a portion the inner surface of the sheet and the second edge is slidable along at least a portion of the outer surface of the sheet, wherein the inner surface of the sheet defines a lumen of a cylinder having a longitudinal axis; wherein the variable diameter inner liner is configured to reversible expand from a predetermined rest diameter dᵣ to an expanded diameter d₁ by sliding the first edge of the sheet along at least a portion of the inner surface and sliding the second edge of the sheet along the at least a portion of outer surface, during application of a radial outward force by passage of a medical device through the lumen of the inner liner; and wherein the elongated tube is positioned such that the inner surface of the elongated tube overlies at least a portion of the outer surface of the inner liner.

17. The sheath of claim 16, further comprising a braid positioned between the inner liner and the elongated tube.

18. A method of making a sheath having a proximal end and a distal end and comprising:
a) extruding a tubular body to form an elongated tube comprising a first polymer layer, wherein the first polymer layer comprises a first compound composition comprising
from greater than 0% to less than 100% of a polymer comprising a polyether block amide, a polyurethane, or a combination thereof based on a total weight of the first compound composition;
less than about 65% of an inorganic filler based on a total weight of the first compound composition; and
up to about 20% of a solid lubricant filler based on a total weight of the first compound composition;
b) disposing the elongated tube on the sheath such that the elongated tube forms an outer layer of the sheath, and wherein the elongated tube is positioned at at least the proximal end of the sheath and extending along at least a portion of a length of the sheath, wherein the elongated tube is configured to reversibly expand from an initial diameter do in an unexpended position to an expanded diameter de in an expanded position upon passage of a medical device; and
wherein the formed sheath exhibits at least a 10% reduction in an insertion force when compared with a substantially identical reference sheath that does not comprise the first polymer layer.

19. The method of claim 18, wherein the elongated tube comprises a second polymer layer comprising a second compound composition comprising from 0 wt % to 100 wt % of a second polymer comprising polyether block amide, a polyurethane, or a composition thereof.

20. The method of claim 19 wherein a step of extruding comprises co-extruding the first polymer layer and the second polymer layer.
